(19) United States
(12) Patent Application Publication
(10) Pub. No.: US 2021/0295948 A1
(43) Pub. Date: Sep. 23, 2021

(54) SYSTEMS AND METHODS FOR ESTIMATING CELL SOURCE FRACTIONS USING METHYLATION INFORMATION

(71) Applicant: GRAIL, Inc., Menlo Park, CA (US)

(72) Inventors: Jing XIANG, San Carlos, CA (US); Robert Abe Paine Calef, Redwood City, CA (US)

(21) Appl. No.: 17/127,813

(22) Filed: Dec. 18, 2020

Related U.S. Application Data
(63) Continuation of application No. 62/950,071, filed on Dec. 18, 2019.

(51) Int. Cl. G16B 20/20; G16B 40/00

(52) U.S. Cl. CPC G16B 20/20; G16B 40/00

(57) ABSTRACT

A method of identifying a plurality of features for estimating subject cell source fraction is provided. For each respective training subject in a plurality of training subjects, a corresponding methylation pattern of each respective cell-free fragment in a corresponding training plurality of cell-free fragments and a corresponding subject cancer indication is obtained. Each cell-free fragment is mapped to a bin in a plurality of bins, each bin representing a portion of a human reference genome. A cell-free fragment cancer condition is assigned to each cell-free fragment, as a function of a classifier upon inputting a corresponding methylation pattern of the respective cell-free fragment into the classifier. A measure of association is determined for each bin between the subject cancer condition and the cell-free fragment cancer condition. The plurality of features for estimating subject cell source fraction are identified as a subset of the plurality of bins.

### CROSS-REFERENCE TO RELATED APPLICATIONS

This application claims priority to U.S. Provisional Patent Application No. 62/950,071, entitled “Systems and Methods for Estimating Cell Source Fractions using Methylation Information,” filed Dec. 18, 2019, the contents of which are hereby incorporated by reference in its entirety for all purposes.

### TECHNICAL FIELD

This specification describes using nucleic acids, in particular cell-free nucleic acid samples, of a subject to estimate cell source fractions, for example tumor fraction, in biological samples obtained from a subject.

### BACKGROUND

The increasing knowledge of the molecular basis for cancer and the rapid development of next generation sequencing techniques are advancing the study of early molecular alterations involved in cancer development in body fluids. Large scale sequencing technologies, such as next generation sequencing (NGS), have afforded the opportunity to achieve sequencing at costs that are less than one U.S. dollar per million bases, and in fact costs of less than ten U.S. cents per million bases have been realized. Specific genetic and epigenetic alterations associated with such cancer development are found in plasma, serum, and urine cell-free DNA (cfDNA). Such alterations could potentially be used as diagnostic biomarkers for several classes of cancers (see Salvi et al., 2016, Onco Targets Ther. 9:6549-6559).

Cell-free DNA (cfDNA) can be found in serum, plasma, urine, and other body fluids (Chan et al., 2003, Ann Clin Biochem. 40(Pt 2):122-130) representing a “liquid biopsy,” which is a circulating picture of a specific disease (see De Mattos-Arruda and Caldas, 2016, Mol Oncol. 10(3):464-474). This represents a potential, non-invasive method of screening for a variety of cancers.

The existence of cfDNA was demonstrated by Mandel and Metais decades ago (Mandel and Metais, 1948, C R Seances Soc Biol Fil. 142(3-4):241-243). cfDNA originates from necrotic or apoptotic cells, and it is generally released by all types of cells. Stroun et al. further showed that specific cancer alterations could be found in the cfDNA of patients (see, Stroun et al., 1989 Oncology 1989 46(5):318-322). A number of subsequent articles confirmed that cfDNA contains specific tumor-related alterations, such as mutations, methylation, and copy number variations (CNVs), thus confirming the existence of circulating tumor DNA (ctDNA) (see, Goessl et al., 2000 Cancer Res. 60(21):5941-5945 and Frenel et al., 2015, Clin Cancer Res. 21(20):4586-4596).

cfDNA in plasma or serum is well characterized, while urine cfDNA (ucfDNA) has been traditionally less characterized. However, recent studies demonstrated that ucfDNA could also be a promising source of biomarkers (e.g., Casadio et al., 2013, Urol Oncol. 31(8):1744-1750).

In blood, apoptosis is a frequent event that determines the amount of cfDNA. In cancer patients, however, the amount of cfDNA seems to be also influenced by necrosis (see Hao et al., 2014, Br J Cancer 111(8):1482-1489 and Zonta et al., 2015 Adv Clin Chem. 70:197-246). Since apoptosis seems to be the main release mechanism circulating cfDNA has a size distribution that reveals an enrichment in short fragments of about 167 base pairs, (see, Heitzer et al., 2015, Clin Chem. 61(1):112-123 and Lo et al., 2010, Sci Transl Med. 2(61):61ra91) corresponding to nucleosomes generated by apoptotic cells.

The amount of circulating cfDNA in serum and plasma seems to be significantly higher in patients with tumors than in healthy controls, especially in those with advanced-stage tumors than in early-stage tumors (see, Sozzi et al., 2003, J Clin Oncol. 21(21):3902-3908, Kim et al., 2014, Ann Surg Treat Res. 86(3):136-142; and Shao et al., 2015, Oncol Lett. 10(6):3478-3482). The variability of the amount of circulating cfDNA is higher in cancer patients than in healthy individuals, (see, Heitzer et al., 2013, Int J Cancer. 133(2):346-356) and the amount of circulating cfDNA is influenced by several physiological and pathological conditions, including proinflammatory diseases (see, Raptis and Menard, 1980, J Clin Invest. 66(6):1391-1399, and Shapiro et al., 1983, Cancer 51(11):2116-2120).

Methylation status and other epigenetic modifications are known to be correlated with the presence of some disease conditions such as cancer (see Jones, 2002, Oncogene 21:5358-5360). Additionally, specific patterns of methylation have been determined to be associated with particular cancer conditions (see Paska and Hudler, 2015, Biochemia Medica 25(2):161-176). Warton and Samimi have demonstrated that methylation patterns can be observed even in cell-free DNA (Warton and Samimi, 2015, Front Mol Biosci, 2(13) doi: 10.3389/fmolb.2015.00013).

Given the promise of circulating cfDNA, as well as other forms of genotypic data, as a diagnostic indicator, methods for assessing such data to identify epigenetic patterns are needed in the art.

### SUMMARY

The present disclosure addresses the shortcomings identified in the background by providing robust techniques for determining cell source fractions, such as tumor fraction, in biological samples obtained from a subject using cfDNA. The combination of methylation data with whole genome, or targeted genome, sequencing data provides additional diagnostic power beyond previous screening methods.

Technical solutions (e.g., computing systems, methods, and non-transitory computer readable storage mediums) for addressing the above identified problems with analyzing datasets are provided in the present disclosure.

The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

A. Embodiments that Estimate Cell Source Fraction Based at Least in Part on a Subset of Bins that are Identified by Ratios of Cancer-Derived Fragments in Each Bin.

One aspect of the present disclosure provides a method of identifying a plurality of features for estimating subject cell source fraction. The method comprises, at a computer system having one or more processors, and memory storing one or more programs for execution by the one or more processors, obtaining a training dataset, in electronic form. The training dataset comprises, for each respective training subject in a plurality of training subjects: a) a corresponding methylation pattern of each respective cell-free fragment in a corresponding training plurality of cell-free fragments, and b) a subject cancer indication of the respective training subject. The corresponding methylation pattern of each respective cell-free fragment (i) is determined by a methylation sequencing of one or more nucleic acid samples comprising the respective fragment in a corresponding biological sample obtained from the respective training subject, and (ii) comprises a methylation state of each CpG site in a corresponding plurality of CpG sites in the respective fragment. The subject cancer condition is one of a first cancer condition and a second cancer condition. The method further comprises mapping each cell-free fragment in each plurality of cell-free fragments to a bin in a plurality of bins. Here, each respective bin in the plurality of bins represents a corresponding portion of a human reference genome, thereby obtaining a plurality of training sets of cell-free fragments, and each training set of cell-free fragments is mapped to a different bin in the plurality of bins. The method further comprises assigning a cell-free fragment cancer condition to each respective cell-free fragment in each training set of cell-free fragments in the plurality of training sets of cell-free fragments as a function of an output of a classifier upon inputting a methylation pattern of the respective cell-free fragment into the classifier. The cell-free fragment cancer condition is one of the first cancer condition and the second cancer condition. The method further comprises determining, for each respective bin in the plurality of bins, a corresponding measure of association between (a) the subject cancer condition of respective training subjects in the plurality of training subjects and (b) the cell-free fragment cancer condition of respective cell-free fragments in the corresponding training set of cell-free fragments mapping to the respective bin. In some embodiments this method of association is a correlation calculation. In some embodiments this method of association is a mutual information calculation. In some embodiments this method of association is by way of calculating a distance metric (e.g., a Manhattan distance, a maximum value, a normalized Euclidean distance, a normalized Manhattan distance, a dice coefficient, a cosine distance or a Jaccard coefficience, etc.). The method continues by identifying the plurality of features for estimating subject cell source fraction as a subset of the plurality of bins. Each respective bin in the subset of the plurality of bins satisfies a selection criterion based on the corresponding measure of association for the respective bin. For instance, in some embodiments, those bins that have a top ranking measure of association relative to all other bins are deemed to satisfy the selection criterion.

In some embodiments, method further comprises estimating a cell source fraction for a test subject by a procedure that comprises obtaining, in electronic form, a corresponding methylation pattern of each respective cell-free fragment in a test plurality of cell-free fragments. The corresponding methylation pattern of each respective cell-free fragment (i) is determined by a methylation sequencing of one or more nucleic acid samples comprising the respective fragment in a biological sample obtained from the test subject and (ii) comprises a methylation state of each CpG site in a corresponding plurality of CpG sites in the respective fragment. Each cell-free fragment in the test plurality of cell-free fragments is mapped to a bin in the plurality of bins thereby obtaining a plurality of test sets of cell-free fragments, each test set of cell-free fragments mapped to a different bin in the plurality of bins. A cell-free fragment cancer condition is assigned for each respective cell-free fragment in each test set of cell-free fragments the plurality of test sets of cell-free fragments as the function of a an output of the classifier upon inputting a methylation pattern of the respective cell-free fragment into the classifier. A first measure of central tendency of the number of cell-free fragments is computed from the test subject that have been assigned the first cancer condition in each test set of cell-free fragments across the subset of the plurality of bins. A second measure of central tendency of the number of cell-free fragments is computed from the test subject in each test set of cell-free fragments across the subset of the plurality of bins. The cell source fraction for the test subject is then estimated using the first and second measure of central tendency.

In some embodiments, the second cancer condition is absence of cancer, and the cell source fraction for the test subject comprises a cell source fraction for the test subject.

In some embodiments, the classifier has the form:

$$R ( fragment ) ≡ log ( \frac{ℙ ( fragment \vert first cancer condition )}{ℙ ( fragment \vert second cancer condition )} ) .$$

In some such embodiments, [symbol] (fragment|first cancer condition class) is a first model for the first cancer condition, “fragment” refers to the methylation pattern of the respective cell-free fragment, [symbol] (fragment|second cancer condition class) is a second model for the second cancer condition. In such embodiments, the cell-free fragment cancer condition of the respective fragment is assigned the first cancer condition when R(fragment) satisfies a threshold value. In some embodiments, the threshold value is between 1 and 10. In some embodiments, the threshold value is 1, 2, 3, 4, 5, 6, 7, 8, 9, or 10.

In some embodiments, the measure of association I is calculated as:

$$I = {Σ}_{i , j} p ( {x}_{i} , {y}_{j} ) log \frac{p ( {x}_{i} , {y}_{j} )}{p ( {x}_{i} ) p ( {y}_{j} )} .$$

In some such embodiments, i and j are independent indices to the set, xᵢ is the number of training subjects in the plurality of training subjects that have the cancer condition i, yⱼ is a number of training subjects in the plurality of training subjects that have one or more cell-free fragments mapping to the respective bin that are assigned the cancer condition j, p(xᵢ,yⱼ) is

$$\frac{N ( {x}_{i} , {y}_{j} )}{{N}_{T}} ,$$

N (xᵢ,yⱼ) is a number of training subjects in the plurality of training subjects that have the cancer condition i and also have one or more cell-free fragments mapping to the respective bin that are assigned the cancer condition j, N_{T} is the number of training subjects in the plurality of training subjects, p(xᵢ) is xᵢ/N_{T}, and p(yⱼ) is yⱼ/N_{T}.

In some embodiments, the measure of association is a correlation. In some embodiments, the correlation is a Pearson correlation coefficient. In some embodiments, the correlation is performed using an adjusted correlation coefficient, weighted correlation coefficient, reflective correlation coefficient, or scaled correlation coefficient.

In some embodiments, the plurality of bins consists of between 1000 bins and 100,000 bins. In some embodiments, the plurality of bins consists of between 15,000 bins and 80,000 bins. In some embodiments, each respective bin in the plurality of bins has, on average, between 10 and 1200 residues. In some embodiments, each respective bin in the plurality of bins has, on average, between 10 and 10000 residues.

In some embodiments, the first measure of central tendency is an arithmetic mean, a weighted mean, a midrange, a midhinge, a trimean, a Winsorized mean, a mean, or a mode of the number of cell-free fragments from the plurality of test subjects that have been assigned the first cancer condition in each test set of cell-free fragments across the subset of the plurality of bins.

In some embodiments, the second measure of central tendency is an arithmetic mean, a weighted mean, a midrange, a midhinge, a trimean, a Winsorized mean, a mean, or a mode of the number of cell-free fragments from the plurality of test subjects in each test set of cell-free fragments across the subset of the plurality of bins.

In some embodiments, the estimating the cell source fraction comprises dividing the first measure of central tendency by the second measure of central tendency.

In some embodiments, the plurality of training subjects consists of between 10 training subjects and 1000 training subjects.

In some embodiments, the selection criterion specifies selection of the bins having one of the top N measures of association, wherein N is a positive integer of 50 or greater. In some embodiments, N is between 500 and 5000. In some embodiments, N is between 800 and 1500.

In some embodiments, the methylation sequencing is paired-end sequencing. In some embodiments, the methylation sequencing is single-read sequencing. In some embodiments, the corresponding training plurality of cell-free fragments have an average length of less than 500 nucleotides.

In some embodiments, the first cancer condition is cancer and the second cancer condition is absence of cancer.

In some embodiments, the first cancer condition is one of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, or leukemia, and the second cancer condition is absence of cancer.

In some embodiments, the first cancer condition is one of a stage of adrenal cancer, a stage of biliary tract cancer, a stage of bladder cancer, a stage of bone/bone marrow cancer, a stage of brain cancer, a stage of breast cancer, a stage of cervical cancer, a stage of colorectal cancer, a stage of cancer of the esophagus, a stage of gastric cancer, a stage of head/neck cancer, a stage of hepatobiliary cancer, a stage of kidney cancer, a stage of liver cancer, a stage of lung cancer, a stage of ovarian cancer, a stage of pancreatic cancer, a stage of pelvis cancer, a stage of pleura cancer, a stage of prostate cancer, a stage of renal cancer, a stage of skin cancer, a stage of stomach cancer, a stage of testis cancer, a stage of thymus cancer, a stage of thyroid cancer, a stage of uterine cancer, a stage of lymphoma, a stage of melanoma, a stage of multiple myeloma, or a stage of leukemia, and the second cancer condition is absence of cancer.

In some embodiments, the methylation sequencing is whole genome methylation sequencing. In some embodiments, the methylation sequencing is targeted sequencing using a plurality of nucleic acid probes and each bin in the plurality of bins is associated with at least one nucleic acid probe in the plurality of nucleic acid probes.

In some embodiments, the plurality of nucleic acid probes comprises 1,000 or more nucleic acid probes, 2,000 or more nucleic acid probes, 3,000 or more nucleic acid probes, 5,000 or more nucleic acid probes, 10,000 or more nucleic acid probes or between 1,000 nucleic acid and 30,000 nucleic acid probes.

In some embodiments, each bin in the plurality of bins comprises 2, 3, 4, 5, 6, 7, 8, 9, 10, 11, 12, 13, 14, 15, 16, 17, 18, 19, 20 or more CpG sites. In some embodiments, each bin in the plurality of bins comprises 2, 3, 4, 5, 6, 7, 8, 9, 10, 11, 12, 13, 14, 15, 16, 17, 18, 19, 20 or more contiguous CpG sites. In some embodiments, each bin in the plurality of bins consists of between 2 and 100 contiguous CpG sites in a human reference genome.

In some embodiments, the corresponding biological sample is a liquid biological sample. In some embodiments, the corresponding biological sample is a blood sample. In some embodiments, the corresponding biological sample comprises blood, whole blood, plasma, serum, urine, cerebrospinal fluid, fecal, saliva, sweat, tears, pleural fluid, pericardial fluid, or peritoneal fluid of the training subject. In some embodiments, the corresponding biological sample consists of blood, whole blood, plasma, serum, urine, cerebrospinal fluid, fecal, saliva, sweat, tears, pleural fluid, pericardial fluid, or peritoneal fluid of the training subject.

In some embodiments, the methylation state of a respective CpG site in the corresponding plurality of CpG sites in the respective fragment is methylated when the respective CpG site is determined by the methylation sequencing to be methylated, unmethylated when the respective CpG site is determined by the methylation sequencing to not be methylated, and flagged as “other” when the methylation sequencing is unable to call the methylation state of the respective CpG site as methylation or unmethylated.

In some embodiments, the methylation sequencing detects one or more 5-methylcytosine (5mC) and/or 5-hydroxymethylcytosine (5hmC) in the respective fragment.

In some embodiments, the methylation sequencing comprises conversion of one or more unmethylated cytosines or one or more methylated cytosines, in sequence reads of the respective fragment, to a corresponding one or more uracils. In some embodiments, the one or more uracils are detected during the methylation sequencing as one or more corresponding thymines. In some embodiments, the conversion of one or more unmethylated cytosines or one or more methylated cytosines comprises a chemical conversion, an enzymatic conversion, or combinations thereof.

In some embodiments, the first model is a first mixture model comprising a first plurality of sub-models, the second model is a second mixture model comprising a second plurality of sub-models, and each sub-model in the first and second plurality of sub-models represents an independent corresponding methylation model for a source of cell-free fragments in the corresponding biological sample.

In some embodiments, each independent corresponding methylation model is one of a binomial model, beta-binomial model, independent sites model or Markov model.

In some embodiments, two or more sub-models in the first plurality of sub-models are independent sites models, and two or more sub-models in the second plurality of sub-models are independent sites models.

In some embodiments, the method further comprises applying one or more filter conditions to the plurality of cell-free fragments.

In some embodiments, a filter condition in the one or more filter conditions is application of a p-value threshold to the corresponding methylation pattern for each respective cell-free fragment in the plurality of cell-free fragments, where the p-value threshold is representative of how frequently a methylation pattern is observed in a cohort of non-cancer subjects.

In some embodiments, the p-value threshold is between 0.001 and 0.20.

In some embodiments, the cohort comprises at least twenty subjects and the plurality of cell-free fragments comprises at least 10,000 different corresponding methylation patterns.

In some embodiments, the p-value threshold is satisfied for a methylation pattern from the subject when the corresponding methylation pattern for each respective cell-free fragment in the plurality of cell-free fragments has a p-value of 0.10 or less, 0.05 or less, or 0.01 or less.

In some embodiments, a filter condition in the one or more filter conditions is application of a requirement that each respective cell-free fragment in the plurality of cell-free fragments is represented by a threshold number of sequence reads in a corresponding plurality of sequence reads measured from the one or more nucleic acid samples comprising the respective fragment in the corresponding biological sample.

In some embodiments, the threshold number is 2, 3, 4, 5, 6, 7, 8, 9, 10, or an integer between 10 and 100.

In some embodiments, a filter condition in the one or more filter conditions is application of a requirement that each respective cell-free fragment in the plurality of cell-free fragments is represented by a threshold number of cell-free nucleic acids in the one or more nucleic acid samples comprising the respective fragment in the corresponding biological sample.

In some embodiments, the threshold number is 2, 3, 4, 5, 6, 7, 8, 9, 10, or an integer between 10 and 100.

In some embodiments, a filter condition in the one or more filter conditions is application of a requirement that each respective cell-free fragment in the plurality of cell-free fragments have a threshold number of CpG sites.

In some embodiments, the threshold number of CpG sites is at least 1, 2, 3, 4, 5, 6, 7, 8, 9 or 10 CpG sites.

In some embodiments, a filter condition in the one or more filter conditions is a requirement that each respective cell-free fragment in the plurality of cell-free fragments have a length of less than a threshold number of base pairs.

In some embodiments, the threshold number of base pairs is one thousand, two thousand, three thousand, or four thousand contiguous base pairs in length.

In some embodiments, the method further comprises repeating the obtaining, mapping, assigning, computing the first and second measure of central tendency, and estimating the cell source fraction for the test subject at each respective time point in a plurality of time points across an epoch, thus obtaining a corresponding cell source fraction, in a plurality of cell source fractions, for the test subject at each respective time point, and using the plurality of cell source fractions to determine a state or progression of a disease condition in the test subject during the epoch in the form of an increase or decrease of a first cell source fraction over the epoch.

In some embodiments, the epoch is a period of months and each time point in the plurality of time points is a different time point in the period of months.

In some embodiments, the period of months is less than four months.

In some embodiments, the epoch is a period of years and each time point in the plurality of time points is a different time point in the period of years.

In some embodiments, the period of years is between two and ten years.

In some embodiments, the epoch is a period of hours and each time point in the plurality of time points is a different time point in the period of hours.

In some embodiments, the period of hours is between one hour and six hours.

In some embodiments, the method further comprises changing a diagnosis of the test subject when the first cell source fraction of the subject is observed to change by a threshold amount across the epoch.

In some embodiments, the method further comprises changing a prognosis of the test subject when the first cell source fraction of the subject is observed to change by a threshold amount across the epoch.

In some embodiments, the method further comprises changing a treatment of the test subject when the first cell source fraction of the subject is observed to change by a threshold amount across the epoch.

In some embodiments, the threshold is greater than ten percent, greater than twenty percent, greater than thirty percent, greater than forty percent, greater than fifty percent, greater than two-fold, greater than three-fold, or greater than five-fold.

In some embodiments, the tumor fraction for the test subject is between 0.003 and 1.0.

In some embodiments, the method further comprises applying a treatment regimen to the test subject based at least in part, on a value of the cell source fraction for the test subject.

In some embodiments, the treatment regimen comprises applying an agent for cancer to the test subject.

In some embodiments, the agent for cancer is a hormone, an immune therapy, radiography, or a cancer drug.

In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the method further comprises using the cell source fraction for the test subject to evaluate a response of the subject to the agent for cancer.

In some embodiments, the agent for cancer is a hormone, an immune therapy, radiography, or a cancer drug.

In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the method further comprises using the cell source fraction for the test subject to determine whether to intensify or discontinue the agent for cancer in the test subject.

In some embodiments, the test subject has been subjected to a surgical intervention to address the cancer and the method further comprises using the cell source fraction for the test subject to evaluate a condition of the test subject in response to the surgical intervention.

In some embodiments, a bin in the plurality of bins corresponds to a genomic region listed in one or more of Tables 1-24 of International Patent Application No. PCT/US2019/025358 (published as WO2019/195268A2), lists 1-8 of International Patent Application No. PCT/US2019/053509 (published as WO2020/069350A1), and/or lists 1-16 of International Patent Application No. PCT/US2020/015082 (published as WO2020/154682A2), each of which is hereby incorporated herein by reference in its entirety.

In some embodiments, a bin in the plurality of bins maps to at least 30% of a genomic region listed in one or more of Tables 1-24 of International Patent Application No. PCT/US2019/025358 (published as WO2019/195268A2), lists 1-8 of International Patent Application No. PCT/US2019/053509 (published as WO2020/069350A1), and/or lists 1-16 of International Patent Application No. PCT/US2020/015082 (published as WO2020/154682A2).

In some embodiments, a bin in the plurality of bins maps to at least between 50 and 95% of a genomic region listed in one or more of Tables 1-24 of International Patent Application No. PCT/US2019/025358 (published as WO2019/195268A2), lists 1-8 of International Patent Application No. PCT/US2019/053509 (published as WO2020/069350A1), and/or lists 1-16 of International Patent Application No. PCT/US2020/015082 (published as WO2020/154682A2).

In some embodiments, a bin in the plurality of bins maps to between one and 10 unique corresponding genomic region in one or more of Tables 1-24 of International Patent Application No. PCT/US2019/025358 (published as WO2019/195268A2), lists 1-8 of International Patent Application No. PCT/US2019/053509 (published as WO2020/069350A1), and/or lists 1-16 of International Patent Application No. PCT/US2020/015082 (published as WO2020/154682A2).

In some embodiments, each bin in the plurality of bins maps to a single unique corresponding genomic region in one or more of Tables 1-24 of International Patent Application No. PCT/US2019/025358 (published as WO2019/195268A2), lists 1-8 of International Patent Application No. PCT/US2019/053509 (published as WO2020/069350A1), and lists 1-16 of International Patent Application No. PCT/US2020/015082 (published as WO2020/154682A2).

In some embodiments, the training plurality of cell-free fragments, for a respective training subject in the plurality of training subjects, comprises at least 100,000 cell-free fragments.

In some embodiments, the training plurality of cell-free fragments, for each respective training subject in the plurality of training subjects, comprises at least 100,000 cell-free fragments.

In some embodiments, the training plurality of cell-free fragments, for a respective training subject in the plurality of training subjects, comprises at least 1 million cell-free fragments.

In some embodiments, each bin in the plurality of bins consists of less than 100 nucleic acid residues, less than 500 nucleic acid residues, less than 1000 nucleic acid residues, less than 2500 nucleic acid residues, less than 5000 nucleic acid residues, less than 10,000 nucleic acid residues, less than 25,000 nucleic acid residues, less than 50,000 nucleic acid residues, less than 100,000 nucleic acid residues, less than 250,000 nucleic acid residues, or less than 500,000 nucleic acid residues.

Another aspect of the present disclosure provides a computing system for estimating cell source fraction of a subject. The computing system comprises one or more processors and memory storing one or more programs to be executed by the one or more processor. The one or more programs comprises instructions for obtaining a training dataset, in electronic form. The training dataset comprises, for each respective training subject in a plurality of training subjects: a) a corresponding methylation pattern of each respective cell-free fragment in a corresponding training plurality of cell-free fragments, and b) a subject cancer indication of the respective training subject. The corresponding methylation pattern of each respective cell-free fragment (i) is determined by a methylation sequencing of one or more nucleic acid samples comprising the respective fragment in a corresponding biological sample obtained from the respective training subject, and (ii) comprises a methylation state of each CpG site in a corresponding plurality of CpG sites in the respective fragment. The subject cancer condition is one of a first cancer condition and a second cancer condition. The one or more programs further comprise instructions for mapping each cell-free fragment in each plurality of cell-free fragments to a bin in a plurality of bins. Here, each respective bin in the plurality of bins represents a corresponding portion of a human reference genome, thereby obtaining a plurality of training sets of cell-free fragments, and each training set of cell-free fragments is mapped to a different bin in the plurality of bins. The one or more programs further comprise instructions for assigning a cell-free fragment cancer condition to each respective cell-free fragment in each training set of cell-free fragments in the plurality of training sets of cell-free fragments as a function of an output of a classifier upon inputting a methylation pattern of the respective cell-free fragment into the classifier. The cell-free fragment cancer condition is one of the first cancer condition and the second cancer condition. The one or more programs further comprise instructions for determining, for each respective bin in the plurality of bins, a corresponding measure of association I between (a) the subject cancer condition of respective training subjects in the plurality of training subjects and (b) the cell-free fragment cancer condition of respective cell-free fragments in the corresponding training set of cell-free fragments mapping to the respective bin. The one or more programs further comprise instructions for identifying the plurality of features for estimating subject cell source fraction as a subset of the plurality of bins. Each respective bin in the subset of the plurality of bins satisfies a selection criterion based on the corresponding measure of association for the respective bin.

Another aspect of the present disclosure provides the above-disclosed computing system where the one or more programs further comprise instructions for performing any of the methods disclosed herein alone or in combination.

Another aspect of the present disclosure provides a non-transitory computer readable storage medium storing one or more programs for estimating cell source fraction for a subject. The one or more programs are configured for execution by a computer. The one or more programs comprise instructions for obtaining a training dataset, in electronic form. The training dataset comprises, for each respective training subject in a plurality of training subjects: a) a corresponding methylation pattern of each respective cell-free fragment in a corresponding training plurality of cell-free fragments, and b) a subject cancer indication of the respective training subject. The corresponding methylation pattern of each respective cell-free fragment (i) is determined by a methylation sequencing of one or more nucleic acid samples comprising the respective fragment in a corresponding biological sample obtained from the respective training subject, and (ii) comprises a methylation state of each CpG site in a corresponding plurality of CpG sites in the respective fragment. The subject cancer condition is one of a first cancer condition and a second cancer condition. The one or more programs comprise instructions for mapping each cell-free fragment in each plurality of cell-free fragments to a bin in a plurality of bins. Here, each respective bin in the plurality of bins represents a corresponding portion of a human reference genome, thereby obtaining a plurality of training sets of cell-free fragments, and each training set of cell-free fragments is mapped to a different bin in the plurality of bins. The one or more programs further comprise instructions for assigning a cell-free fragment cancer condition to each respective cell-free fragment in each training set of cell-free fragments in the plurality of training sets of cell-free fragments as a function of an output of a classifier upon inputting a methylation pattern of the respective cell-free fragment into the classifier. The cell-free fragment cancer condition is one of the first cancer condition and the second cancer condition. The one or more programs further comprise instructions for determining, for each respective bin in the plurality of bins, a corresponding measure of association I between (a) the subject cancer condition of respective training subjects in the plurality of training subjects and (b) the cell-free fragment cancer condition of respective cell-free fragments in the corresponding training set of cell-free fragments mapping to the respective bin. The one or more programs comprise instructions for identifying the plurality of features for estimating subject cell source fraction as a subset of the plurality of bins. Each respective bin in the subset of the plurality of bins satisfies a selection criterion based on the corresponding measure of association for the respective bin.

Another aspect of the present disclosure provides the above-disclosed non-transitory computer readable storage medium in which the one or more programs further comprise instructions for performing any of the methods disclosed herein alone or in combination.

B. Embodiments Directed to Determining Cell Source Fraction for a Test Subject Using Methylation Data Acquired from Cell-Free DNA.

Another aspect of the present disclosure provides for estimating cell source fraction for a subject. The method comprises, at a computer system having one or more processors, and memory storing one or more programs for execution by the one or more processors, obtaining, in electronic form, a corresponding methylation pattern of each respective cell-free fragment in a plurality of cell-free fragments. Here the corresponding methylation pattern of each respective cell-free fragment (i) is determined by a methylation sequencing of one or more nucleic acid samples comprising the respective fragment in a biological sample obtained from the subject and (ii) comprises a methylation state of each CpG site in a corresponding plurality of CpG sites in the respective fragment. The method comprises mapping each cell-free fragment in the plurality of cell-free fragments to a bin in a plurality of bins, thereby obtaining a plurality of sets of cell-free fragments. Each set of cell-free fragments mapped to a different bin in the plurality of bin. The method also comprises assigning a cell-free fragment cancer condition to each respective cell-free fragment in each set of cell-free fragments in the plurality of sets of cell-free fragments, as a function of an output of a classifier upon inputting a methylation pattern of the respective cell-free fragment into the classifier. The cell-free fragment cancer condition is one of the first cancer condition and the second cancer condition. The method continues by computing a first measure of central tendency of the number of cell-free fragments from the subject that have been assigned the first cancer condition in each set of cell-free fragments across the plurality of bins, and computing a second measure of central tendency of the number of cell-free fragments from the subject in each set of cell-free fragments across the plurality of bins. The method further comprises estimating the cell source fraction for the subject using the first measure of central tendency and the second measure of central tendency.

In some embodiments, the plurality of bins consists of between 1000 bins. In some embodiments, the plurality of bins consists of between 15,000 bins and 80,000 bins.

In some embodiments, each respective bin in the plurality of bins has, on average, between 10 and 1200 residues. In some embodiments, each respective bin in the plurality of bins has, on average, between 10 and 10000 residues.

In some embodiments, the first measure of central tendency is an arithmetic mean, a weighted mean, a midrange, a midhinge, a trimean, a Winsorized mean, a mean, or a mode of the number of cell-free fragments from the subject that have been assigned the first cancer condition in each set of cell-free fragments across the plurality of bins. In some embodiments, the second measure of central tendency is an arithmetic mean, a weighted mean, a midrange, a midhinge, a trimean, a Winsorized mean, a mean, or a mode of the number of cell-free fragments from the subject in each set of cell-free fragments across the plurality of bins.

In some embodiments, estimating the cell source fraction comprises dividing the first measure of central tendency by the second measure of central tendency.

In some embodiments, the methylation sequencing is paired-end sequencing. In some embodiments, the methylation sequencing is single-read sequencing.

In some embodiments, the plurality of cell-free fragments has an average length of less than 500 nucleotides.

In some embodiments, the first cancer condition is cancer and the second cancer condition is absence of cancer.

In some embodiments, the first cancer condition is one of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, or leukemia, and the second cancer condition is absence of cancer.

In some embodiments, the first cancer condition is one of a stage of adrenal cancer, a stage of biliary tract cancer, a stage of bladder cancer, a stage of bone/bone marrow cancer, a stage of brain cancer, a stage of breast cancer, a stage of cervical cancer, a stage of colorectal cancer, a stage of cancer of the esophagus, a stage of gastric cancer, a stage of head/neck cancer, a stage of hepatobiliary cancer, a stage of kidney cancer, a stage of liver cancer, a stage of lung cancer, a stage of ovarian cancer, a stage of pancreatic cancer, a stage of pelvis cancer, a stage of pleura cancer, a stage of prostate cancer, a stage of renal cancer, a stage of skin cancer, a stage of stomach cancer, a stage of testis cancer, a stage of thymus cancer, a stage of thyroid cancer, a stage of uterine cancer, a stage of lymphoma, a stage of melanoma, a stage of multiple myeloma, or a stage of leukemia, and the second cancer condition is absence of cancer.

In some embodiments, the methylation sequencing is whole genome methylation sequencing. In some embodiments, the methylation sequencing is targeted sequencing using a plurality of nucleic acid probes and each respective bin in the plurality of bins is associated with at least one corresponding nucleic acid probe in the plurality of nucleic acid probes.

In some embodiments, the plurality of nucleic acid probes comprises 1,000 or more nucleic acid probes, 2,000 or more nucleic acid probes, 3,000 or more nucleic acid probes, 5,000 or more nucleic acid probes, 10,000 or more nucleic acid probes or between 1,000 nucleic acid probes and 30,000 nucleic acid probes.

In some embodiments, each bin in the plurality of bins comprises 2, 3, 4, 5, 6, 7, 8, 9, 10, 11, 12, 13, 14, 15, 16, 17, 18, 19, 20 or more CpG sites. In some embodiments, each bin in the plurality the bins comprises 2, 3, 4, 5, 6, 7, 8, 9, 10, 11, 12, 13, 14, 15, 16, 17, 18, 19, 20 or more contiguous CpG sites. In some embodiments, each bin in the plurality of bins consists of between 2 and 100 contiguous CpG sites in a human reference genome.

In some embodiments, the biological sample is a liquid biological sample. In some embodiments, the biological sample is a blood sample. In some embodiments, the biological sample comprises blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject.

In some embodiments, the methylation state of a respective CpG site in the corresponding plurality of CpG sites in the respective fragment is: methylated when the respective CpG site is determined by the methylation sequencing to be methylated, unmethylated when the respective CpG site is determined by the methylation sequencing to not be methylated, and flagged as “other” when the methylation sequencing is unable to call the methylation state of the respective CpG site as methylation or unmethylated.

In some embodiments, the methylation sequencing detects one or more 5-methylcytosine (5mC) and/or 5-hydroxymethylcytosine (5hmC) in the respective fragment.

In some embodiments, the methylation sequencing comprises conversion of one or more unmethylated cytosines or one or more methylated cytosines, in sequence reads of the respective fragment, to a corresponding one or more uracils. In some embodiments, the one or more uracils are detected during the methylation sequencing as one or more corresponding thymines. In some embodiments, the conversion of one or more unmethylated cytosines or one or more methylated cytosines comprises a chemical conversion, an enzymatic conversion, or combinations thereof.

In some embodiments, the first model is a first mixture model comprising a first plurality of sub-models, the second model is a second mixture model comprising a second plurality of sub-models, and each sub-model in the first and second plurality of sub-models represents an independent corresponding methylation model for a source of cell-free fragments in the corresponding biological sample.

In some embodiments, each independent corresponding methylation model is one of a binomial model, beta-binomial model, independent sites model or Markov model.

In some embodiments, two or more sub-models in the first plurality of sub-models are independent sites models, and two or more sub-models in the second plurality of sub-models are independent sites models.

In some embodiments, the method further comprises applying one or more filter conditions to the plurality of cell-free fragments.

In some embodiments, a filter condition in the one or more filter conditions is application of a p-value threshold to the corresponding methylation pattern for each respective cell-free fragment in the plurality of cell-free fragments, where the p-value threshold is representative of how frequently a methylation pattern is observed in a cohort of non-cancer subjects.

In some embodiments, the p-value threshold is between 0.001 and 0.20. In some embodiments, the p-value threshold is between 0.01 and 0.10. In some embodiments the p-value threshold is greater than 0.001, 0.005, 0.010, 0.020, 0.030, 0.040, 0.050, 0.060, 0.070, 0.080, 0.090, or 0.010.

In some embodiments, the cohort comprises at least twenty, at least thirty, at least 50, at least 100, at least 500, or at least 1000 subjects. In some embodiments, the plurality of cell-free fragments comprises at least 300, at least 500, at least 1000, at least 5000, at least 8,000, or at least 10,000 different corresponding methylation patterns.

In some embodiments, the p-value threshold is satisfied for a methylation pattern from the subject when the corresponding methylation pattern for each respective cell-free fragment in the plurality of cell-free fragments has a p-value of 0.10 or less, 0.05 or less, or 0.01 or less.

In some embodiments, a filter condition in the one or more filter conditions is application of a requirement that each respective cell-free fragment in the plurality of cell-free fragments is represented by a threshold number of sequence reads in a corresponding plurality of sequence reads measured from the one or more nucleic acid samples comprising the respective fragment in the corresponding biological sample. In some embodiments, the threshold number is 2, 3, 4, 5, 6, 7, 8, 9, 10, or an integer between 10 and 100.

In some embodiments, a filter condition in the one or more filter conditions is application of a requirement that each respective cell-free fragment in the plurality of cell-free fragments is represented by a threshold number of cell-free nucleic acids in the one or more nucleic acid samples comprising the respective fragment in the corresponding biological sample. In some embodiments, the threshold number is 2, 3, 4, 5, 6, 7, 8, 9, 10, or an integer between 10 and 100.

In some embodiments, a filter condition in the one or more filter conditions is application of a requirement that each respective cell-free fragment in the plurality of cell-free fragments have a threshold number of CpG sites. In some embodiments, the threshold number of CpG sites is at least 1, 2, 3, 4, 5, 6, 7, 8, 9 or 10 CpG sites.

In some embodiments, a filter condition in the one or more filter conditions is a requirement that each respective cell-free fragment in the plurality of cell-free fragments have a length of less than a threshold number of base pairs. In some embodiments, the threshold number of base pairs is one thousand, two thousand, three thousand, or four thousand contiguous base pairs in length.

In some embodiments, a single filter condition is applied. In some embodiments, two filter conditions are applied. In some embodiments, three filter conditions are applied. In some embodiments, four filter conditions are applied.

In some embodiments, the method further comprises repeating the obtaining, mapping, assigning, computing the first and second measure of central tendency, and estimating the cell source fraction for the test subject at each respective time point in a plurality of time points across an epoch, thus obtaining a corresponding cell source fraction, in a plurality of cell source fractions, for the test subject at each respective time point. In some embodiments this plurality of cell source fractions is used to determine a state or progression of a disease condition in the test subject during the epoch in the form of an increase or decrease of a first cell source fraction over the epoch.

In some embodiments, each epoch is a period of months and each time point in the plurality of time points is a different time point in the period of months. In some embodiments, the period of months is less than four months. In some embodiments, each epoch is one month long. In some embodiments, each epoch is two months long. In some embodiments, each epoch is three months long. In some embodiments, each epoch is four months long. In some embodiments, each epoch is five, six, seven, eight, nine, ten, eleven, twelve, thirteen, fourteen, fifteen, sixteen, seventeen, eighteen, nineteen, twenty, twenty-one, twenty-two, twenty-three or twenty-four months long.

In some embodiments, the epoch is a period of years and each time point in the plurality of time points is a different time point in the period of years. In some embodiments, the period of years is between one year and ten years. In some embodiments, the period of years is one year, two years, three years, four years, five years, six years, seven years, eight years, nine years, or ten years. In some embodiment the epoch is between one and thirty years.

In some embodiments, the epoch is a period of hours and each time point in the plurality of time points is a different time point in the period of hours. In some embodiments, the period of hours is between one hour and twenty-four hours. In some embodiments, the period of hours is 1, 2, 3, 4, 5, 6, 7, 8, 9, 10, 11, 12, 13, 14, 15, 16, 17, 18, 19, 20, 21, 22, 23, or 24 hours.

In some embodiments, the method further comprises changing a diagnosis of the test subject when the first cell source fraction of the subject is observed to change by a threshold amount across the epoch. For instance, in some embodiments, the diagnosis is changed from having cancer to being in remission. As another example, in some embodiments, the diagnosis is changed from not having cancer to having cancer. As another example, in some embodiments, the diagnosis is changed from having a first stage of a cancer to having a second stage of a cancer. As another example, in some embodiments, the diagnosis is changed from having a second stage of a cancer to having a third stage of a cancer. As still another example, in some embodiments, the diagnosis is changed from having a third stage of a cancer to having a fourth stage of a cancer. As still another example, in some embodiments, the diagnosis is changed from having a cancer that has not metastasized to having a cancer that has metastasized.

In some embodiments, the method further comprises changing a prognosis of the test subject when the first cell source fraction of the subject is observed to change by a threshold amount across the epoch. For example, in some embodiments, the prognosis involves life expectancy and the prognosis is changed from a first life expectancy to a second life expectancy, where the first and second life expectancy differ in their duration. In some embodiments, the change in prognosis increases the life expectancy of the subject. In some embodiments, the change in prognosis decreases the life expectancy of the subject.

In some embodiments, the method further comprises changing a treatment of the test subject when the first cell source fraction of the subject is observed to change by a threshold amount across the epoch. In some embodiments, the changing of the treatment comprises initiating a cancer medication, increasing the dosage of a cancer medication, stopping a cancer medication, or decreasing the dosage of the cancer medication. In some embodiments, the changing of the treatment comprises initiating or terminating treatment of the subject with Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof. In some embodiments, the changing of the treatment comprises increasing or decreasing a dosage of Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof administered to the subject. In some embodiments, the threshold is greater than ten percent, greater than twenty percent, greater than thirty percent, greater than forty percent, greater than fifty percent, greater than two-fold, greater than three-fold, or greater than five-fold.

In some embodiments, the tumor fraction for the test subject is between 0.003 and 1.0. In some embodiments, the tumor fraction for the test subject is between 0.005 and 0.80. In some embodiments, the tumor fraction for the test subject is between 0.01 and 0.70. In some embodiments, the tumor fraction for the test subject is between 0.05 and 0.60.

In some embodiments, the method further comprises applying a treatment regimen to the test subject based at least in part, on a value of the cell source fraction for the test subject. In some embodiments, the treatment regimen comprises applying an agent for cancer to the test subject. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the method further comprises using the cell source fraction for the test subject to evaluate a response of the subject to the agent for cancer. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or a generic equivalent thereof.

In some embodiments, the test subject has been treated with an agent for cancer and the method further comprises using the cell source fraction for the test subject to determine whether to intensify or discontinue the agent for cancer in the test subject. For instance, in some embodiments, observation of at least a threshold cell source fraction (e.g., greater than 0.05, 0.10, 0.15, 0.20, 0.25, or 0.30, etc.) is used as a basis for intensifying (e.g., increasing the dosage, increasing radiation level in radiation treatment) of the agent for cancer in the test subject. In some embodiments, observation of less than a threshold cell source fraction (e.g., less than 0.05, 0.10, 0.15, 0.20, 0.25, or 0.30, etc.) is used as a basis for discontinuing use of the agent for cancer in the test subject.

In some embodiments, the test subject has been subjected to a surgical intervention to address the cancer and the method further comprises using the cell source fraction for the test subject to evaluate a condition of the test subject in response to the surgical intervention. In some embodiments the condition is a metric based upon calculated cell source fraction using the methods provided in the present disclosure.

In some embodiments, a bin in the plurality of bins corresponds to a single genomic region listed in one or more of Tables 1-24 of International Patent Application No. PCT/US2019/025358 (published as WO2019/195268A2), lists 1-8 of International Patent Application No. PCT/US2019/053509 (published as WO2020/069350A1), and/or lists 1-16 of International Patent Application No. PCT/US2020/015082 (published as WO2020/154682A2), each of which is hereby incorporated herein by reference in its entirety.

In some embodiments, a bin in the plurality of bins corresponds to a combination of genomic region listed in one or more of Tables 1-24 of International Patent Application No. PCT/US2019/025358 (published as WO2019/195268A2), lists 1-8 of International Patent Application No. PCT/US2019/053509 (published as WO2020/069350A1), and/or lists 1-16 of International Patent Application No. PCT/US2020/015082 (published as WO2020/154682A2), each of which is hereby incorporated herein by reference in its entirety, each of which is hereby incorporated by reference. For instance, in some embodiments a bin in the plurality of bins includes one, two, three, four, five, or more than five regions listed in Tables 1-24 of International Patent Publication No. WO2019/195268A2, lists 1-8 of International Patent Publication No. WO2020/069350A1, and/or lists 1-16 of International Patent Publication No. WO2020/154682A2.

In some embodiments, a bin in the plurality of bins maps to at least 30%, 40%, 50%, 60%, 70%, 80%, 90%, 95%, 99% or 100% of a genomic region listed in one or more of Tables 1-24 of International Patent Publication No. WO2019/195268A2, lists 1-8 of International Patent Publication No. WO2020/069350A1, and/or lists 1-16 of International Patent Publication No. WO2020/154682A2.

In some embodiments, a bin in the plurality of bins maps to at least between 50 and 95% of a genomic region listed in one or more of Tables 1-24 of International Patent Publication No. WO2019/195268A2, lists 1-8 of International Patent Publication No. WO2020/069350A1, and/or lists 1-16 of International Patent Publication No. WO2020/154682A2.

In some embodiments, a bin in the plurality of bins maps to between one and 10 unique corresponding genomic regions in one or more of Tables 1-24 of International Patent Publication No. WO2019/195268A2, lists 1-8 of International Patent Publication No. WO2020/069350A1, and/or and lists 1-16 of International Patent Publication No. WO2020/154682A2.

In some embodiments, each bin in the plurality of bins maps to a single unique corresponding genomic region in one or more of Tables 1-24 of International Patent Publication No. WO2019/195268A2, lists 1-8 of International Patent Publication No. WO2020/069350A1, and/or lists 1-16 of International Patent Publication No. WO2020/154682A2.

In some embodiments, the plurality of cell-free fragments, for a respective subject, comprises at least 10,000, 15,000, 20,000, 25,000, 50,000, 100,000, 200,000, 300,000, 500,000 or 1 million cell-free fragments. In some embodiments, the plurality of cell-free fragments, for a respective subject, comprises at least 1 million cell-free fragments.

In some embodiments, each bin in the plurality of bins comprises less than 100 nucleic acid residues, less than 500 nucleic acid residues, less than 1000 nucleic acid residues, less than 2500 nucleic acid residues, less than 5000 nucleic acid residues, less than 10,000 nucleic acid residues, less than 25,000 nucleic acid residues, less than 50,000 nucleic acid residues, less than 100,000 nucleic acid residues, less than 250,000 nucleic acid residues, or less than 500,000 nucleic acid residues.

In some embodiments, each bin in the plurality of bins comprises between (i) 100 nucleic acid residues and (ii) 500, 1000, 2500, 5000, 10,000, 25,000, 50,000, 100,000, 250,000, or 500,000 nucleic acid residues.

Another aspect of the present disclosure provides a computing system for estimating cell source fraction of a subject. The computing system comprises one or more processors and memory storing one or more programs to be executed by the one or more processor. The one or more programs comprises instructions for obtaining, in electronic form, a corresponding methylation pattern of each respective cell-free fragment in a plurality of cell-free fragments. Here the corresponding methylation pattern of each respective cell-free fragment (i) is determined by a methylation sequencing of one or more nucleic acid samples comprising the respective fragment in a biological sample obtained from the subject and (ii) comprises a methylation state of each CpG site in a corresponding plurality of CpG sites in the respective fragment. The one or more programs further comprise instructions for mapping each cell-free fragment in the plurality of cell-free fragments to a bin in a plurality of bins, thereby obtaining a plurality of sets of cell-free fragments. Each set of cell-free fragments mapped to a different bin in the plurality of bin. The one or more programs further comprise instructions for assigning a cell-free fragment cancer condition to each respective cell-free fragment in each set of cell-free fragments in the plurality of sets of cell-free fragments. The cell-free fragment cancer condition is one of the first cancer condition and the second cancer condition, as a function of an output of a classifier upon inputting a methylation pattern of the respective cell-free fragment into the classifier. The one or more programs further comprise instructions for computing a first measure of central tendency of the number of cell-free fragments from the subject that have been assigned the first cancer condition in each set of cell-free fragments across the plurality of bins, and computing a second measure of central tendency of the number of cell-free fragments from the subject in each set of cell-free fragments across the plurality of bins. The one or more programs further comprise instructions for estimating the cell source fraction for the subject using the first measure of central tendency and the second measure of central tendency.

Another aspect of the present disclosure provides the above-disclosed computing system where the one or more programs further comprise instructions for performing any of the methods disclosed above alone or in combination.

Another aspect of the present disclosure provides a non-transitory computer readable storage medium storing one or more programs for estimating cell source fraction for a subject. The one or more programs are configured for execution by a computer. The one or more programs comprise instructions for obtaining, in electronic form, a corresponding methylation pattern of each respective cell-free fragment in a plurality of cell-free fragments. The corresponding methylation pattern of each respective cell-free fragment (i) is determined by a methylation sequencing of one or more nucleic acid samples comprising the respective fragment in a biological sample obtained from the subject, and (ii) comprises a methylation state of each CpG site in a corresponding plurality of CpG sites in the respective fragment. The one or more programs comprise instructions for mapping each cell-free fragment in the plurality of cell-free fragments to a bin in a plurality of bins, thereby obtaining a plurality of sets of cell-free fragments. Here each set of cell-free fragments is mapped to a different bin in the plurality of bins. The one or more programs further comprise instructions for assigning a cell-free fragment cancer condition to each respective cell-free fragment in each set of cell-free fragments in the plurality of sets of cell-free fragments as a function of an output of a classifier upon inputting a methylation pattern of the respective cell-free fragment into the classifier. The cell-free fragment cancer condition is one of the first cancer condition and the second cancer condition. The one or more programs further comprise instructions for computing a first measure of central tendency of the number of cell-free fragments from the subject that have been assigned the first cancer condition in each set of cell-free fragments across the plurality of bins and computing a second measure of central tendency of the number of cell-free fragments from the subject in each set of cell-free fragments across the plurality of bins. The one or more programs comprise instructions for estimating the cell source fraction for the subject using the first measure of central tendency and the second measure of central tendency.

Another aspect of the present disclosure provides the above-disclosed non-transitory computer readable storage medium in which the one or more programs further comprise instructions for performing any of the above-disclosed methods alone or in combination.

Various embodiments of systems, methods and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an example block diagram illustrating a computing device in accordance with some embodiments of the present disclosure.

FIGS. 2A and 2B collectively illustrate an example flowchart of a method of identifying a plurality of features for estimating subject cell source fraction, in which dashed boxes represent optional steps, in accordance with some embodiments of the present disclosure.

FIGS. 3A and 3B collectively illustrate an example flowchart of a method of estimating cell source fraction for a subject, in which dashed boxes represent optional steps, in accordance with some embodiments of the present disclosure.

FIG. 4 illustrates a plot of the ctDNA fraction of subjects with any of the listed cancers, as a function of cancer stage in accordance with some embodiments of the present disclosure.

FIG. 5 illustrates a flowchart of a method for preparing a nucleic acid sample for sequencing in accordance with some embodiments of the present disclosure.

FIG. 6 illustrates a graphical representation of the process for obtaining sequence reads in accordance with some embodiments of the present disclosure.

FIG. 7 illustrates a comparison of tumor fraction estimates based on whole-genome bisulfite sequencing data with known tumor fraction derived from tissue-based whole-genome sequencing data, in accordance with some embodiments of the present disclosure. In particular, the WGBS estimated tumor fraction comprises the ratio of the mean number of abnormal fragments with the average total number of fragments (e.g., where each fragment is mapped to a particular bin or region of a reference genome). FIG. 7 is based on the sequencing information from 495 subjects. At known tissue tumor fraction >0.01, the Spearman correlation for the WGBS tumor fraction estimation is 0.86. At known tissue tumor fraction >0.005, the Spearman correlation for the WGBS tumor fraction estimation is 0.90. At known tissue tumor fraction >0.001, the Spearman correlation for the WGBS tumor fraction estimation is 0.89. At known tissue tumor fraction >0.0001, the Spearman correlation for the WGBS tumor fraction estimation is 0.74. This demonstrates that WGBS-based estimates of tumor fraction are correlated with known tissue tumor fractions.

FIG. 8 illustrates a measure of mutual information that is used in accordance with some embodiments of the present disclosure for feature identification.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for determining an estimated cell source fraction of a subject. In an example embodiment, nucleic acid fragments are obtained from a biological sample of a subject. The biological sample comprises cell-free nucleic acid. Thus, the nucleic acid fragments are cell-free nucleic acid. The nucleic acid fragments are evaluated for methylation status for a predefined set of methylation sites, and are each assigned a score based on methylation state. The plurality of methylation state scores is transformed into a plurality of counts, which are compared to a corresponding methylation score for each methylation site in the predefined set of methylation sites. The corresponding methylation scores are from analysis of methylation patterns in a cell source. This comparison determines a frequency of methylation in the subject, which is then used to estimate cell source fraction, with regard to the cell source.

### Definitions

As used herein, the term “about” or “approximately” mean within an acceptable error range for the particular value as determined by one of ordinary skill in the art, which depends in part on how the value is measured or determined, e.g., the limitations of the measurement system. For example, in some embodiments “about” mean within 1 or more than 1 standard deviation, per the practice in the art. In some embodiments, “about” means a range of ±20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. In some embodiments, the term “about” refers to ±10%. In some embodiments, the term “about” refers to ±5%.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the terms “biological sample,” “patient sample,” and “sample” are interchangeably used and refer to any sample taken from a subject, which can reflect a biological state associated with the subject. In some embodiments such samples contain cell-free nucleic acids such as cell-free DNA. In some embodiments, such samples include nucleic acids other than or in addition to cell-free nucleic acids. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

In some embodiments, a biological sample is derived from one tissue type (e.g., from a single organ such as breast, lung, prostate, colorectal, renal, uterine, pancreatic, esophageal, lymph, ovarian, cervical, epidermal, thyroid, bladder, or gastric). In some embodiments, a biological sample is derived from a two or more tissue types (e.g., a combination of tissue from two or more organs). In some embodiments, a biological sample is derived from one or more cell types (e.g., cells originating from a single organ or from a predetermined set of organs).

As disclosed herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), ribonucleic acid (RNA, e.g., message RNA (mRNA), short inhibitory RNA (siRNA), ribosomal RNA (rRNA), transfer RNA (tRNA), microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of RNA or DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. For RNA, the base cytosine is replaced with uracil and the sugar 2′ position includes a hydroxyl moiety. A nucleic acid may be prepared using a nucleic acid obtained from a subject as a template.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells Cell-free nucleic acids are used interchangeably as circulating nucleic acids. Examples of the cell-free nucleic acids include but are not limited to RNA, mitochondrial DNA, or genomic DNA. As used herein, the terms “cell-free nucleic acid,” “cell-free DNA,” and “cfDNA” are used interchangeably. As used herein, the term “circulating tumor DNA” or “ctDNA” refers to nucleic acid fragments that originate from tumor cells or other types of cancer cells, which may be released into a fluid from an individual's body (e.g., bloodstream) as result of biological processes such as apoptosis or necrosis of dying cells or actively released by viable tumor cells. Examples of the cell-free nucleic acids include but are not limited to RNA, mitochondrial DNA, or genomic DNA.

As disclosed herein, the term “circulating tumor DNA” or “ctDNA” refers to nucleic acid fragments that originate from aberrant tissue, such as the cells of a tumor or other types of cancer, which may be released into a subject's bloodstream as result of biological processes such as apoptosis or necrosis of dying cells or actively released by viable tumor cells.

As disclosed herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of genomic sequence. In some embodiments, a method can include analysis of multiple mapped nucleic acid fragments to a plurality of genomic regions. Genomic regions can be approximately the same length or the genomic sections can be different lengths. In some embodiments, genomic regions are of about equal length. In some embodiments genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome or all or part of two or more chromosomes. In some embodiments, genomic regions may span one, two, or more entire chromosomes. In addition, the genomic regions may span joint or disjointed portions of multiple chromosomes.

As used herein, the term “fragment” is used interchangeably with “nucleic acid fragment” (e.g., a DNA fragment), and refers to a portion of a polynucleotide or polypeptide sequence that comprises at least three consecutive nucleotides. In the context of sequencing of cell-free nucleic acid molecules found in a biological sample, the terms “fragment” and “nucleic acid fragment” interchangeably refer to a cell-free nucleic acid molecule that is found in the biological sample or a representation thereof. In such a context, sequencing data (e.g., sequence reads from whole genome sequencing, targeted sequencing, etc.) are used to derive one or more copies of all or a portion of such a nucleic acid fragment. Such sequence reads, which in fact may be obtained from sequencing of PCR duplicates of the original nucleic acid fragment, therefore “represent” or “support” the nucleic acid fragment. There may be a plurality of sequence reads that each represent or support a particular nucleic acid fragment in the biological sample (e.g., PCR duplicates). In some embodiments, nucleic acid fragments can be considered cell-free nucleic acids. In some embodiments, sequence reads from PCR duplicates can be misleading; for example, when the abundance level of a particular cell-free nucleic acid molecule needs to be determined. In such embodiments, only one copy of a nucleic acid fragment is used to represent the original cell-free nucleic acid molecule (e.g., duplicates are removed through molecular identifiers that are attached to the cell-free nucleic acid molecule during the library preparation process). In some embodiments, methylation sequencing data can be used to further distinguish these nucleic acid fragments. For example, two nucleic acid fragments that share identical or near identical sequences may still correspond to different original cell-free nucleic acid molecules if they each harbor a different methylation pattern.

In some embodiments, two fragments are considered to share near identical nucleic acid sequences when the respective fragment sequences differ from each other by fewer than 2 nucleotides, by fewer than 3 nucleotides, by fewer than 4 nucleotides, by fewer than 5 nucleotides, by fewer than 6 nucleotides, by fewer than 7 nucleotides, by fewer than 8 nucleotides, by fewer than 9 nucleotides, by fewer than 10 nucleotides, by fewer than 15 nucleotides, by fewer than 20 nucleotides, by fewer than 25 nucleotides, by fewer than 30 nucleotides, by fewer than 35 nucleotides, by fewer than 40 nucleotides, by fewer than 45 nucleotides, or by fewer than 50 nucleotides. In some embodiments, two fragments are considered to share near identical sequences when the respective fragment sequences differ from each other by less than 1% of the total nucleotides, by less than 2% of the total nucleotides, by less than 3% of the total nucleotides, by less than 4% of the total nucleotides, or by less than 5% of the total nucleotides.

In some embodiments, a first fragment from a respective (e.g., a first or second) plurality of nucleic acid fragments is aligned to a first location in a reference genome and a second fragment from the respective (e.g., the first or second) plurality of nucleic acid fragments is aligned to a second location in a reference genome. In some embodiments, the first location and the second location correspond to distinct regions in the reference genome. In some embodiments, the first and second locations are a same location (e.g., the first and second locations correspond to a same region of the reference genome). In some embodiments, the first and second locations overlap in the reference genome by at least 1 residue, at least 2 residues, at least 3 residues, at least 4 residues, at least 5 residues, at least 6 residues, at least 7 residues, at least 8 residues, at least 9 residues, at least 10 residues, by at least 11 residues, by at least 12 residues, by at least 13 residues, by at least 14 residues, by at least 15 residues, by at least 16 residues, by at least 17 residues, by at least 18 residues, by at least 19 residues, by at least 20 residues, by at least 30 residues, by at least 40 residues, by at least 50 residues, by at least 60 residues, by at least 70 residues, by at least 80 residues, by at least 90 residues, or by at least 100 residues. In some embodiments, the first location and the second location overlap in the reference genome by between 1 and 50 residues.

In some embodiments, a respective fragment is mapped to at least a first location and a second location of a reference genome (e.g., the nucleic acid sequence corresponding to the respective fragment is present in at least two different locations in the reference genome). In some embodiments, a respective fragment is mapped to at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 locations of a reference genome. In some embodiments, the at least two mapped locations of the reference genome are separated from each other in the reference genome by at least 1 residue, at least 5 residues, at least 10 residues, at least 25 residues, at least 50 residues, at least 100 residues, at least 200 residues, at least 300 residues, at least 400 residues, at least 500 residues, at least 600 residues, at least 700 residues, at least 800 residues, at least 900 residues, or at least 1000 residues. In some embodiments, the at least two mapped locations comprise different genes in the reference genome. In some embodiments, the at least two mapped locations are located on different chromosomes of the reference genome.

A nucleic acid fragment can retain the biological activity and/or some characteristics of the parent polynucleotide. In an example, nasopharyngeal cancer cells can deposit fragments of Epstein-Barr Virus (EBV) DNA into the bloodstream of a subject, e.g., a patient. These fragments can comprise one or more BamHI-W sequence fragments, which can be used to detect the level of tumor-derived DNA in the plasma. The BamHI-W sequence fragment corresponds to a sequence that can be recognized and/or digested using the Bam-HI restriction enzyme. The BamHI-W sequence can refer to the sequence 5′-GGATCC-3′.

In addition, a polynucleotide, for example, can be broken up, or fragmented into, a plurality of segments, either through natural processes, as is the case with, e.g., cfDNA fragments that can naturally occur within a biological sample, or through in vitro manipulation. Various methods of fragmenting nucleic acids are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N. Y. (2001) (“Sambrook et al.) which is incorporated herein by reference for all purposes. These methods can be optimized to digest a nucleic acid into fragments of a selected size range.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As disclosed herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As disclosed herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

As used herein a “methylome” can be a measure of an amount of DNA methylation at a plurality of sites or loci in a genome. The methylome can correspond to all of a genome, a substantial part of a genome, or relatively small portion(s) of a genome. A “tumor methylome” can be a methylome of a tumor of a subject (e.g., a human). A tumor methylome can be determined using tumor tissue or cell-free tumor DNA in plasma. A tumor methylome can be one example of a methylome of interest. A methylome of interest can be a methylome of an organ that can contribute nucleic acid, e.g., DNA into a bodily fluid (e.g., a methylome of brain cells, a bone, lungs, heart, muscles, kidneys, etc.). The organ can be a transplanted organ.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of nucleic acid fragments showing methylation at the site over the total number of nucleic acid fragments covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by nucleic acid fragments mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region only includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

As used herein, a “plasma methylome” can be the methylome determined from plasma or serum of an animal (e.g., a human). A plasma methylome can be an example of a cell-free methylome since plasma and serum can include cell-free DNA. A plasma methylome can be an example of a mixed methylome since it can be a mixture of tumor/patient methylome. A “cellular methylome” can be a methylome determined from cells (e.g., blood cells or tumor cells) of a subject, e.g., a patient. A methylome of blood cells can be called a blood cell methylome (or blood methylome).

As used herein, the term “abnormal methylation pattern” or “anomalous methylation pattern” refers to a methylation state vector, methylation pattern, or a methylation status of a DNA molecule having the methylation state vector that is expected to be found in a sample less frequently than a threshold value. In a particular embodiment provided herein, the expectedness of finding a specific methylation state vector in a healthy control group comprising healthy individuals is represented by a p-value. In some embodiments, p-values of methylation state vectors are determined as described in Example 5 of PCT/US2020/034317, entitled “Systems and Methods for Determining Whether a Subject has a Cancer Condition Using Transfer Learning,” filed on May 22, 2020, and in U.S. patent application Ser. No. 16/352,602, entitled “Anomalous fragment detection and classification,” filed Mar. 13, 2019, now published as US2019/0287652, each of which is incorporated by reference herein in its entirety. A low p-value score, thereby, generally corresponds to a methylation state vector that is relatively unexpected in comparison to other methylation state vectors within samples from healthy individuals in the healthy control group. A high p-value score generally corresponds to a methylation state vector that is relatively more expected in comparison to other methylation state vectors found in samples from healthy individuals in the healthy control group. A methylation state vector having a p-value lower than a threshold value (e.g., 0.1, 0.01, 0.001, 0.0001, etc.) can be defined as an abnormal methylation pattern. Various methods known in the art can be used to calculate a p-value or expectedness of a methylation pattern or a methylation state vector. Exemplary methods provided herein involve use of a Markov chain probability that assumes methylation statuses of CpG sites to be dependent on methylation statuses of neighboring CpG sites. Alternate methods provided herein calculate the expectedness of observing a specific methylation state vector in healthy individuals by utilizing a mixture-model including multiple mixture components, each being an independent-sites model where methylation at each CpG site is assumed to be independent of methylation statuses at other CpG sites. Methods provided herein use genomic regions having an anomalous methylation pattern. A genomic region can be determined to have an anomalous methylation pattern when cfDNA fragments corresponding to or originated from the genomic region have methylation state vectors that appear less frequently than a threshold value in reference samples. The reference samples can be samples from control subjects or healthy subjects. The frequency for a methylation state vector to appear in the reference samples can be represented as a p-value score. When cfDNA fragments corresponding to or originated from the genomic region do not have a single, uniform methylation state vector, the genomic region can have multiple p-value scores for multiple methylation state vectors. In this case, the multiple p-value scores can be summed or averaged before being compared to the threshold value. Various methods known in the art can be adopted to compare p-value scores corresponding to the genomic region and the threshold value, including but not limited to arithmetic mean, geometric mean, harmonic mean, median, mode, etc.

As used herein, the term “relative abundance” can refer to a ratio of a first amount of nucleic acid fragments having a particular characteristic (e.g., a specified length, ending at one or more specified coordinates/ending positions, aligning to a particular region of the genome, or having a particular methylation status) to a second amount nucleic acid fragments having a particular characteristic (e.g., a specified length, ending at one or more specified coordinates/ending positions, or aligning to a particular region of the genome). In one example, relative abundance may refer to a ratio of the number of DNA fragments ending at a first set of genomic positions to the number of DNA fragments ending at a second set of genomic positions. In some aspects, a “relative abundance” can be a type of separation value that relates an amount (one value) of cell-free DNA molecules ending within one window of genomic position to an amount (other value) of cell-free DNA molecules ending within another window of genomic positions. The two windows can overlap, but can be of different sizes. In other embodiments, the two windows cannot overlap. Further, in some embodiments, the windows are of a width of one nucleotide, and therefore are equivalent to one genomic position.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites”. In other instances, methylation may occur at a cytosine not part of a CpG site or at another nucleotide other than cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer.

Various challenges arise in the identification of anomalously methylated cfDNA fragments. First, determining a subject's cfDNA to be anomalously methylated only holds weight in comparison with a group of control subjects, such that if the control group is small in number, the determination loses confidence with the small control group. Additionally, among a group of control subjects' methylation status can vary which can be difficult to account for when determining a subject's cfDNA to be anomalously methylated. On another note, methylation of a cytosine at a CpG site causally influences methylation at a subsequent CpG site.

Those of skill in the art will appreciate that the principles described herein are equally applicable for the detection of methylation in a non-CpG context, including non-cytosine methylation.

As disclosed herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. The terms “subject” and “patient” are used interchangeably herein and refer to a human or non-human animal who is known to have, or potentially has, a medical condition or disorder, such as, e.g., a cancer. In some embodiments, a subject is a male or female of any stage (e.g., a man, a woman or a child).

A subject from whom a sample is taken, or is treated by any of the methods or compositions described herein can be of any age and can be an adult, infant or child. In some cases, the subject, e.g., patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old). A particular class of subjects, e.g., patients that can benefit from a method of the present disclosure is subjects, e.g., patients over the age of 40.

Another particular class of subjects, e.g., patients that can benefit from a method of the present disclosure is pediatric patients, who can be at higher risk of chronic heart symptoms. Furthermore, a subject, e.g., patient from whom a sample is taken, or is treated by any of the methods or compositions described herein, can be male or female.

The term “normalize” as used herein means transforming a value or a set of values to a common frame of reference for comparison purposes. For example, when a diagnostic ctDNA level is “normalized” with a baseline ctDNA level, the diagnostic ctDNA level is compared to the baseline ctDNA level so that the amount by which the diagnostic ctDNA level differs from the baseline ctDNA level can be determined.

As used herein the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the term “level of cancer” refers to whether cancer exists (e.g., presence or absence), a stage of a cancer, a size of tumor, presence or absence of metastasis, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In one embodiment, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer.

The terms “cancer load,” “tumor load,” “cancer burden” and “tumor burden” are used interchangeably herein to refer to a concentration or presence of tumor-derived nucleic acids in a test sample. As such, the terms “cancer load,” “tumor load,” “cancer burden” and “tumor burden” are non-limiting examples of a cell source fraction or tumor fraction in a biological sample. In some embodiments, tumor fraction is a specific version of cell source fraction.

As used herein, the term “tissue” corresponds to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein the term “untrained classifier” refers to a classifier that has not been trained on a target dataset. However, an untrained classifier may be partially training on a primary dataset (e.g., a small and/or reference dataset). It will be appreciated that the term “untrained classifier” does not exclude the possibility that transfer learning techniques are used in such training of the untrained classifier. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained classifier is provided with additional data over and beyond that of the primary training dataset. Typically, this additional data is in the form of coefficients (e.g., regression coefficients) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained classifier in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a classifier such as regression to the first auxiliary training dataset) may be applied to the second auxiliary training dataset using transfer learning techniques (e.g., the above described two-dimensional matrix multiplication), which in turn may result in a trained intermediate classifier whose coefficients are then applied to the primary training dataset and this, in conjunction with the primary training dataset itself, is applied to the untrained classifier. Alternatively, a first set of coefficients learned from the first auxiliary training dataset (by application of a classifier such as regression to the first auxiliary training dataset) and a second set of coefficients learned from the second auxiliary training dataset (by application of a classifier such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In either example, knowledge regarding cell source (e.g., cancer type, etc.) derived from the first and second auxiliary training datasets is used, in conjunction with the cell source labeled primary training dataset), to train the untrained classifier.

The term “classification” can refer to any number(s) or other characters(s) that are associated with a particular property of a sample. For example, a “+” symbol (or the word “positive”) can signify that a sample is classified as having deletions or amplifications. In another example, the term “classification” refers to an amount of tumor tissue in the subject and/or sample, a size of the tumor in the subject and/or sample, a stage of the tumor in the subject, a tumor load in the subject and/or sample, and presence of tumor metastasis in the subject. In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff size refers to a size above which fragments are excluded. In some embodiments, a threshold value is a value above or below which a particular classification applies. Either of these terms can be used in either of these contexts.

As used herein, the term “cancer-associated changes” or “cancer-specific changes” can include cancer-derived mutations (including single nucleotide mutations, deletions or insertions of nucleotides, deletions of genetic or chromosomal segments, translocations, inversions), amplification of genes, virus-associated sequences (e.g., viral episomes, viral insertions, viral DNA that is infected into a cell and subsequently released by the cell, and circulating or cell-free viral DNA), aberrant methylation profiles or tumor-specific methylation signatures, aberrant cell-free nucleic acid (e.g., DNA) size profiles, aberrant histone modification marks and other epigenetic modifications, and locations of the ends of cell-free DNA fragments that are cancer-associated or cancer-specific.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragments obtained from a sample from the subject. A reference genome can refer to a haploid or diploid genome to which nucleic acid fragments from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Exemplary System Embodiments

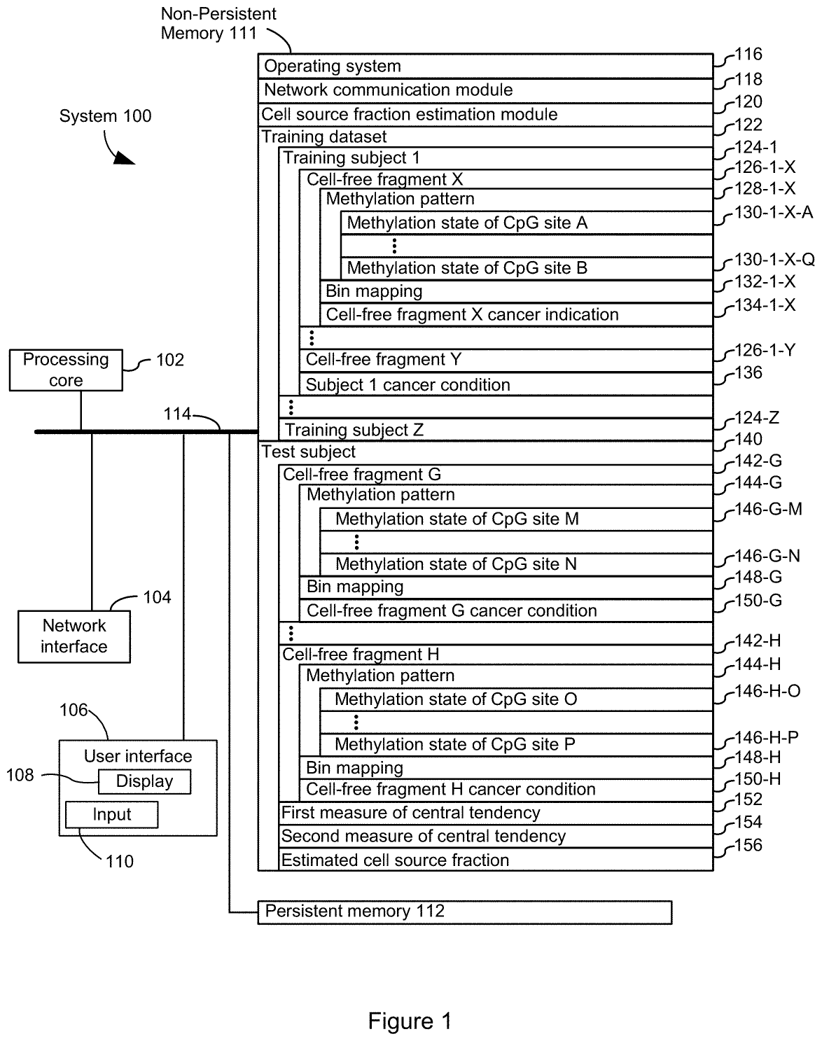

Details of an exemplary system are now described in conjunction with FIG. 1. FIG. 1 is a block diagram illustrating system 100 in accordance with some implementations. Device 100 in some implementations includes one or more processing units CPU(s) 102 (also referred to as processors or processing core), one or more network interfaces 104, user interface 106, non-persistent memory 111, persistent memory 112, and one or more communication buses 114 for interconnecting these components. One or more communication buses 114 optionally include circuitry (sometimes called a chipset) that interconnects and controls communications between system components. Non-persistent memory 111 typically includes high-speed random access memory, such as DRAM, SRAM, DDR RAM, ROM, EEPROM, flash memory, whereas persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Persistent memory 112 optionally includes one or more storage devices remotely located from the CPU(s) 102. Persistent memory 112, and the non-volatile memory device(s) within non-persistent memory 112, comprise non-transitory computer readable storage medium. In some implementations, non-persistent memory 111 or alternatively non-transitory computer readable storage medium stores the following programs, modules and data structures, or a subset thereof, sometimes in conjunction with persistent memory 112:
optional operating system 116, which includes procedures for handling various basic system services and for performing hardware dependent tasks;
optional network communication module (or instructions) 118 for connecting the system 100 with other devices, or a communication network;
a cell source fraction estimation module 120 for determining a cell source fraction 158 of a test subject 140 in a biological sample of the test subject;
a training dataset 122 that comprises, for each respective training subject 124 (e.g., 124-1, . . . , 124-Z, where Z is a positive integer greater than 1), for each respective cell-free fragment 126 (e.g., 126-1-X, . . . , 126-1-Y, where X and Y are any positive integers with Y greater than X) of the respective training subject at least (i) a corresponding methylation pattern 128 (e.g., 128-1-X) that is determined from at least the respective methylation state of each CpG site 130 (e.g., 130-1-X-A, 130-1-X-Q) in the respective cell-free fragment; and (ii) a corresponding subject cancer indication of the respective training subject 136.
a test subject dataset 140 that comprises, for each cell-free fragment 142 (e.g., 142-G, . . . , 142-H, where G and H are positive integers with H greater than G) in a plurality of cell-free fragments derived from a biological sample of the test subject, (i) a respective methylation pattern 144 (e.g., 144-G, . . . , 144-H) that is determined from at least the respective methylation state of each CpG site 148 (e.g., 146-G-M, 146-G-N, . . . , 146-H-O, . . . 146 H-P, where M, N, O and P are positive integers) in the respective cell-free fragment, (ii) a respective bin mapping 148 (e.g., 148-G, . . . , 148H), and (iii) a respective predicted cell-free fragment cancer condition 150 (e.g., 150-G, . . . , 150-H), the test subject dataset further comprises a first measure of central tendency 152, a second measure of central tendency 154, and an estimated cell source fraction 156.

In accordance with the present disclosure, a corresponding bin mapping 132 (e.g., 132-1-X) of each respective cell-free fragment and an assignment of a cell-free fragment cancer condition 134 (e.g., 134-1-X) of each respective cell-free fragment is made. For convenience and ease of interpretation, these data constructs are shown as being in the training dataset. However, in typical embodiments, such data constructs are calculated from the methylation patterns of the cell-free fragments in the training set and are not part of the original dataset. In other embodiments, the bin mapping 132 and cell-free fragment cancer conditions are part of the training dataset 122 that are obtained.

In accordance with some implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as functional description of the various features which may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these datasets and/or modules may be in persistent memory 112.

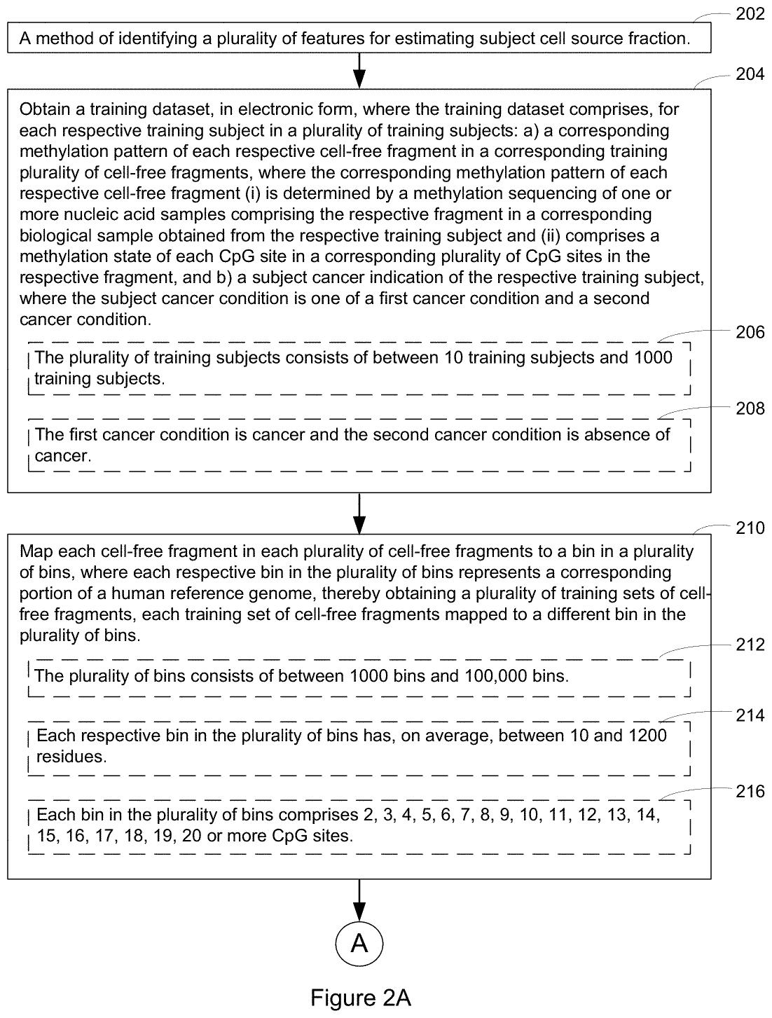

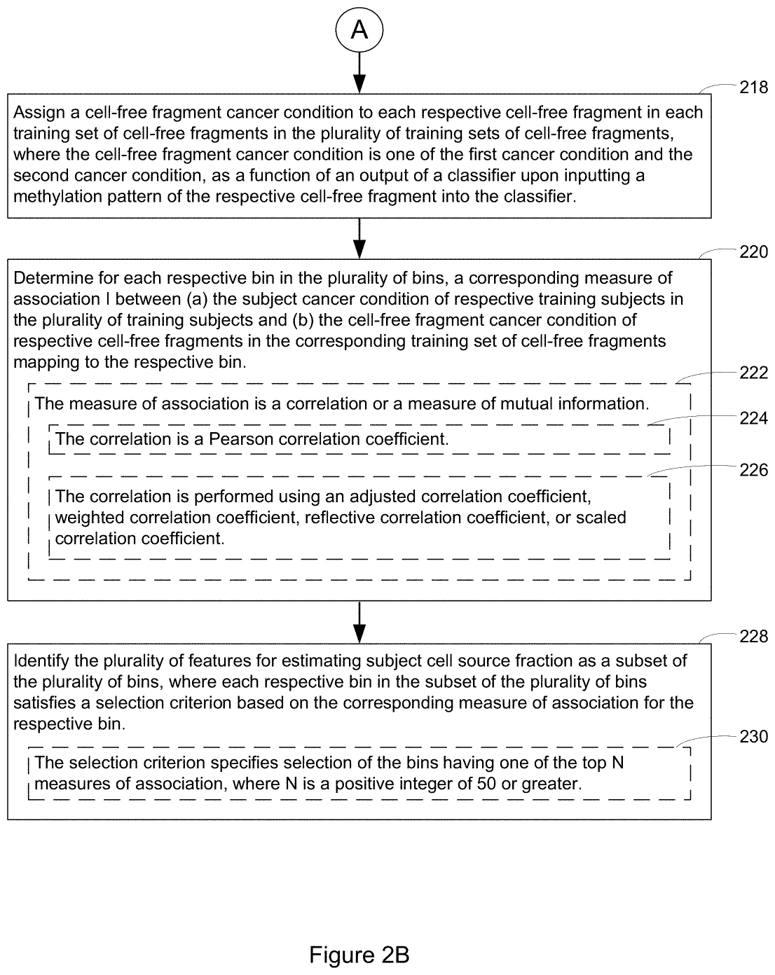

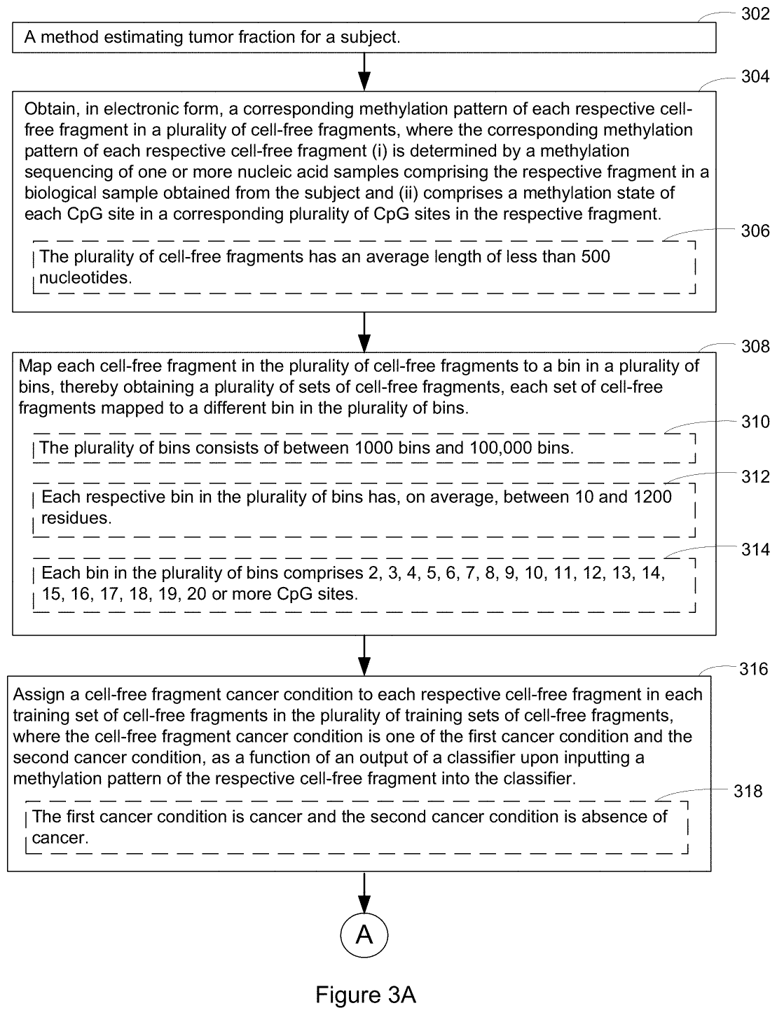

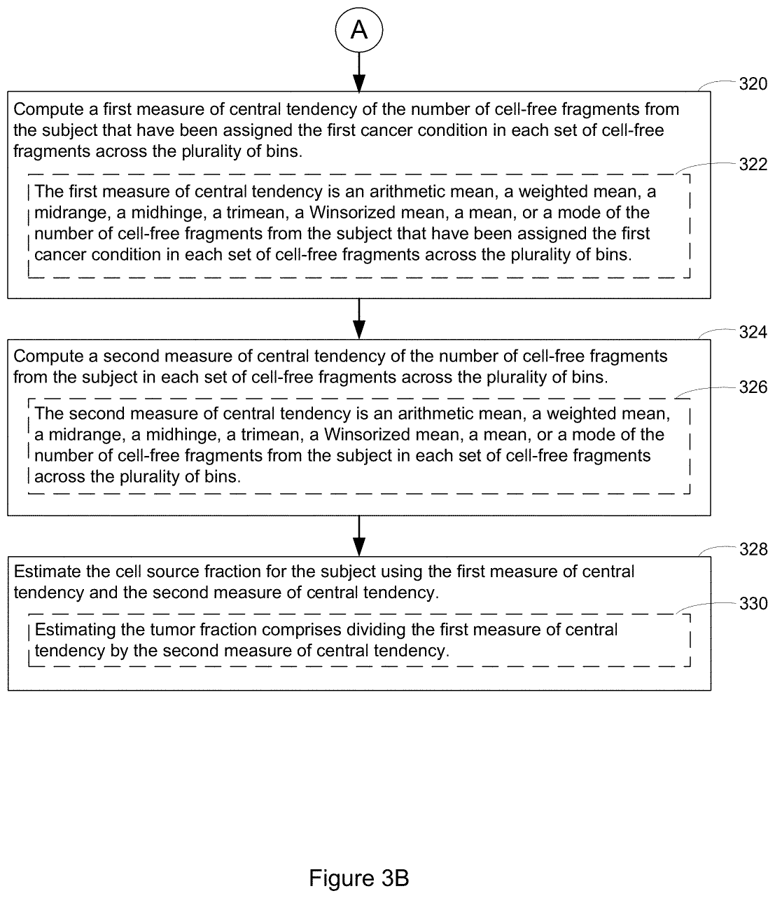

While a system in accordance with the present disclosure has been disclosed with reference to FIG. 1, methods in accordance with the present disclosure are now detailed with reference to FIGS. 2A and 2B and 3A and 3B. It will be appreciated that any of the disclosed methods can make use of or work in conjunction with any of the assays or algorithms disclosed in U.S. patent application Ser. No. 15/793,830, filed Oct. 25, 2017 and/or International Patent Publication No. PCT/US17/58099, having an International Filing Date of Oct. 24, 2017, each of which is hereby incorporated by reference, in order to determine a cancer condition in a test subject or a likelihood that the subject has the cancer condition.

Identifying features for estimating cell source fraction.

Block 202. One aspect of the present disclosure provides a method of identifying a plurality of features for estimating cell source fraction for a subject that is performed at a computer system having one or more processors, and memory storing one or more programs for execution by the one or more processors.

In some embodiments, the cell source fraction of Block 202 of FIG. 2A corresponds to a first cancer condition of a common primary site of origin. In some embodiments, the cell source fraction corresponds to a tumor fraction of a certain cancer type, or a fraction thereof. In some embodiments, the cell source fraction corresponds to a tumor fraction of a predetermined stage of a first cancer condition. In some embodiments, the cell source fraction is derived from one or more types of human cells.

Subjects and Cancer Conditions.

Block 204. In Block 204 of FIG. 2A, the method proceeds by obtaining a training dataset in electronic form. The training dataset comprises, for each training subject in a plurality of training subjects, at least a) a corresponding methylation pattern of each respective cell-free fragment in a corresponding training plurality of cell-free fragments, and b) a subject cancer indication of the respective training subject, where the subject cancer condition is one of a first cancer condition and a second cancer condition.

In accordance with Block 206, in some embodiments, the plurality of training subjects consists of between 10 and 1000 training subjects. In some embodiments, the plurality of training subjects consists of at least 10 training subjects, at least 25 training subjects, at least 50 training subjects, at least 100 training subjects, at least 250 training subjects, at least 500 training subjects, at least 750 training subjects, at least 1000 training subjects or at least 1500 training subjects. In some embodiments, the plurality of training subjects comprises between 10 and 100,000 training subjects, between 100 and 50,000 training subjects or between 100 and 10,000 training subjects.

In some embodiments, there is a balanced number of training subjects having the first cancer condition and the second cancer condition in the plurality of training subjects (e.g., the plurality of training subjects comprises a substantially similar number of training subjects with each subject cancer condition). For example, if the plurality of training subjects comprises at least 50 training subjects with the first cancer condition, the plurality of training subjects also comprises at least 50 training subjects with the second cancer condition, or if the plurality of training subjects comprises at least 500 training subjects with the first cancer condition, the plurality of training subjects also comprises at least 500 training subjects with the second cancer condition. In some embodiments, between 5 percent and 95 percent of the training subjects have the first cancer condition while the remainder have the second cancer condition. In some embodiments, between 20 percent and 80 percent of the training subjects have the first cancer condition while the remainder have the second cancer condition. In some embodiments, between 30 percent and 70 percent of the training subjects have the first cancer condition while the remainder have the second cancer condition. In some embodiments, between 40 percent and 60 percent of the training subjects have the first cancer condition while the remainder have the second cancer condition. In some embodiments, between 45 percent and 55 percent of the training subjects have the first cancer condition while the remainder have the second cancer condition.

Referring to Block 208, in some embodiments, the first cancer condition consists of cancer and the second cancer condition is absence of cancer. In some embodiments, the first cancer condition is one of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, or leukemia, and the second cancer condition is absence of cancer. In some embodiments, the first cancer condition is one of a stage of adrenal cancer, a stage of biliary tract cancer, a stage of bladder cancer, a stage of bone/bone marrow cancer, a stage of brain cancer, a stage of breast cancer, a stage of cervical cancer, a stage of colorectal cancer, a stage of cancer of the esophagus, a stage of gastric cancer, a stage of head/neck cancer, a stage of hepatobiliary cancer, a stage of kidney cancer, a stage of liver cancer, a stage of lung cancer, a stage of ovarian cancer, a stage of pancreatic cancer, a stage of pelvis cancer, a stage of pleura cancer, a stage of prostate cancer, a stage of renal cancer, a stage of skin cancer, a stage of stomach cancer, a stage of testis cancer, a stage of thymus cancer, a stage of thyroid cancer, a stage of uterine cancer, a stage of lymphoma, a stage of melanoma, a stage of multiple myeloma, or a stage of leukemia, and the second cancer condition is absence of cancer.

In some embodiments, the second cancer condition is one of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, or leukemia. In some embodiments, the second cancer condition is one of a stage of adrenal cancer, a stage of biliary tract cancer, a stage of bladder cancer, a stage of bone/bone marrow cancer, a stage of brain cancer, a stage of breast cancer, a stage of cervical cancer, a stage of colorectal cancer, a stage of cancer of the esophagus, a stage of gastric cancer, a stage of head/neck cancer, a stage of hepatobiliary cancer, a stage of kidney cancer, a stage of liver cancer, a stage of lung cancer, a stage of ovarian cancer, a stage of pancreatic cancer, a stage of pelvis cancer, a stage of pleura cancer, a stage of prostate cancer, a stage of renal cancer, a stage of skin cancer, a stage of stomach cancer, a stage of testis cancer, a stage of thymus cancer, a stage of thyroid cancer, a stage of uterine cancer, a stage of lymphoma, a stage of melanoma, a stage of multiple myeloma, or a stage of leukemia.

In some embodiments, the subject cancer condition is one of a first cancer condition, a second cancer condition, and a third cancer condition. In some embodiments, the respective subject cancer condition for each training subject in the plurality of training subjects is individually selected from a plurality of cancer conditions. In some such embodiments, the plurality of training subjects comprises at least a minimum number of training subjects with each respective cancer condition in the plurality of cancer conditions. In some embodiments, the minimum number of training subjects with each respective cancer condition is at least 10, at least 20, at least 30, at least 40, at least 50, at least 60, at least 70, at least 80, at least 90, at least 100, at least 150, at least 200, at least 250, at least 300, at least 350, at least 400, at least 450, or at least 500 training subjects.

In some embodiments, the plurality of cancer conditions comprises at least 5, at least 10, or at least 20 unique cancer conditions. In some embodiments, the plurality of cancer conditions consists of 22 unique cancer conditions.

In some embodiments, each cancer condition in the plurality of cancer conditions is one of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, or leukemia. In some embodiments, each cancer condition in the plurality of cancer conditions is one of a stage of adrenal cancer, a stage of biliary tract cancer, a stage of bladder cancer, a stage of bone/bone marrow cancer, a stage of brain cancer, a stage of breast cancer, a stage of cervical cancer, a stage of colorectal cancer, a stage of cancer of the esophagus, a stage of gastric cancer, a stage of head/neck cancer, a stage of hepatobiliary cancer, a stage of kidney cancer, a stage of liver cancer, a stage of lung cancer, a stage of ovarian cancer, a stage of pancreatic cancer, a stage of pelvis cancer, a stage of pleura cancer, a stage of prostate cancer, a stage of renal cancer, a stage of skin cancer, a stage of stomach cancer, a stage of testis cancer, a stage of thymus cancer, a stage of thyroid cancer, a stage of uterine cancer, a stage of lymphoma, a stage of melanoma, a stage of multiple myeloma, or a stage of leukemia.

Obtaining Cell-Free Fragments and Methylation Sequencing.

Referring again to Block 204, the corresponding methylation pattern of each respective cell-free fragment, in each corresponding training plurality of cell-free fragments, for each training subject (i) is determined by a methylation sequencing of one or more nucleic acid samples comprising the respective fragment in a corresponding biological sample obtained from the respective training subject, and (ii) comprises a methylation state of each CpG site in a corresponding plurality of CpG sites in the respective fragment.

In some embodiments, the corresponding biological sample is a liquid biological sample. In some embodiments, the corresponding biological sample is a blood sample. In some embodiments, the corresponding biological sample comprises blood, whole blood, plasma, serum, urine, cerebrospinal fluid, fecal, saliva, sweat, tears, pleural fluid, pericardial fluid, or peritoneal fluid of the training subject. In some embodiments, the corresponding biological sample consists of blood, whole blood, plasma, serum, urine, cerebrospinal fluid, fecal, saliva, sweat, tears, pleural fluid, pericardial fluid, or peritoneal fluid of the training subject.

In some embodiments, the one or more nucleic acid samples in the corresponding biological sample from the training subject is a cell-free nucleic acid sample (e.g., obtained from a liquid biological sample). In some embodiments, the cell-free nucleic acids that are obtained from a biological sample are any form of nucleic acid defined in the present disclosure, or a combination thereof. For example, in some embodiments, the cell-free nucleic acid that is obtained from a biological sample is a mixture of RNA and DNA.

In some embodiments, where the corresponding training plurality of cell-free fragments for a respective training subject is derived from cell-free nucleic acids from a biological sample (e.g., a liquid biological sample), it is advantageous that the cell-free nucleic acids exhibit an appreciable cell source fraction. In some embodiments, the cell source fraction, with respect to the first or second cancer condition, for the corresponding training subject is at least two percent, at least five percent, at least ten percent, at least fifteen percent, at least twenty percent, at least twenty-five percent, at least fifty percent, at least seventy-five percent, at least ninety percent, at least ninety-five percent, or at least ninety-eight percent.

In some embodiments, the biological sample is processed to extract the cell-free nucleic acids in preparation for sequencing analysis. By way of a non-limiting example, in some embodiments, cell-free nucleic acid fragments are extracted from a biological sample (e.g., blood sample) collected from a subject in K2 EDTA tubes. In the case where the biological samples are blood, the samples are processed within two hours of collection by double spinning of the biological sample first at ten minutes at 1000 g, and then the resulting plasma is spun ten minutes at 2000 g. The plasma is then stored in 1 ml aliquots at −80° C. In this way, a suitable amount of plasma (e.g., 1-5 ml) is prepared from the biological sample for the purposes of cell-free nucleic acid extraction. In some such embodiments cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton, et al., 2017, “Phylogenetic ctDNA analysis depicts early stage lung cancer evolution,” Nature, 545(7655): 446-451, which is hereby incorporated by reference. Other equivalent methods can be used to prepare cell-free nucleic acid from biological methods for the purpose of sequencing, and all such methods are within the scope of the present disclosure.

In some embodiments, the cell-free nucleic acid fragments are treated to convert unmethylated cytosines to uracils. In one embodiment, the method uses a bisulfite treatment of the DNA that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cell-free nucleic acid fragments, a sequencing library is prepared. Optionally, the sequencing library is enriched for cell-free nucleic acid fragments, or genomic regions, that are informative for cell origin using a plurality of hybridization probes. The hybridization probes are short oligonucleotides that hybridize to particularly specified cell-free nucleic acid fragments, or targeted regions, and enrich for those fragments or regions for subsequent sequencing and analysis. In some embodiments, hybridization probes are used to perform a targeted, high-depth analysis of a set of specified CpG sites that are informative for cell origin. Once prepared, the sequencing library or a portion thereof is sequenced to obtain a plurality of sequence reads.

In some embodiments, sequence reads obtained from a biological sample of a subject are normalized relative a reference set (e.g., as obtained from a plurality of reference subjects such as a control cohort of healthy subjects). U.S. Patent Publication No. 2019-0287649, entitled “Method and System for Selecting, Managing, and Analyzing Data of High Dimensionality,” published Sep. 19, 2019, which is hereby incorporated by reference herein in its entirety, discloses multiple methods of normalization.

In some embodiments, the plurality of sequence reads comprises at least 100, at least 500, at least 1000, at least 2000, at least 3000, at least 4000, at least 5000, at least 6000, at least 7000, at least 8000, at least 9000, at least 10,000, at least 20,000, at least 50,000, at least 100,000, or at least one million sequence reads. In some embodiments, the plurality of sequence reads comprises at least 5 million, at least 10 million, or at least 100 million sequence reads.

In some embodiments, the training plurality of cell-free fragments, for a respective training subject in the plurality of training subjects comprises at least 100, at least 500, at least 1000, at least 2000, at least 3000, at least 4000, at least 5000, at least 6000, at least 7000, at least 8000, at least 9000, at least 10,000, at least 20,000, at least 50,000, at least 100,000, at least one million, at least five million, or at least ten million cell-free fragments. In some embodiments, the training plurality of cell-free fragments, for each respective training subject in the plurality of training subjects comprises at least 100, at least 500, at least 1000, at least 2000, at least 3000, at least 4000, at least 5000, at least 6000, at least 7000, at least 8000, at least 9000, at least 10,000, at least 20,000, at least 50,000, at least 100,000, at least one million, at least five million, or at least ten million cell-free fragments.

In some embodiments, a first training subject in the plurality of training subjects has a first corresponding plurality of cell-free fragments comprising a first number of cell-free fragments, and a second training subject in the plurality of training subjects has a second corresponding plurality of cell-free fragments comprising a second number of cell-free fragments that is different from the first number (e.g., in some embodiments, each training subject has a different training plurality of cell-free fragments).

In some embodiments, each corresponding training plurality of cell-free fragments has an average length of less than 500 nucleotides. In some embodiments, each corresponding training plurality of cell-free fragments have an average length of less than 100, 200, 300, 400, 500, 600, 700, 800, 900, or 1000 nucleotides.

In some embodiments, the sequencing comprises methylation sequencing.

In some embodiments, the methylation sequencing detects one or more 5-methylcytosine (5mC) and/or 5-hydroxymethylcytsine (5hmC) in the respective fragment. In some such embodiments, the methylation sequencing further comprises conversion of one or more unmethylated cytosines or one or more methylated cytosines, in sequence reads of the respective fragment, to a corresponding one or more uracils. In some embodiments, the one or more uracils are detected during the methylation sequencing as one or more corresponding thymines. In some embodiments, the conversion of one or more unmethylated cytosines or one or more methylated cytosines comprises a chemical conversion, an enzymatic conversion, or combinations thereof. In some embodiments, cytosine conversion is performed as described in U.S. Patent Application No. 62/877,755, entitled “Systems and Methods for Determining Tumor Fraction” and filed on Jul. 23, 2019, which is hereby incorporated by reference.

In some embodiments, the methylation state of a respective CpG site in the corresponding plurality of CpG sites in the respective fragment is: (i) methylated when the respective CpG site is determined by the methylation sequencing to be methylated, (ii) unmethylated when the respective CpG site is determined by the methylation sequencing to not be methylated, and/or (iii) flagged as “other” when the methylation sequencing is unable to call the methylation state of the respective CpG site as methylation or unmethylated.

In some embodiments, the methylation sequencing (e.g., used to determine methylation patterns) is paired-end sequencing. In some embodiments, the methylation sequencing is single-read sequencing. In some embodiments, the methylation sequencing is whole genome methylation sequencing (e.g., whole genome bisulfate sequencing).

A whole genome sequencing assay refers to a physical assay that generates sequence reads for a whole genome or a substantial portion of the whole genome that can be used to determine large variations such as copy number variations or copy number aberrations. Such a physical assay may employ whole genome sequencing techniques or whole exome sequencing techniques.

In some embodiments, the whole genome methylation sequencing identifies one or more methylation state vectors as described, for example, in U.S. patent application Ser. No. 16/352,602, entitled “Anomalous fragment detection and classification,” filed Mar. 13, 2019, now published as US2019/0287652, which is hereby incorporated by reference herein in its entirety.

In some embodiments, the sequencing comprises any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, the sequencing-by-ligation platform from Applied Biosystems, the ION TORRENT technology from Life technologies, and/or nanopore sequencing. In some embodiments, the sequencing comprises sequencing-by-synthesis and reversible terminator-based sequencing (e.g., Illumina's Genome Analyzer; Genome Analyzer II; HISEQ 2000; HISEQ 2500 (Illumina, San Diego Calif.)).

In some embodiments, the whole genome methylation sequencing is used to sequence a portion of the genome. In some embodiments the portion of the genome is at least 10 percent, 20 percent, 30 percent, 40 percent, 50 percent, 60 percent, 70 percent, 80 percent, 90 percent, 95 percent, 99 percent, 99.9 percent or all of a genome (e.g., a human reference genome). In some embodiments, the whole genome methylation sequencing generates a plurality of sequence reads, where each sequence read in the plurality of sequence reads has a sequence length of 1000 base pairs or less. In some embodiments, the whole genome methylation sequencing obtains a sequencing coverage of the portion of the genome that is at least 5×, at least 10×, at least 15×, at least 20×, at least 25×, at least 30×, at least 50×, at least 100×, or at least 200× across the portion of the genome. In some embodiments, the whole genome methylation sequencing obtains a sequencing coverage of at least 5×, at least 10×, at least 15×, at least 20×, at least 25×, at least 30×, at least 50×, at least 100×, or at least 200× across the entire genome.

In some embodiments, the methylation sequencing is targeted sequencing using a plurality of nucleic acid probes and each bin (e.g., genomic region of interest) in the plurality of bins is associated with at least one nucleic acid probe in the plurality of nucleic acid probes.

In some embodiments, the targeted sequencing targets portions of a genome (e.g., a human reference genome) using the plurality of nucleic acid probes, and the targeted sequencing obtains a sequencing coverage of at least 5×, at least 10×, at least 15×, at least 20×, at least 25×, at least 30×, at least 50×, at least 100×, at least 250×, at least 500×, or at least 1000× of the targeted portions of the genome (e.g., to which the probes map). In some embodiments, the targeted sequencing obtains a sequencing coverage of at least 100×, at least 200×, at least 500×, at least 1,000×, at least 2,000×, at least 3,000×, at least 4,000×, at least 5,000×, at least 10,000×, at least 15,000×, at least 20,000×, at least 25,000×, at least 30,000×, at least 40,000×, or at least 50,000× across selected regions in the genome of the subject.

In some embodiments, targeted panel sequencing is beneficial because it obtains significant information about regions of interest in the reference genome of the subject while being more efficient (e.g., with regard to use of materials for sequencing, length of time required for sequencing, etc.) than whole genome sequencing, for example. In other words, in some embodiments, targeted panel sequencing serves to obtain as much information as possible from the underlying data (e.g., at both the cell-free nucleic acid level and across genomic regions) while making the problem of determining tumor fraction (and/or tumor origin) for the subject computationally tractable. For example, a reference genome (e.g., a human reference genome) includes approximately 28 million CpG sites, while a targeted methylation panel directed to the reference genome includes fewer CpG sites (e.g., between 10,000 and 5 million CpG sites, between 100,000 and 3 million CpG sites, etc.

In some embodiments, at least one probe in the plurality of probes is designed to bind and enrich nucleic acids in the biological sample that contain at least one predetermined CpG site. In some implementations, each probe in the plurality of probes is designed to bind and enrich nucleic acids in the biological sample that contain at least one predetermined CpG site.

In some embodiments, each probe in the plurality of probes is designed for targeting nucleic acids that have a certain number of predetermined CpG sites. For example, in some embodiments, one or more probes in the plurality of probes is designed to bind and enrich nucleic acids in the biological sample that contain 50 or fewer predetermined CpG sites, 40 or fewer predetermined CpG sites, 30 or fewer predetermined CpG sites, 25 or fewer predetermined CpG sites, 22 or fewer predetermined CpG sites, 20 or fewer predetermined CpG sites, 18 or fewer predetermined CpG sites, 15 or fewer predetermined CpG sites, 12 or fewer predetermined CpG sites, 10 or fewer predetermined CpG sites, 5 or fewer predetermined CpG sites, 3 or fewer predetermined CpG sites.

In some embodiments, for targeted methylation sequencing, the plurality of probes comprises between 1,000 and 2,000,000 probes. In some embodiments, the plurality of probes comprises 1,000 or more probes, 2,000 or more probes, 3,000 or more probes, 4,000 or more probes, 5,000 or more probes, 10,000 or more probes, 20,000 or more probes or 30,000 or more probes. In some embodiments, the plurality of probes is between 1,000 and 30,000 probes. In some embodiments, the plurality of probes comprises at least 5,000, at least 10,000, at least 20,000, at least 30,000, at least 40,000, at least 50,000, at least 100,000, at least 200,000, at least 300,000, at least 400,000, at least 500,000, at least 600,000, at least 700,000, at least 800,000, at least 900,000, or at least 1,000,000 probes.

It should be appreciated that the plurality of probes may include other number of probes, non-limiting examples of which include 1,500,000 probes or fewer, 1,400,000 probes or fewer, 1,300,000 probes or fewer, 1,200,000 probes or fewer, 1,100,000 probes or fewer, 1,000,000 probes or fewer, 900,000 probes or fewer, 800,000 probes or fewer, 700,000 probes or fewer, 600,000 probes or fewer, 500,000 probes or fewer, 400,000 probes or fewer, 300,000 probes or fewer, 200,000 probes or fewer, 100,000 probes or fewer, 90,000 probes or fewer, 80,000 probes or fewer, 70,000 probes or fewer, 60,000 probes or fewer, 50,000 probes or fewer, 40,000 probes or fewer, 30,000 probes or fewer, 20,000 probes or fewer, 10,000 probes or fewer, 9,000 probes or fewer, 8,000 probes or fewer, 7,000 probes or fewer, 6,000 probes or fewer, 5,000 probes or fewer, 4,000 probes or fewer, 4,000 probes or fewer, 2,000 probes or fewer, or 1,000 probes or fewer.

In some embodiments, the plurality of probes target a plurality of genetic targets (e.g., portions of the reference genome and/or a panel of gene targets) that collectively covers 0.5 to 50 megabases of the reference genome. In some embodiments, the plurality of genetic targets of the plurality of probes collectively covers 5 to 40 megabases of the reference genome, 10 to 30 megabases of the reference genome, 15 to 35 megabases of the reference genome, 20 to 30 megabases of the reference genome, 25 to 35 megabases of the reference genome, or 30 to 40 megabases of the reference genome.

In some embodiments, the plurality of probes is a targeted cancer assay panel. A number of targeted cancer assay panels are known in the art, for example, as described in International Patent Application No. PCT/US2019/025358, published as WO2019/195268A2, entitled “Methylated Markers and Targeted Methylation Probe Panels,” filed Apr. 2, 2019, International Patent Application No. PCT/US2019/053509, published as WO2020/069350A1, entitled “Methylated Markers and Targeted Methylation Probe Panel,” filed Sep. 27, 2019, and International Patent Application No. PCT/US2020/015082, published as WO2020/154682A2, entitled “Detecting Cancer, Cancer Tissue or Origin, or Cancer Type,” filed Jan. 24, 2020, each of which is hereby incorporated by reference herein in its entirety. For example, in some embodiments, a targeted cancer assay panel comprises a plurality of probes (or probe pairs) that can capture fragments (cell-free nucleic acids) that can together provide information relevant to determination of tumor fraction and/or diagnosis of cancer. In some embodiments, a plurality of probes in a targeted cancer assay panel includes at least 50, 100, 500, 1,000, 2,000, 2,500, 5,000, 6,000, 7,500, 10,000, 15,000, 20,000, 25,000, or 50,000 pairs of probes. In other embodiments, a plurality of probes in a targeted cancer assay panel includes at least 500, 1,000, 2,000, 5,000, 10,000, 12,000, 15,000, 20,000, 30,000, 40,000, 50,000, or 100,000 probes. In some embodiments, the plurality of probes collectively comprise at least 0.1 million, 0.2 million, 0.4 million, 0.6 million, 0.8 million, 1 million, 2 million, 3 million, 4 million, 5 million, 6 million, 7 million, 8 million, 9 million, or 10 million nucleotides. In some embodiments, the probes (or probe pairs) are specifically designed to target one or more genomic regions differentially methylated in cancer and non-cancer samples.

For example, a plurality of probes in a targeted cancer assay panel can include probes that can selectively bind and enrich cfDNA fragments that are differentially methylated in cancerous samples. In this case, sequencing of the enriched fragments can provide information relevant to determination of tumor fraction or diagnosis of cancer. Furthermore, the probes can be designed to target genomic regions that are determined to have an abnormal methylation pattern and/or hypermethylation or hypomethylation patterns to provide additional selectivity and specificity of the detection.

In some embodiments, a probe (or probe pair) in the plurality of probes targets genomic regions comprising at least 25 bp, 30 bp, 35 bp, 40 bp, 45 bp, 50 bp, 60 bp, 70 bp, 80 bp, or 90 bp. In some embodiments, a probe in the plurality of probes targets genomic regions containing at least 5 methylation sites. In some embodiments, a probe in the plurality of probes targets genomic regions containing less than 20, 15, 10, 8, or 6 methylation sites. In some embodiments, a probe in the plurality of probes targets genomic regions having at least 80, 85, 90, 92, 95, or 98% of methylation (e.g., CpG) sites that are either methylated or unmethylated in non-cancerous or cancerous samples.

Filtering Cell-Free Fragments.

In some embodiments, the method further comprises applying one or more filter conditions to the plurality of cell-free fragments. Thus, in some embodiments, not all cell-free fragments obtained from a methylation sequencing of the one or more nucleic acid samples are used to identify a plurality of features for estimating subject cell source fractions and/or used to estimate subject cell source fractions. In some embodiments, this is due to the fact that nucleic acid fragments (e.g., cell-free nucleic acids) vary in terms of information content, and in some embodiments only those nucleic acid fragments with the desired information content are retained for feature identification and/or cell source fraction estimation (e.g., fragments that do not provide relevant information are discarded). In some embodiments, features are determined from cell-free fragments that satisfy one or more filter conditions in a plurality of filtering conditions (e.g., where each filter condition evaluates the information content of the fragments). Multiple filtering methods are described, for example, in detail in International Patent Application No. PCT/US2020/034317, entitled “Systems and Methods for Determining Whether a Subject has a Cancer Condition Using Transfer Learning,” filed May 22, 2020, and in U.S. patent application Ser. No. 16/352,602, entitled “Anomalous fragment detection and classification,” filed Mar. 13, 2019, now published as US2019/0287652, each of which is hereby incorporated by reference. Non-limiting examples of filter conditions are provided below.

P-Value Filtering Based on Methylation Vectors.

In some embodiments, a filter condition in the plurality of filter conditions is a requirement that each cell-free fragment in the plurality of cell-free fragments have a corresponding p-value that is below a threshold value, where the p-value is determined by p-value filtering as described Example 5 in International Patent Application No. PCT/US2020/034317, entitled “Systems and Methods for Determining Whether a Subject has a Cancer Condition Using Transfer Learning,” filed May 22, 2020, and in U.S. patent application Ser. No. 16/352,602, entitled “Anomalous fragment detection and classification,” filed Mar. 13, 2019, now published as US2019/0287652, each of which is hereby incorporated herein by reference in its entirety. The goal of such a filter condition is to accept and use anomalously methylated cell-free fragments based on their corresponding methylation state vectors. For example, for each cell-free fragment in a sample, a determination is made as to whether the fragment is anomalously methylated (e.g., via analysis of sequence reads derived therefrom), relative to an expected methylation state vector using the methylation state vector corresponding to the fragment (e.g., where the expected methylation state vector is determined from sequence analysis of a cohort (plurality) of healthy subjects). The generation of methylation state vectors for such cell-free fragments is disclosed, for example, in U.S. Pat. Appl. Pub. No. 2019/0287652, which is hereby incorporated herein by reference in its entirety.

In some embodiments, the healthy cohort comprises at least twenty subjects and the plurality of cell-free fragments comprises at least 10,000 different corresponding methylation patterns. In some embodiments, the healthy cohort comprises at least 10, at least 20, at least 30, at least 40, at least 50, at least 60, at least 70, at least 80, at least 90, or at least 100 subjects. In some embodiments, the healthy cohort comprises between 1 and 10, between 10 and 50, between 50 and 100, between 100 and 500, between 500 and 1000, or more than 1000 subjects. In some embodiments, the plurality of cell-free fragments comprises between 1 and 1000, between 1000 and 2000, between 2000 and 4000, between 4000 and 6000, between 6000 and 8000, between 8000 and 10,000, between 10,000 and 20,000, between 20,000 and 50,000, or more than 50,000 different corresponding methylation patterns.

In some embodiments, the p-value threshold is between 0.001 and 0.20. In some embodiments, the threshold value is 0.01 (e.g., p must be <0.01 in such embodiments). In some embodiments, the threshold value is 0.001, 0,005, 0.01, 0.015, 0.02, 0.05, or 0.10. In some embodiments, the threshold value is between 0.0001 and 0.20. In some embodiments, the p-value threshold is satisfied for a methylation pattern from the subject when the corresponding methylation pattern for each respective cell-free fragment in the plurality of cell-free fragments has a p-value of 0.10 or less, 0.05 or less, or 0.01 or less.

In such embodiments, only those cell-free fragments that have a p-value below the threshold value contribute to feature identification and/or cell source fraction estimation. For example, in some embodiments, the plurality of cell-free fragments is filtered by removing from the plurality of cell-free fragments each respective cell-free fragment whose corresponding methylation pattern (e.g., methylation state vector) across a corresponding plurality of CpG sites in the respective fragment has a p-value that fails to satisfy a p-value threshold.

In some embodiments, anomalous fragments are identified as fragments with over a threshold number of CpG sites and either with over a threshold percentage of the CpG sites methylated (hypermethylated) or with over a threshold percentage of CpG sites unmethylated (hypomethylated). See, for example, the filter conditions based on minimum CpG sites and/or fragment length described below. In some embodiments, the threshold percentage of methylated and/or unmethylated CpG sites is at least 50%, at least 60%, at least 70%, at least 80%, at least 85%, at least 90%, or at least 95%. In some embodiments, the threshold percentage of methylated and/or unmethylated CpG sites is between 50% and 100%.

In some embodiments, a Markov model (e.g., a Hidden Markov Model “HMM”) is used to determine the probability that a sequence of methylation states (comprising, e.g., “M” for methylated and/or “U” for unmethylated) will be observed for each respective cell-free fragment, given a set of probabilities that determine, for each state in the methylation pattern of the respective fragment, the likelihood of observing the next state in the sequence. In some embodiments, the set of probabilities are obtained by training the HMM. Such training involves computing statistical parameters (e.g., the probability that a first state will transition to a second state (the transition probability) and/or the probability that a given methylation state will be observed for a respective CpG site (the emission probability)), given an initial training dataset of observed methylation state sequences (e.g., methylation patterns) obtained from a cohort of non-cancer subjects. In some embodiments, the HMI is trained using supervised training (e.g., using samples where the underlying sequence as well as the observed states are known). In some alternative embodiments, the HMI is trained using unsupervised training (e.g., Viterbi learning, maximum likelihood estimation, expectation-maximization training, and/or Baum-Welch training). For example, an expectation-maximization algorithm such as the Baum-Welch algorithm estimates the transition and emission probabilities from observed sample sequences and generates a parameterized probabilistic model that best explains the observed sequences. Such algorithms iterate the computation of a likelihood function until the expected number of correctly predicted states is maximized. See, e.g., Yoon, 2009, “Hidden Markov Models and their Applications in Biological Sequence Analysis,” Curr. Genomics. September; 10(6): 402-415, doi: 10.2174/138920209789177575.

Minimum Bag-Size.

In some embodiments, a filter condition in the plurality of filter conditions is a requirement that each cell-free fragment have a bag-size greater than a threshold integer. In other words, in some embodiments, a filter condition in the one or more filter conditions is application of a requirement that each respective cell-free fragment in the plurality of cell-free fragments is represented by a threshold number of sequence reads in a corresponding plurality of sequence reads measured from the one or more nucleic acid samples comprising the respective fragment in the corresponding biological sample. For example, in the case where the threshold integer is one, the filter condition is application of a requirement that each cell-free fragment be represented by more than one sequence read in the corresponding plurality of sequence reads measured from the biological sample. In some embodiments, the threshold integer is 1, 2, 3, 4, 5, 6, 7, 8, 9, 10, or an integer between 10 and 100. In some embodiments, the threshold integer is between 1 and 10, between 10 and 20, between 20 and 30, between 30 and 40, between 40 and 50, between 50 and 60, between 60 and 70, between 70 and 80, between 80 and 90, or between 90 and 100. In some embodiments, the threshold integer is between 100 and 500, between 500 and 1000, or more than 1000.

In some embodiments, a filter condition in the plurality of filter conditions is a requirement that each cell-free fragment have a bag-size greater than a threshold integer, where the sequence reads in each respective bag (e.g., representing the respective cell-free fragment) is obtained from a sequencing of a plurality of cell-free nucleic acids. For example, in some embodiments, a filter condition in the one or more filter conditions is application of a requirement that each respective cell-free fragment in the plurality of cell-free fragments is represented by a threshold number of cell-free nucleic acids in the one or more nucleic acid samples comprising the respective fragment in the corresponding biological sample. In some embodiments, the threshold integer is 1, 2, 3, 4, 5, 6, 7, 8, 9, 10, or an integer between 10 and 100. In some embodiments, the threshold integer is between 1 and 10, between 10 and 20, between 20 and 30, between 30 and 40, between 40 and 50, between 50 and 60, between 60 and 70, between 70 and 80, between 80 and 90, or between 90 and 100. In some embodiments, the threshold integer is between 100 and 500, between 500 and 1000, or more than 1000.

Minimum Number of CpG Sites.

In some embodiments, a filter condition in the one or more filter conditions is application of a requirement that each respective cell-free fragment in the plurality of cell-free fragments have a threshold number of CpG sites. In some embodiments, the threshold number of CpG sites is at least 1, 2, 3, 4, 5, 6, 7, 8, 9 or 10 CpG sites. In some embodiments, the threshold number of CpG sites is between 1 and 10, between 10 and 20, between 20 and 30, between 30 and 40, between 40 and 50, or more than 50 CpG sites.

In some embodiments, a filter condition in the one or more filter conditions is a requirement that each respective cell-free fragment in the plurality of cell-free fragments have a length of less than a threshold number of base pairs. In some embodiments, the threshold number of base pairs is one thousand, two thousand, three thousand, or four thousand base pairs. In some embodiments, the threshold number of base pairs is 100, 200, 300, 400, 500, 600, 700, 800, 900, or 1000 base pairs. In some embodiments, the threshold number of base pairs is one thousand, two thousand, three thousand, or four thousand contiguous base pairs in length. In some embodiments, the threshold number of base pairs is 100, 200, 300, 400, 500, 600, 700, 800, 900, or 1000 contiguous base pairs in length.

In some embodiments, a filter condition in the plurality of filter conditions is a requirement that each cell-free fragment covers a first threshold number of CpG sites and be less than a second threshold length in terms of base pairs. For example, in the case where the first threshold is 1 CpG site and the second threshold 1000 base pairs, each cell-free fragment must cover more than one CpG site and be less than 1000 base pairs in length. In some embodiments, each cell-free fragment must cover at least 1, 2, 3, 4, 5, 6, 7, 8, 9, 10, 11, 12, 13, 14, 15, 16, 17, 18, 19, or 20 CpG sites within a particular fragment length (e.g., the second threshold length). In some embodiments, each cell-free fragment must be less than 500, 1000, 2000, 3000, or 4000 contiguous base pairs in length while spanning a particular number of CpG sites (e.g., the first threshold number). In other words for example, in some embodiments, the filter condition in the plurality of filter conditions requires that each cell-free fragment include at least 1 CpG site, at least 2 CpG sites, at least 3 CpG sites, at least 4 CpG sites, at least 5 CpG sites, at least 6 CpG sites, at least 7 CpG sites, at least 8 CpG sites, at least 9 CpG sites, at least 10 CpG sites, at least 11 CpG sites, at least 12 CpG sites, at least 13 CpG sites, at least 14 CpG sites, or at least 15 CpG sites within less than 500 contiguous nucleotides of the reference genome.

Hypermethylation or Hypomethylation.

In some embodiments, a filter condition in the plurality of filter conditions is a requirement that each cell-free fragment is hypermethylated. In some embodiments, a filter condition in the plurality of filter conditions is a requirement that each cell-free fragment is hypomethylated. In some embodiments, the filter condition is dependent on a region of a genome (e.g., a bin). For instance, a number of regions of the human genome having a hypermethylated state that is associated with one or more cancer conditions, as well as a number of regions of the human genome having a hypomethylated state that is associated with one or more cancer conditions, are disclosed in International Patent Application No. PCT/US2019/025358, published as WO2019/195268A2, entitled “Methylated Markers and Targeted Methylation Probe Panels,” filed Apr. 2, 2019, International Patent Application No. PCT/US2020/015082, published as WO2020/154682A2, entitled “Detecting Cancer, Cancer Tissue or Origin, or Cancer Type,” filed Jan. 24, 2020, and International Patent Application No. PCT/US2019/053509, published as WO2020/069350A1, entitled “Methylated Markers and Targeted Methylation Probe Panel,” filed Sep. 27, 2019, each of which is hereby incorporated by reference herein in its entirety. Accordingly, in some embodiments of the present disclosure, one or more bins in a plurality of genomic regions each represent a corresponding genomic region in the regions disclosed in International Patent Publication Nos. WO2019/195268, WO2020/154682, and/or WO2020/069350, and a filter condition in the plurality of filter conditions (a) requires selection of cell-free fragments that are hypermethylated when selecting cell-free fragments that map to a bin representing a region of the human genome that has a hypermethylated state that is associated with one or more cancer conditions of CpG sites as indicated by International Patent Publication Nos. WO2019/195268, WO2020/154682, and/or WO2020/069350 and (b) requires selection of cell-free nucleic acids that are hypomethylated when selecting fragments that map to a bin representing a region of the human genome that has a hypomethylated state that is associated with one or more cancer conditions of CpG sites as indicated by International Patent Publication Nos. WO2019/195268, WO2020/154682, and/or WO2020/069350.

In some embodiments, the plurality of filter conditions requires that the p-value threshold is satisfied and that the cell-free fragment is hypermethylated. In some embodiments, the plurality of filter conditions requires that the p-value threshold is satisfied and that the cell-free fragment is hypomethylated. In some embodiments, the plurality of filter conditions is different for each bin. For instance, for one bin in the plurality of bins, the plurality of filter conditions requires that the p-value threshold is satisfied and that the cell-free fragment is hypomethylated, while for a second bin in the plurality of bins, the plurality of filter conditions requires that the p-value threshold is satisfied and that the cell-free fragment is hypermethylated.

Cancer Condition.

In some embodiments, a filter condition in the plurality of filter conditions is a requirement that each cell-free fragment satisfy a cancer condition threshold (e.g., that each cell-free fragment have a probability above a predefined threshold of being associated with a respective cancer condition). In some embodiments, each cancer condition has a different respective predefined threshold. For example, as described in U.S. Patent Application No. 63/003,087, entitled Systems and Methods for Using Neural Networks to Determine a Cancer State, filed on Mar. 31, 2020, which is hereby incorporated by reference in its entirety, a trained neural network (e.g., trained on a plurality of reference subjects) is used to determine cancer probabilities for each genomic region (e.g., bin).

In some such embodiments, for each respective bin in the plurality of bins, for each respective cell-free fragment in the plurality of cell-free fragment that map to the respective bin, a corresponding trained neural network computes a prediction value that is the probability that the cell-free fragment is associated with a cancer condition (e.g., a presence of cancer) based on the methylation pattern of the respective cell-free fragment. Thus, in some such embodiments, the methylation pattern of the respective cell-free fragment is scored using the trained neural network, where the score outputted by the trained neural network comprises the probability that the cell-free fragment has the cancer condition and/or a calculation based on the probability that the cell-free fragment is associated with the cancer condition (e.g., a presence of cancer). The respective cell-free fragment passes the filter condition (e.g., is selected for use in identifying features for estimating cell source fraction, and/or is selected for use in estimating cell source fraction) if the resulting score satisfies the condition defined above (e.g., a probability that is above a fixed value threshold). The respective cell-free fragment does not pass the filter condition (e.g., is discarded) if the resulting score does not satisfy the condition defined above (e.g., a probability that is below a fixed value threshold).

In some such embodiments, the threshold value is positive or negative. In some embodiments, the threshold value is between 0.1 and 1, between 1 and 5, between 5 and 10, between 10 and 50, between 50 and 100, or greater than 100. In some embodiments, the threshold value is between −0.1 and −1, between −1 and −5, between −5 and −10, between −10 and −50, between −50 and −100, or less than −100. In some embodiments, the threshold value is zero. In some embodiments, each bin has a respective threshold for each respective cancer condition (e.g., a respective subset of bins is associated with each cancer condition).

In some embodiments, any combination of the disclosed filter conditions is imposed. In some embodiments, the plurality of cell-free fragments comprises one or more cell-free fragments whose methylation patterns satisfy one or more filter conditions disclosed herein.

Mapping Fragments and Bins.

Block 210. In Block 210, the method proceeds by mapping each cell-free fragment in each plurality of cell-free fragments to a bin in a plurality of bins, and thereby obtaining a plurality of training sets of cell-free fragments. Each respective bin in the plurality of bins represents a corresponding portion of a human reference genome. Each training set of cell-free fragments is mapped to a different bin in the plurality of bins.

In some embodiments, mapping is performed using a Smith-Waterman gapped alignment as implemented in, for example Arioc, or a Burrows-Wheeler transform as implemented in, for example Bowtie. Other suitable alignment programs include, but are not limited to BarraCUDA, BBMap, BFAST, BigBWA, BLASTN, BLAT, BWA, BWA-PSSM, CASHX. See, for example, Langmead and Salzberg, 2012, Nat Methods 9, pp. 357-359; Li and Durbin, 2009, “Fast and accurate short read alignment with Burrows-Wheeler transform,” Bioinformatics 25(14), 1754-1760; and Smith and Yun, 2017, “Evaluating alignment and variant-calling software for mutation identification in C. elegans by whole-genome sequencing,” PLOS ONE, doi.org/10.1371/journal.pone.0174446, each of which is hereby incorporated by reference. In some embodiments, mapping each cell-free fragment to a bin in the plurality of bins allows mismatching. In some embodiments, the mapping comprises at least 1, at least 2, at least 3, at least 4, at least 5, at least 6, at least 7, at least 8, at least 9, at least 10, or more than 10 mismatches.

In some embodiments, referring to Block 212, the plurality of bins consists of or comprises between 1000 and 100,000 bins. In some embodiments, the plurality of bins consists of or comprises between 15,000 and 80,000 bins. In some embodiments, the plurality of bins consists of or comprises between 25,000 and 65,000 bins. In some embodiments, the plurality of bins consists of or comprises between 45,000 and 65,000 bins.

In some embodiments, the plurality of bins comprises at least 1000 bins, at least 2500 bins, at least 5000 bins, at least 10,000 bins, at least 20,000 bins, at least 30,000 bins, at least 40,000 bins, at least 50,000 bins, at least 60,000 bins, at least 70,000 bins, at least 80,000 bins, at least 90,000 bins, at least 100,000 bins, or at least 110,000 bins.

Further, in some embodiments, in accordance with Block 214 of FIG. 2A, each respective bin in the plurality of bins has, on average, between 10 and 1200 residues (e.g., each bin corresponds to a portion of a human reference genome that consists of between 10 and 1200 nucleotides). In some embodiments, each respective bin in the plurality of bins has, on average, between 10 and 10,000 residues. In some embodiments, each respective bin in the plurality of bins has, on average, between 10 and 500 residues. In some embodiments, each respective bin in the plurality of bins has, on average, between 10 and 100 residues. In some embodiments, each respective bin in the plurality of bins has, on average, between 25 and 100 residues. In some embodiments, each respective bin in the plurality of bins has, on average, between 5000 and 10,000 residues.

In some embodiments, each respective bin in the plurality of bins comprises less than 10 residues, less than 20 residues, less than 30 residues, less than 40 residues, less than 50 residues, less than 60 residues, less than 70 residues, less than 80 residues, less than 90 residues, less than 100 residues, less than 200 residues, less than 300 residues, less than 400 residues, less than 500 residues, less than 600 residues, less than 700 residues, less than 800 residues, less than 900 residues, less than 1000 residues, less than 2000 residues, less than 3000 residues, less than 4000 residues, less than 5000 residues, less than 6000 residues, less than 7000 residues, less than 8000 residues, or less than 9000 residues.

Referring to Block 216, in some embodiments, each bin in the plurality of bins comprises 2, 3, 4, 5, 6, 7, 8, 9, 10, 11, 12, 13, 14, 15, 16, 17, 18, 19, 20 or more CpG sites. In some embodiments, each bin in the plurality of bins comprises 2, 3, 4, 5, 6, 7, 8, 9, 10, 11, 12, 13, 14, 15, 16, 17, 18, 19, 20 or more contiguous CpG sites. In some embodiments, each bin in the plurality of bins consists of between 2 and 100 contiguous CpG sites in a human reference genome. In some embodiments, each bin in the plurality of bins consist of between 2 and 50 contiguous CpG sites. In some embodiments, each bin in the plurality of bins consists of between 50 and 100 contiguous CpG sites. In some embodiments, each bin in the plurality of bins consists of at least 2 contiguous CpG sites.

In some embodiments, the plurality of bins is constructed by dividing all or a portion of a reference genome (e.g., mammalian, human, etc.) into equally sized bins, where each bin represents a unique equally sized part of the reference genome. In some embodiments, the plurality of bins is constructed by dividing all or a portion of a reference genome (e.g., mammalian, human, etc.) into equally or unequally sized bins, where each bin represents a unique part of the reference genome.

In some embodiments, the plurality of bins is constructed by dividing all or a portion of a reference genome (e.g., mammalian, human, etc.) into equally or unequally sized bins, where each bin represents a corresponding part of the reference genome. In such embodiments, the corresponding part of the reference genome represented by one bin in the plurality of bins can overlap with the corresponding part of the reference genome represented by another bin in the plurality of bins. In some such embodiments, the plurality of bins is constructed by dividing all of a reference genome (e.g., mammalian, human, etc.) into equally or unequally sized bins, where each bin represents a corresponding overlapping or non-overlapping part of the reference genome. In some embodiments, the plurality of bins is constructed by dividing a portion of a reference genome (e.g., mammalian, human, etc.) into equally or unequally sized bins, where each bin represents an overlapping or non-overlapping part of the reference genome.

In some embodiments, the plurality of bins is constructed such that at least some of the regions of the human genome implicated in absence or presence of cancer are represented by the plurality of bins whereas other regions of the reference genome are not represented by the bins. Regardless of approach, each bin represents a unique part of the reference genome. In some embodiments, such bins range in size between 30 bps and 5000 bps, between 30 bps and 4000 bps, between 30 bps and 3000 bps, between 30 bps and 2000 bps, between 30 bps and 1000 bps, or between 40 bps and 800 bps of the reference genome. In alternative embodiments, such bins range in size between 10,000 bps and 100,000 bps, between 20,000 bps and 300,000 bps, between 30,000 bps and 500,000 bps, between 40,000 bps and 1,000,000 bps between 50,000 bps and 5,000,000 bps, or between 100,000 bps and 25,000,000 bps of the reference genome.

In some embodiments, the portion of the reference genome is between 1 and 22 chromosomes of the reference genome, or at least 25 percent, at least 30 percent, at least 35 percent, at least 40 percent, at least 45 percent, at least 50 percent, at least 55 percent, at least 60 percent, at least 65 percent, at least 70 percent, at least 75 percent, at least 80 percent, at least 85 percent, at least 90 percent, at least 95 percent, or at least 99 percent of the reference genome. In some such embodiments, each bin represents between 10,000 bases and 100,000 bases, between 20,000 bases and 300,000 bases, between 30,000 bases and 500,000 bases, between 40,000 bases and 1,000,000 bases between 50,000 bases and 5,000,000 bases, or between 100,000 bases and 25,000,000 bases of the reference genome.

In some embodiments, each of the bins represents a specific site of a reference genome that has been identified as being associated with cancer.

In some embodiments, each of the bins represents a specific region of a reference genome that has been identified as being associated with cancer through cancer- and/or tissue-specific methylation patterns in cfDNA relative to non-cancer controls.

In some embodiments, each bin represents all or a portion of an enhancer, promoter, 5′ UTR, exon, exon/inhibitor boundary, intron, intron/exon boundary, 3′ UTR region, CpG shelf, CpG shore, or CpG island in a reference genome. See, for example, Cavalcante and Santor, 2017, “annotatr: genomic regions in context,” Bioinformatics 33(15) 2381-2383, for suitable definitions of such regions and where such annotations are documented for a number of different species.

In some embodiments, genomic regions with high variability or low mappability are excluded from bin representation in the plurality of bins, for example, using the methods disclosed in Jensen et al, 2013, PLoS One 8; e57381. See also, Li and Freudenberg, 2014, Front. Genet. 5, p. 318, for analysis of mappability.

Select Human Genomic Regions Used for Bins.

In some embodiments of the present disclosure, each bin in the plurality of bins is drawn from a panel of genomic regions that is designed for targeted selection of cancer-specific methylation patterns. In some embodiments, each such genomic region is drawn from Table 2 of International Patent Application No. PCT/US2020/015082, published as WO2020/154682A2, entitled “Detecting Cancer, Cancer Tissue or Origin, or Cancer Type,” filed Jan. 24, 2020, which is hereby incorporated by reference, including the Sequence Listing referenced therein. SEQ ID NOs 452,706-483,478 of PCT/US2020/015082 provide further information about certain hypermethylated or hypomethylated target genomic regions. These SEQ ID NO records identify target genomic regions that can be differentially methylated in samples from specified pairs of cancer types. The target genomic regions of SEQ ID NOs 452,706-483,478 of PCT/US2020/015082 are drawn from list 6 of PCT/US2020/015082. Many of the same target genomic regions are also found in lists 1-5 and 7-16 of PCT/US2020/015082. The entry for each SEQ ID indicates the chromosomal location of the target genomic region relative to hg19, whether cfDNA fragments to be enriched from the region are hypermethylated or hypomethylated, the sequence of one DNA strand of the target genomic region, and the pair or pairs of cancer types that are differentially methylated in that genomic region. As the methylation status of some target genomic regions distinguish more than one pair of cancer types, each entry identifies a first cancer type as indicated in Table 3 of PCT/US2020/015082, including the Sequence Listing referenced therein and one or more second cancer types.

In some embodiments, the plurality of bins of the present disclosure includes a separate bin for each of at least 200, 500, 1,000, 5,000, 10,000, 15,000, 20,000, 30,000, 40,000, or 50,000 target genomic regions in any one of lists 1-16, lists 1-3, lists 13-16, list 12, list 4, or lists 8-11 of PCT/US2020/015082. In some embodiments, the plurality of bins of the present disclosure includes a separate bin for each of at least 200, 500, 1,000, 5,000, 10,000, 15,000, 20,000, 30,000, 40,000, or 50,000 target genomic regions in any combination of one or more lists 1-16 of PCT/US2020/015082 (e.g., such as lists 1-3, lists 13-16, list 12, list 4, or lists 8-11).

In some embodiments, the plurality of bins of the present disclosure includes a separate bin for each of at least 20%, 30%, 40%, 50%, 60%, 70%, 80%, 90%, or 95% of the target genomic regions in any one of lists 1-16 of PCT/US2020/015082. In some embodiments, the plurality of bins of the present disclosure includes a separate bin for each of at least 20%, 30%, 40%, 50%, 60%, 70%, 80%, 90% or 95% of the target genomic regions in any combination of one or more lists 1-16 of PCT/US2020/015082 (e.g., such as lists 1-3, lists 13-16, list 12, list 4, or lists 8-11).

Additional Select Human Genomic Regions Used for Bins.

In some embodiments of the present disclosure, each bin in the plurality of bins is drawn from a panel of genomic regions that is designed for targeted selection of cancer-specific methylation patterns. In some embodiments, each such genomic region is drawn from Table 2 of International Patent Application No. PCT/US2019/053509, published as WO2020/069350A1, entitled “Methylated Markers and Targeted Methylation Probe Panel,” filed Sep. 27, 2019, which is hereby incorporated by reference, including the Sequence Listing referenced therein.

The sequence listing of WO2020/069350A1 includes the following information: (1) SEQ ID NO, (2) a sequence identifier that identifies (a) a chromosome or contig on which the CpG site is located and (b) a start and stop position of the region, (3) the sequence corresponding to (2) and (4) whether the region was included based on its hypermethylation or hypomethylation score. The chromosome numbers and the start and stop positions are provided relative to a known human reference genome, GRCh37/hg19. The sequence of GRCh37/hg19 is available from the National Center for Biotechnology Information (NCBI), the Genome Reference Consortium, and the Genome Browser provided by Santa Cruz Genomics Institute.

Generally, a bin can encompass any of the CpG sites included within the start/stop ranges of any of the targeted regions included in lists 1-8 of WO2020/069350.

In some embodiments, the plurality of bins of the present disclosure includes a separate bin for each of at least 200, 500, 1,000, 5,000, 10,000, 15,000, 20,000, 30,000, 40,000, or 50,000 target genomic regions in any one of lists 1-8 of WO2020/069350. In some embodiments, the plurality of bins of the present disclosure includes a separate bin for each of at least 200, 500, 1,000, 5,000, 10,000, 15,000, 20,000, 30,000, 40,000, or 50,000 target genomic regions in any combination of lists 1-8 of WO2020/069350.

In some embodiments, the plurality of bins of the present disclosure includes a separate bin for each of at least 20%, 30%, 40%, 50%, 60%, 70%, 80%, 90%, or 95% of the target genomic regions in any one of lists 1-8 of WO2020/069350. In some embodiments, the plurality of bins of the present disclosure includes a separate bin for each of at least 20%, 30%, 40%, 50%, 60%, 70%, 80%, 90% or 95% of the target genomic regions in any combination of lists 1-8 of WO2020/069350.

In some embodiments of the present disclosure, each bin in the plurality of bins is drawn from a panel of genomic regions that is designed for targeted selection of cancer-specific methylation patterns. In some embodiments, each such bin corresponds to a genomic region in any of Table 1-24 of International Patent Application No. PCT/US2019/025358, published as WO2019/195268A2, entitled “Methylated Markers and Targeted Methylation Probe Panels,” filed Apr. 2, 2019, which is hereby incorporated herein by reference in its entirety.

In some embodiments, each bin of the present disclosure maps to a genomic region listed in one or more of Table 1, 2, 3, 4, 5, 6, 7, 8, 9, 10, 11, 12, 13, 14, 15, 16, 17, 18, 19, 20, 21, 22, 23 and/or 24 of WO2019/195268A2.

In some embodiments, an entirety of plurality of the bins of the present disclosure together are configured to map to at least 30%, 40%, 50%, 60%, 70%, 80%, 90% or 95% of the genomic regions in one or more of Tables 1-24 of WO2019/195268A2. In some such embodiments, each bin in the plurality of bins maps to a single unique corresponding genomic region in any of Tables 1-24 of WO2019/195268A2. In some such embodiments, a bin in the plurality of bins of the present disclosure map to one, two, three, four, five, six, seven, eight, nine or ten unique corresponding genomic regions in any combination of Tables 1-24 of WO2019/195268A2.

In some such embodiments, each bin in the plurality of bins of the present disclosure maps to a single unique corresponding genomic region in any of Tables 2-10 or 16-24 of WO2019/195268A2. In some such embodiments, a bin in the plurality of bins maps to one, two, three, four, five, six, seven, eight, nine or ten unique corresponding genomic region in any combination of Tables 2-10 or 16-24 of WO2019/195268A2.

In some embodiments, one or more bins in the plurality of bins of the present disclosure together are configured to map to at least 30%, 40%, 50%, 60%, 70%, 80%, 90% or 95% of the genomic regions in Tables 1, 2, 3, 4, 5, 6, 7, 8, 9, 10, 11, 12, 13, 14, 15, 16, 17, 18, 19, 20, 21, 22, 23, and/or 24 of WO2019/195268A2.

Assigning Cell-Free Fragment Cancer Conditions.

Block 218. Referring to Block 218 of FIG. 2B, the method proceeds by assigning a cell-free fragment cancer condition to each respective cell-free fragment in each training set of cell-free fragments in the plurality of training sets of cell-free fragments, where the cell-free fragment cancer condition is one of the first cancer condition and the second cancer condition, as a function of an output of a classifier upon inputting a methylation pattern of the respective cell-free fragment into the classifier.

In some embodiments, the classifier has the form:

$$R ( fragment ) ≡ log ( \frac{ℙ ( fragment \vert first cancer condition )}{ℙ ( fragment \vert second cancer condition )} )$$

In some such embodiments, [symbol] (fragment|first cancer condition class) is a first model for the first cancer condition.

In some such embodiments, [symbol] (fragment|second cancer condition class) is a second model for the second cancer condition. In some embodiments, with regards to the first and second models, “fragment” refers to the methylation pattern of the respective cell-free fragment. In some embodiments, the cell-free fragment cancer condition of the respective fragment is assigned the first cancer condition when R(fragment) satisfies a threshold value. In some embodiments, the threshold values is any value between 1 and 10. In some embodiments, the threshold value is 1, 2, 3, 4, 5, 6, 7, 8, 9, or 10.

In some embodiments, the first model is a first mixture model comprising a first plurality of sub-models, the second model is a second mixture model comprising a second plurality of sub-models, and each sub-model in the first and second plurality of sub-models represents an independent corresponding methylation model for a source of cell-free fragments in the corresponding biological sample.

In some embodiments, the subject cancer condition is one of a plurality of cancer conditions (e.g., where the plurality of cancer conditions comprises N cancer conditions). In some such embodiments, the classifier has the form:

$$R ( fragment ) ≡ log ( \frac{ℙ ( fragment \vert 1 st cancer condition )}{\begin{matrix}\begin{matrix}ℙ ( fragment \vert 2 nd cancer condition ) + \\ ℙ ( fragment \vert 3 rd cancer condition ) + … +\end{matrix} \\ ℙ ( fragment \vert {N}^{th} cancer condition )\end{matrix}} )$$

In some such embodiments, [symbol] (fragment|3rd cancer condition) is a third model for a third cancer condition in the plurality of cancer conditions. In some embodiments, [symbol] (fragment|N^{th} cancer condition) is an N^{th} model for the N^{th} cancer condition in the plurality of cancer conditions.

Examples of mixtures models for use in accordance with embodiments herein are described in U.S. Patent Application No. 62/847,223, entitled “Model-Based Featurization and Classification” filed May 13, 2019, which is hereby incorporated in its entirety by reference.

In some embodiments, each independent corresponding methylation model is one of a binomial model, beta-binomial model, independent sites model or Markov model. In some embodiments, two or more sub-models in the first plurality of sub-models are independent sites models, and two or more sub-models in the second plurality of sub-models are independent sites models.

For example, U.S. Patent Application No. 62/983,443, entitled “Identifying Methylation Patterns that Discriminate or Indicate a Cancer Condition,” filed on Feb. 28, 2020, which is hereby incorporated by reference in its entirety, discloses multiple methods of identifying methylation patterns that discriminate specific cancer conditions of the subject. Specifically, in some embodiments, each cancer condition (e.g., cancer of origin) in the group of cancer conditions corresponds to a respective pattern of abnormal methylation (e.g., a qualifying methylation pattern) across a reference genome or across a subset of the reference genome (e.g., as evaluated by targeted panel sequencing). To determine the cancer condition of a particular subject, the method evaluates a plurality of genomic regions of interest, and generates, for each genomic region in the plurality of genomic regions, a corresponding count of fragments with methylation patterns that map to the respective genomic region (e.g., there is a respective count of fragments for each possible methylation pattern identified in fragments mapping to the respective genomic region). The method then compares the fragment counts across the plurality of genomic regions for the subject to a database (e.g., library) of methylation patterns corresponding to different cancer conditions (e.g., where each cancer condition has corresponding fragment counts for a respective subset of genomic regions within the plurality of genomic regions) to determine a probable cancer condition for the subject, where the cancer condition corresponds to cancer vs. non-cancer, type of cancer, and/or tissue-of-origin. In some embodiments, the method is used to identify a cancer condition of the subject for input into downstream applications (e.g., for estimating tumor fraction and/or determining minimal residual disease of the subject). In some embodiments, the plurality of bins used in the present disclosure are selected to represent portions of the genome identified in U.S. Patent Application No. 62/983,443 that contain the methylation patterns associated with any single or any combination of cancers evaluated in U.S. Patent Application No. 62/983,443.

As another example, U.S. patent application Ser. No. 15/931,022, entitled “Model-Based Featurization and Classification,” filed on May 13, 2020, which is hereby incorporated by reference in its entirety, discloses the development of probabilistic models using methylation states of genomic regions (e.g., determined from fragments as represented by sequence reads that map to the genomic regions) to identify methylation features that correspond to distinct cancer conditions. In some embodiments, the plurality of bins used in the present disclosure are selected to represent portions of the genome identified in U.S. patent application Ser. No. 15/931,022 that contain the methylation patterns associated with any single or any combination of cancers evaluated in U.S. patent application Ser. No. 15/931,022.

Other methods for performing cancer classification on nucleic acid fragments include those disclosed in, for example, U.S. Patent Application No. 62/948,129, entitled “Cancer Classification using Patch Convolutional Neural Networks,” filed Dec. 13, 2019, U.S. patent application Ser. No. 16/352,739, entitled “Method and System for Selecting, Managing, and Analyzing Data of High Dimensionality,” filed Mar. 13, 2019, U.S. patent application Ser. No. 16/428,575, entitled “Convolutional Neural Network Systems and Methods for Data Classification,” filed May 31, 2019, and U.S. Patent Application No. 62/985,258, entitled “Systems and Methods for Cancer Condition Determination using Autoencoders,” filed Mar. 4, 2020, each of which is hereby incorporated herein by reference in its entirety.

In some embodiments, the classifier is a multivariate logistic regression, a neural network, a convolutional neural network, a support vector machine (SVM), a decision tree, a regression algorithm, or a supervised clustering model.

Logistic regression algorithms, including multivariate logistic regression, are disclosed in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, are disclosed in See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

SVM algorithms are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training set (e.g., by tumor fraction value) with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973.

Once a method for measuring “similarity” or “dissimilarity” between points in a dataset has been selected, clustering requires a criterion function that measures the clustering quality of any partition of the data. Partitions of the dataset that extremize the criterion function are used to cluster the data. See page 217 of Duda 1973. Criterion functions are discussed in Section 6.8 of Duda 1973.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. Such clustering can be on the set of first features {p₁, . . . , p_{N-K}} (or the principal components derived from the set of first features). In some embodiments, the clustering comprises unsupervised clustering where no preconceived notion of what clusters should form when the training set is clustered are imposed.

Identifying Features.

Block 220. Referring to Block 220 of FIG. 2B, the method proceeds by determining, for each respective bin in the plurality of bins, a corresponding measure of association I between (a) the subject cancer condition of respective training subjects in the plurality of training subjects and (b) the cell-free fragment cancer condition of respective cell-free fragments in the corresponding training set of cell-free fragments mapping to the respective bin.

In some embodiments, with regard to Block 222, the measure of association is a correlation. Referring to Block 224, in some embodiments, the correlation is a Pearson correlation coefficient. Referring to Block 226, in some embodiments, the correlation is performed using an adjusted correlation coefficient, weighted correlation, reflective correlation coefficient, or scaled correlation coefficient.

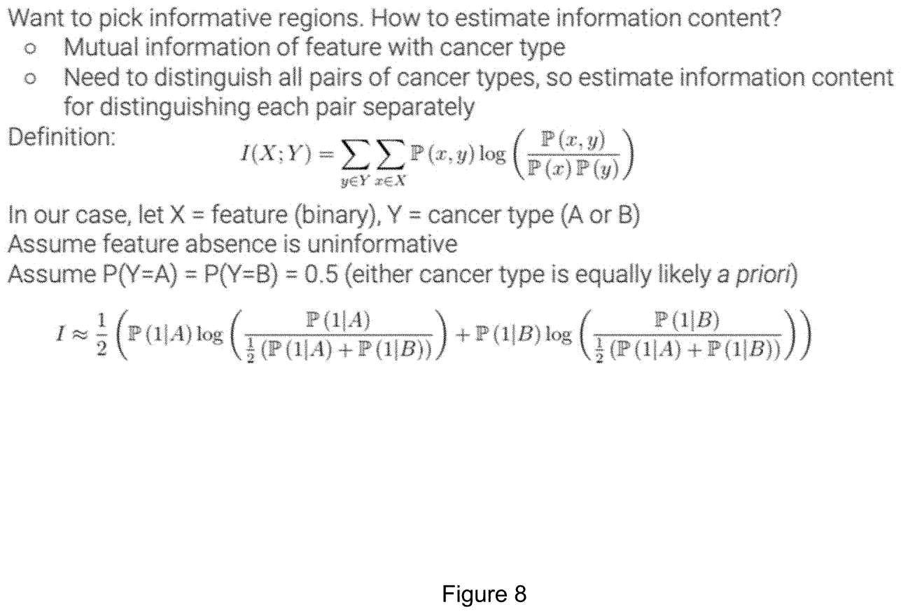

In some embodiments, the measure of association is a mutual information calculation. See, for example, Song et al., 2012, “Comparison of co-expression measures: mutual information, correlation, and model based indices,” BMC Bioinformatics 13, 328. For example in some embodiments the mutual information is calculated in accordance with FIG. 8. As described in FIG. 8, the mutual information between the training subject label Y (cancer type A or B in the case of two cancer types), and bin feature X is computed by mutual information. In fact, FIG. 8 provides a way of calculating mutual information under the assumption that the probability that a subject has either cancer type A or B is the same (P(Y=A)=P(Y=B) is the same. In some particular embodiments the measure of associate is mutual information calculated as:

$$I = {Σ}_{i , j} p ( {x}_{i} , {y}_{j} ) log \frac{p ( {x}_{i} , {y}_{j} )}{p ( {x}_{i} ) p ( {y}_{j} )} .$$

In some such embodiments, i and j are independent indices to the set of cancer conditions (e.g., first and second cancer condition). In some embodiments, xᵢ is the number of training subjects in the plurality of training subjects that have cancer condition i (e.g., where i is the first cancer condition or, alternatively, i is the second cancer condition, etc.). In some embodiments, yᵢ is the number of training subjects in the plurality of training subjects that have one or more cell-free fragments mapping to the respective bin that are assigned cancer condition j (e.g., where j is the first cancer condition or, alternatively, j is the second cancer condition, etc.). In the case of two cancer conditions, this measure of association has the form:

$$I = p ( {x}_{1} , {y}_{2} ) log \frac{p ( {x}_{1} , {y}_{2} )}{p ( {x}_{1} ) p ( {y}_{2} )} + p ( {x}_{2} , {y}_{1} ) log \frac{p ( {x}_{2} , {y}_{1} )}{p ( {x}_{2} ) p ( {y}_{1} )} + p ( {x}_{1} , {y}_{1} ) log \frac{p ( {x}_{1} , {y}_{1} )}{p ( {x}_{1} ) p ( {y}_{1} )} + \\ p ( {x}_{2} , {y}_{2} ) log \frac{p ( {x}_{2} , {y}_{2} )}{p ( {x}_{2} ) p ( {y}_{2} )} .$$

In some such embodiments, the measure of association is determined based on at least a) the number of training subjects that have the first cancer condition and also have one or more cell-free fragments in the respective bin assigned to the first cancer condition, b) the number of training subjects that have the first cancer condition but have one or more cell-free fragments in the respective bin assigned to the second cancer condition, c) the number of training subjects that have the second cancer condition and also have one or more cell-free fragments in the respective bin assigned to the second cancer condition, and d) the number of training subjects that have the second cancer condition but which have one or more cell-free fragments in the respective bin assigned to the first cancer condition.

In some embodiments, the function p(xᵢ,yⱼ) comprises

$$\frac{N ( {x}_{i} , {y}_{j} )}{{N}_{T}} ,$$

where N(xᵢ,yⱼ) is a number of training subjects in the plurality of training subjects that have the cancer condition i and also have one or more cell-free fragments mapping to the respective bin that are assigned the cancer condition j and N_{T} is the total number of training subjects in the plurality of training subjects. In some embodiments, the function p(xᵢ) comprises xᵢ/N_{T} (e.g., the ratio of the number of training subjects that have the i^{th} cancer condition in the total number of training subjects in the plurality of training subjects), and p(yⱼ) comprises yᵢ/N_{T} (e.g., the ratio of the number of training subjects that have the j^{th} cancer condition in the total number of training subjects in the plurality of training subjects).

In some embodiments, where there are two possible cancer conditions, the measure of association is a distance metric. Table 1 provides examples of such distance metrics:

TABLE 1
Example Distance Metrics

| Type | Distance Metric |
|---|---|
| Euclidean | $d ( {X}^{p} , {X}^{q} ) = \sqrt{{\sum }_{i = 1}^{n} {( {X}_{i}^{p} - {X}_{i}^{q} )}^{2}}$ |
| Manhattan distance | $d ( {X}^{p} , {X}^{q} ) = {\sum }_{i = 1}^{n} \vert {X}_{i}^{p} - {X}_{i}^{q} \vert$ |
| Maximum Value | d(X^{p}, X^{q}) = argmaxᵢ\|Xᵢ^{p} − Xᵢ^{q}\| |
| Normalized Euclidean | $d ( {X}^{p} , {X}^{q} ) = \sqrt{\frac{1}{n} {\sum }_{i = 1}^{n} {( \frac{{X}_{i}^{p} - {X}_{i}^{q}}{{max}_{i} - {min}_{i}} )}^{2}}$ |
| Normalized Manhattan | $d ( {X}^{p} , {X}^{q} ) = \frac{1}{n} {\sum }_{i = 1}^{n} \frac{\vert {X}_{i}^{p} - {X}_{i}^{q} \vert}{{max}_{i} - {min}_{i}}$ |
| Normalized Maximum Value | $d ( {X}^{p} , {X}^{q} ) = {argmax}_{i} \frac{\vert {X}_{i}^{p} - {X}_{i}^{q} \vert}{{max}_{i} - {min}_{i}}$ |
| Dice Coefficient | $d ( {X}^{p} , {X}^{q} ) = 1 - \frac{2 {\sum }_{i - 1}^{n} {X}_{i}^{p} {X}_{i}^{q}}{{\sum }_{i - 1}^{n} {X}_{i}^{{p}^{2}} + {\sum }_{i - 1}^{n} {X}_{i}^{{q}^{2}}}$ |
| Cosine distance | $d ( {X}^{p} , {X}^{q} ) = 1 - \frac{{\sum }_{i - 1}^{n} {X}_{i}^{p} {X}_{i}^{q}}{{\sum }_{i - 1}^{n} {X}_{i}^{{p}^{2}} \cdot {\sum }_{i - 1}^{n} {X}_{i}^{{q}^{2}}}$ |
| Jaccard coefficient | $d ( {X}^{p} , {X}^{q} ) = 1 - \frac{{\sum }_{i - 1}^{n} {X}_{i}^{p} {X}_{i}^{q}}{{\sum }_{i - 1}^{n} {X}_{i}^{{p}^{2}} + {\sum }_{i - 1}^{n} {X}_{i}^{{q}^{2}} - {\sum }_{i - 1}^{n} {X}_{i}^{p} {X}_{i}^{q}}$ |

In Table 1, X^{p}=[X₁^{p}, . . . , Xₙ^{p}] is a training dataset state vector, in which each respective element in [X₁^{p}, . . . , Xₙ^{p}] represents a training subject cancer indication of a corresponding cancer subject in the plurality of training subjects and n represent the n subject of the training population. For instance, in some embodiments, a given element X₁^{p} is “0” when the training subject has the first cancer condition and is zero when the training subject has the second cancer condition. In Table 1, X^{q}=[X₁^{q}, . . . , Xₙ^{q}] is a is vector for a respective bin for which the distance metric is computed. Like X^{p}, each element of X^{q} represents a corresponding cancer condition. However, for X^{q} each respective element in [X₁^{q}, . . . , Xₙ^{q}] represents a measured aspect of the respective bin of the training subject for which the distance metric is computed. In some embodiments, each element in [X₁^{q}, . . . , Xₙ^{q}] is a binary indication as to whether any of the fragments in the subject bin have been classified as being of the first cancer condition (e.g., “0” when there are, “1” when there are not). In some embodiments, each element in [X₁^{q}, . . . , Xₙ^{q}] is a binary indication as to whether any of the fragments in the subject bin have been classified as being of the second cancer condition (e.g., “0” when there are, “1” when there are not). In some embodiments, each element in [X₁^{q}, . . . , Xₙ^{q}] is a ratio of the number of fragments in the subject bin that have been classified as being of the first cancer condition (e.g., “0” when there are, “1” when there are not) divided by all the fragments in the bin. In some embodiments, each element in [X₁^{q}, . . . , Xₙ^{q}] is a ratio of the number of fragments in the subject bin that have been classified as being of the second cancer condition (e.g., “0” when there are, “1” when there are not) divided by all the fragments in the bin. In some embodiments, each element in [X₁^{q}, . . . , Xₙ^{q}] is a ratio of the number of fragments in the subject bin that have been classified as being of the first cancer condition (e.g., “0” when there are, “1” when there are not) divided by all the fragments in the subject bin that have been classified as being of the second cancer condition. In some embodiments, each element in [X₁^{q}, . . . , Xₙ^{q}] is a binary indication as to whether a threshold presence of the fragments in the subject bin that have been classified as being of the first cancer condition (e.g., “0” when the threshold is satisfied, “1” when the threshold is not satisfied). This threshold can be a threshold of any of the above described ratios or fragment counts. Further, in Table 1, maxᵢ and minᵢ are the maximum value (e.g., “1”) and the minimum value (e.g., “0”) of an i^{th} element, respectively. Additional details and information regarding distance based classification are disclosed in Yang et al., 1999, “DistAI: An Inter-pattern Distance-based Constructive Learning Algorithm,” Intelligent Data Analysis, 3(1), 55-83, which is hereby incorporated by reference.

In some embodiments, the calculation of the measure of association determines a measure of association for each bin in the plurality of bins where each training subject in the plurality of training subjects has one of a plurality of cancer conditions. In some such embodiments, the measure of association is calculated as:

$$I = {\sum }_{i , j…n} p ( {x}_{i} , {y}_{j} , … , {z}_{n} ) log \frac{p ( {x}_{i} , {y}_{j} , … , {z}_{n} )}{p ( {x}_{i} ) p ( {y}_{j} ) … p ( {z}_{n} )} .$$

In some embodiments, 1,1, and n in this equation are independent indices to the set of cancer conditions (e.g., to each respective cancer condition in the plurality of cancer conditions). In some embodiments, xᵢ is the number of training subjects in the plurality of training subjects that have cancer condition i. In some embodiments, yⱼ is a number of training subjects in the plurality of training subjects that have one or more cell-free fragments mapping to the respective bin that are assigned cancer condition j. There is a respective number of training subjects in the plurality of training subjects that have each respective cancer condition, up to an including zₙ. In some embodiments, the function p(xⱼ, yⱼ, . . . zₙ) comprises the ratio

$$\frac{N ( {x}_{i} , {y}_{j} , … , {z}_{n} )}{{N}_{T}} ,$$

where N(xᵢ, yⱼ, . . . , zₙ) is a number of training subjects in the plurality of training subjects that have the cancer condition i and also have one or more cell-free fragments mapping to the respective bin that are assigned to one of the cancer conditions j through n, and N_{T} is the total number of training subjects in the plurality of training subjects. In some embodiments, the function p(xᵢ) comprises xᵢ/N_{T} (e.g., the ratio of the number of training subjects that have the i^{th} cancer condition in the total number of training subjects in the plurality of training subjects), and p(yⱼ) comprises yⱼ/N_{T} (e.g., the ratio of the number of training subjects that have the j^{th} cancer condition in the total number of training subjects in the plurality of training subjects). In some embodiments, each cancer condition in the plurality of cancer conditions has a corresponding ratio (e.g., p(zₙ)) of the number of training subjects that have the respective cancer condition (e.g., the n^{th} cancer condition).

Block 228. The method continues, referring to Block 228 of FIG. 2B, by identifying the plurality of features for estimating subject cell source fraction as a subset of the plurality of bins, where each respective bin in the subset of the plurality of bins satisfies a selection criterion based on the corresponding measure of association for the respective bin.

In some embodiments, the selection criterion specifies selection of the bins having one of the top N measures of association, where N is a positive integer of 50 or greater. In some embodiments, N is between 500 and 5000. In some embodiments, N is between 800 and 1500. In some embodiments, N is at least 100, at least 200, at least 300, at least 400, at least 500, at least 600, at least 700, at least 800, at least 900, at least 1000, at least 1100, at least 1200, at least 1300, at least 1400, or at least 1500.

In some embodiments, referring to Block 230, the selection criteria specifies selection of bins having one of the top N measures of association, where N is a positive integer of 50 or greater (e.g., at least 50 bins with the highest measures of association are selected as features).

In some embodiments, the plurality of features comprises at least 10, at least 50, at least 100, at least 200, at least 300, at least 400, at least 500, at least 600, at least 700, at least 800, at least 900, at least 1000, at least 1100, at least 1200, at least 1300, at least 1400, or at least 1500 features. In some embodiments, the plurality of features comprises between 500 and 5000, between 800 and 1500, or more than 1500 features.

Estimating Cell Source Fractions.

In some embodiments, after identifying a plurality of features (e.g., a subset of bins) for estimating subject cell source fraction, the method further comprises estimating a cell source fraction for a test subject based on at least the plurality of features.

In some embodiments, the method performs cell source or tumor fraction estimation by a procedure that comprises obtaining, in electronic form, a corresponding methylation pattern of each respective cell-free fragment in a test plurality of cell-free fragments (e.g., from the test subject for which cancer classification is desired), where the corresponding methylation pattern of each respective cell-free fragment (i) is determined by a methylation sequencing of one or more nucleic acid samples comprising the respective fragment in a biological sample obtained from the test subject and (ii) comprises a methylation state of each CpG site in a corresponding plurality of CpG sites in the respective fragment. The procedure further comprises mapping each cell-free fragment in the test plurality of cell-free fragments to a bin in the plurality of bins, thereby obtaining a plurality of test sets of cell-free fragments, each test set of cell-free fragments mapped to a different bin in the plurality of bins. The procedure continues by assigning a cell-free fragment cancer condition for each respective cell-free fragment in each test set of cell-free fragments the plurality of test sets of cell-free fragments as the function of a function of an output of the classifier upon inputting a methylation pattern of the respective cell-free fragment into the classifier. The procedure comprises computing a first measure of central tendency of the number of cell-free fragments from the test subject that have been assigned the first cancer condition in each test set of cell-free fragments across the subset of the plurality of bins and computing a second measure of central tendency of the number of cell-free fragments from the test subject in each test set of cell-free fragments across the subset of the plurality of bins. The procedure estimates the cell source fraction for the test subject using the first measure of central tendency and the second measure of central tendency.

In some embodiments, the second cancer condition comprises an absence of cancer, and the cell source fraction estimated for the test subject comprises a tumor fraction for the test subject.

For instance, in some embodiments, tumor fraction estimates are calculated based on the assumption that one or more methylation state patterns in a biological sample of the test subject (e.g., cfDNA and/or plasma) are tumor-derived, and that the frequency of such tumor-derived methylation patterns are directly proportional to the fraction of cancer cells to normal cells (e.g., the tumor fraction).

There are various methods of determining such fractions, some of which are described in U.S. patent application Ser. No. 16/719,902, entitled “Systems and Methods for Estimating Cell Source Fractions using Methylation Information,” filed Dec. 18, 2019 and U.S. patent application Ser. No. 16/850,634 entitled “Systems and Methods for Tumor Fraction Estimation from Small Variants,” filed Apr. 16, 2020, both of which are hereby incorporated herein by reference in their entireties.

In some embodiments, the first measure of central tendency is an arithmetic mean, a weighted mean, a midrange, a midhinge, a trimean, a Winsorized mean, a mean, or a mode of the number of cell-free fragments from the plurality of test subjects that have been assigned the first cancer condition in each test set of cell-free fragments across the subset of the plurality of bins. In some embodiments, the second measure of central tendency is an arithmetic mean, a weighted mean, a midrange, a midhinge, a trimean, a Winsorized mean, a mean, or a mode of the number of cell-free fragments from the plurality of test subjects in each test set of cell-free fragments across the subset of the plurality of bins. In some embodiments, estimating the cell source fraction comprises dividing the first measure of central tendency by the second measure of central tendency. In some embodiments, the respective subject cancer condition for each training subject in the plurality of training subjects is selected from a plurality of cancer conditions. In some embodiments, a corresponding measure of central tendency is determined for each respective cancer condition in the plurality of cancer conditions. In some such embodiments, estimating the cell source fraction comprises dividing the first measure of central tendency by the sum of each other measure of central tendency.

In some embodiments, the tumor fraction of the test subject is between 0.003 and 1.0. In some embodiments, the tumor fraction of the test subject is in the range of 0.001 and 1.0. In some embodiments, the tumor fraction of the subject is at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1.0.

In some embodiments, determining the cell source (e.g., tumor) fraction of the subject further identifies a cancer of origin of the subject. In some embodiments, the first and/or second cancer condition comprises a tissue of origin (e.g., where a cancer is believed to originate). In some embodiments, the first and/or second cancer condition comprises a stage of a cancer (e.g., stage I, II, III or IV).

In some embodiments, the cancer of origin comprises a first cancer condition selected from the group consisting of non-cancer, breast cancer, lung cancer, prostate cancer, colorectal cancer, renal cancer, uterine cancer, pancreatic cancer, cancer of the esophagus, lymphoma, head/neck cancer, ovarian cancer, hepatobiliary cancer, melanoma, cervical cancer, multiple myeloma, leukemia, thyroid cancer, bladder cancer, gastric cancer, nasopharyngeal cancer, liver cancer, or a combination thereof.

In some embodiments, the cancer of origin comprises at least a first cancer condition and a second cancer condition each selected from the group consisting of breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, nasopharyngeal cancer, liver cancer, or a combination thereof.

In some embodiments, the first and/or second cancer condition comprises a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, a stage of a gastric cancer, a stage of nasopharyngeal cancer, a stage of liver cancer, or a combination thereof.

In some embodiments, determining the cell source (e.g., tumor) fraction of the test subject further includes providing a treatment recommendation (e.g., a cancer treatment) to the test subject, where the treatment recommendation is based at least in part on the cell source fraction (e.g., how progressed the disease is) and the cancer of origin.

In some embodiments, the method further comprises determining the cell source (e.g., tumor) fraction of the test subject at one or more time points (e.g., before or after treatment) to monitor disease progression or to monitor treatment effectiveness (e.g., therapeutic efficacy). For example, in some embodiments, in increase in tumor fraction over time (e.g., at a second, later time point) indicates disease progression, and conversely, in some embodiments, a decrease in tumor fraction over time (e.g., at a second, later time point) indicates successful treatment.

For example, in some embodiments, the method further comprises applying a treatment regimen to the test subject based at least in part, on a value of the cell source fraction for the test subject. In some embodiments, the treatment regimen comprises applying an agent for cancer to the test subject. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, or Bortezomib, or generic equivalents thereof.

In some embodiments, the test subject has been treated with an agent for cancer and the method further comprises using the cell source fraction for the test subject to evaluate a response of the test subject to the agent for cancer. In some embodiments, the agent for cancer is a hormone, an immune therapy, radiography, or a cancer drug. In some embodiments, the agent for cancer is Lenalidomid, Pembrolizumab, Trastuzumab, Bevacizumab, Rituximab, Ibrutinib, Human Papillomavirus Quadrivalent (Types 6, 11, 16, and 18) Vaccine, Pertuzumab, Pemetrexed, Nilotinib, Nilotinib, Denosumab, Abiraterone acetate, Promacta, Imatinib, Everolimus, Palbociclib, Erlotinib, Bortezomib, Bortezomib, or generic equivalents thereof.

In some embodiments, the test subject has been treated with an agent for cancer and the method further comprises using the cell source fraction for the test subject to determine whether to intensify or discontinue the agent for cancer in the test subject. In some embodiments, the test subject has been subjected to a surgical intervention to address the cancer and the method further comprises using the cell source fraction for the test subject to evaluate a condition of the test subject in response to the surgical intervention.

In some embodiments, the method is repeated at each respective time point in a plurality of time points (e.g., two or more time points, three or more time points four or more time points) across an epoch, thereby obtaining a corresponding cell source (e.g., tumor) fraction, in a plurality of cell source (e.g., tumor) fractions, for the test subject at each respective time point and using the plurality of cell source (e.g., tumor) fractions to determine a state or progression of a disease condition in the test subject during the epoch in the form of an increase or decrease of the first cell source (e.g., tumor) fraction over the epoch.

In some such embodiments, the epoch is a period of months and each time point in the plurality of time points is a different time point in the period of months. In some embodiments, the period of months is between 1 and 4 months, between 4 and 8 months, between 8 and 12 months, between 12 and 18 months, between 18 and 24 months, or more than 24 months. In some embodiments, the period of months is less than four months.

In some embodiments, the epoch is a period of years and each time point in the plurality of time points is a different time point in the period of years. In some embodiments, the period of years is between two and ten years. In some embodiments, the period of years is between 1 and 5 years, between 5 and 10 years, between 10 and 15 years, between 15 and 20 years, or more than 20 years.

In some embodiments, the epoch is a period of hours and each time point in the plurality of time points is a different time point in the period of hours. In some embodiments, the period of hours is between one hour and six hours. In some embodiments, the period of hours is between 1 and 3 hours, between 3 and 6 hours, between 6 and 9 hours, between 9 and 12 hours, between 12 and 18 hours, between 18 and 24 hours, or more than 24 hours.

In some embodiments, the method further comprises changing a diagnosis of the test subject when the first cell source (e.g., tumor) fraction of the subject is observed to change by a threshold amount across the epoch. In some embodiments, the method further comprises changing a prognosis of the subject when the first cell source (e.g., tumor) fraction of the subject is observed to change by a threshold amount across the epoch. In some embodiments, the method further comprises changing a treatment of the subject when the first cell source (e.g., tumor) fraction of the subject is observed to change by a threshold amount across the epoch. In some of the forgoing embodiments, the threshold is greater than one percent, greater than 5 percent, greater than ten percent, greater than twenty percent, greater than thirty percent, greater than forty percent, or greater than fifty percent. In some embodiments, the threshold is greater than two-fold, greater than three-fold, greater than four-fold, or greater than five-fold.

In certain embodiments, the method is conducted at a first time point that is before a cancer treatment (e.g., before a resection surgery or a therapeutic intervention) as well as at a second time point that is after a cancer treatment (e.g., after a resection surgery or therapeutic intervention), and the disclosed methods are used to monitor the effectiveness of the treatment by comparison of the cell source (e.g., tumor) fraction determined by the disclosed methods at each time point. For example, if the tumor fraction at the second time point decreases compared to the tumor fraction at the first time point, then the treatment is deemed successful. However, if the tumor fraction at the second time point increases compared to the tumor fraction at the first time point, then the treatment is deemed not successful. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, biological samples (cfDNA samples) may be obtained from a test subject (e.g., a cancer patient) at a first and second time point and analyzed, e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that biological samples can be obtained from a test subject (e.g., a cancer patient) over any number of time points and analyzed in accordance with the methods of the disclosure to monitor a cancer condition (e.g., via tumor fraction) in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, biological samples can be obtained from the patient at least once every 3 months, at least once every 6 months, at least once a year, at least once every 2 years, at least once every 3 years, at least once every 4 years, or at least once every 5 years.

Determining an Estimated Cell Source Fraction for a Test Subject.

Block 302. Referring to Block 302 of FIG. 3A, a method of estimating cell source fraction for a subject (e.g., a test subject) is provided. In some embodiments the subject is human. In some embodiments, a subject is a male or female of any stage (e.g., a man, a woman or a child). In some embodiments, the cell source fraction for a subject is derived from a single cell source. In some embodiments, the cell source fraction for a subject is derived from two or more cell sources. In some embodiments, the cell source fraction is as described with regards to Block 202 above.

Block 304. Referring to Block 304, the method continues by obtaining, in electronic form, a corresponding methylation pattern of each respective cell-free fragment in a plurality of cell-free fragments (e.g., the plurality of cell-free fragments are derived from a biological sample of the subject), where the corresponding methylation pattern of each respective cell-free fragment (i) is determined by a methylation sequencing of one or more nucleic acid samples comprising the respective fragment in a biological sample obtained from the subject and (ii) comprises a methylation state of each CpG site in a corresponding plurality of CpG sites in the respective fragment. In some embodiments, referring to Block 306, the plurality of cell-free fragments has an average length of less than 500 nucleotides. In some embodiments, the cell-free fragments are derived from the biological sample as described above with regards to Block 204.

In some embodiments, the biological sample comprises or consists of blood, whole blood, plasma, serum, urine, cerebrospinal fluid, fecal, saliva, sweat, tears, pleural fluid, pericardial fluid, or peritoneal fluid of the subject. In such embodiments, the biological sample may include the blood, whole blood, plasma, serum, urine, cerebrospinal fluid, fecal, saliva, sweat, tears, pleural fluid, pericardial fluid, or peritoneal fluid of the subject as well as other components (e.g., solid tissues, etc.) of the subject.

Such biological samples contain cell-free nucleic acid fragments (e.g., cfDNA fragments). In some embodiments, the biological sample is processed to extract the cell-free nucleic acids in preparation for sequencing analysis. By way of a non-limiting example, in some embodiments, cell-free nucleic acid fragments are extracted from a biological sample (e.g., blood sample) collected from a subject in K2 EDTA tubes. In the case where the biological samples are blood, the samples are processed within two hours of collection by double spinning of the biological sample first at ten minutes at 1000 g, and then the resulting plasma is spun ten minutes at 2000 g. The plasma is then stored in 1 ml aliquots at −80° C. In this way, a suitable amount of plasma (e.g., 1-5 ml) is prepared from the biological sample for the purposes of cell-free nucleic acid extraction. In some such embodiments cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton, et al., 2017, “Phylogenetic ctDNA analysis depicts early stage lung cancer evolution,” Nature, 545(7655): 446-451, which is hereby incorporated by reference. Other equivalent methods can be used to prepare cell-free nucleic acid from biological methods for the purpose of sequencing, and all such methods are within the scope of the present disclosure.

In some embodiments, the cell-free nucleic acid fragments that are obtained from a biological sample are any form of nucleic acid defined in the present disclosure, or a combination thereof. For example, in some embodiments, the cell-free nucleic acid that is obtained from a biological sample is a mixture of RNA and DNA.

In some embodiments, the cell-free nucleic acid fragments are treated to convert unmethylated cytosines to uracils. In one embodiment, the method uses a bisulfite treatment of the DNA that converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cell-free nucleic acid fragments, a sequencing library is prepared. Optionally, the sequencing library is enriched for cell-free nucleic acid fragments, or genomic regions, that are informative for cell origin using a plurality of hybridization probes. The hybridization probes are short oligonucleotides that hybridize to particularly specified cell-free nucleic acid fragments, or targeted regions, and enrich for those fragments or regions for subsequent sequencing and analysis. In some embodiments, hybridization probes are used to perform a targeted, high-depth analysis of a set of specified CpG sites that are informative for cell origin. Once prepared, the sequencing library or a portion thereof is sequenced to obtain a plurality of sequence reads.

In some embodiments, the sequencing comprises methylation sequencing. In some embodiments, the methylation sequencing is paired-end sequencing. In some embodiments, the methylation sequencing is single-read sequencing. In some embodiments, the methylation sequencing is whole genome methylation sequencing. In some embodiments, the methylation sequencing is targeted sequencing using a plurality of nucleic acid probes and each respective bin in the plurality of bins is associated with at least one corresponding nucleic acid probe in the plurality of nucleic acid probes. In some embodiments, each respective bin in the plurality of bins is associated with at least two corresponding nucleic acid probes in the plurality of nucleic acid probes.

In some embodiments, the plurality of nucleic acid probes (e.g., probes used for targeted sequencing) comprises 1,000 or more nucleic acid probes, 2,000 or more nucleic acid probes, 3,000 or more nucleic acid probes, 4,000 or more nucleic acid probes, 5,000 or more nucleic acid probes, 10,000 or more nucleic acid probes, 20,000 or more nucleic acid probes or 30,000 or more nucleic acid probes. In some embodiments, the plurality of nucleic acid probes between 1,000 nucleic acid probes and 30,000 nucleic acid probes.

In some embodiments, wherein the methylation sequencing (e.g., as performed in accordance with any methylation sequencing method described herein or known in the art) detects one or more 5-methylcytosine (5mC) and/or 5-hydroxymethylcytosine (5hmC) in the respective fragment.

In some embodiments, the methylation sequencing comprises conversion of one or more unmethylated cytosines or one or more methylated cytosines, in sequence reads of the respective fragment, to a corresponding one or more uracils. In some embodiments, the one or more uracils are detected during the methylation sequencing as one or more corresponding thymines. In some embodiments, the conversion of one or more unmethylated cytosines or one or more methylated cytosines comprises a chemical conversion, an enzymatic conversion, or combinations thereof.

In some embodiments, the methylation stat of a respective CpG site in the corresponding plurality of CpG sites in the respective fragment is: a) methylated when the respective CpG site is determined by the methylation sequencing to be methylated, b) unmethylated when the respective CpG site is determined by the methylation sequencing to not be methylated, and c) flagged as “other” when the methylation sequencing is unable to call the methylation state of the respective CpG site as methylation or unmethylated.

Block 308. Referring to Block 308, the method continues by mapping each cell-free fragment in the plurality of cell-free fragments to a bin in a plurality of bins, thereby obtaining a plurality of sets of cell-free fragments, each set of cell-free fragments mapped to a different bin in the plurality of bins.

In some embodiments, referring to Block 310, the plurality of bins consists of between 1000 and 100,000 bins. In some embodiments, the plurality of bins consists of between 15,000 and 80,000 bins. In some embodiments, the plurality of bins consists of any number of bins as described with regards to Block 210 above.

Referring to Block 312, in some embodiments, each respective in in the plurality of bins has, on average, between 10 and 1200 residues. In some embodiments, each respective bin in the plurality of bins has on average between 10 and 10,000 residues. In some embodiments, each respective bin in the plurality of bins has, on average, between 10 and 500 residues. In some embodiments, each respective bin in the plurality of bins has, on average, between 10 and 100 residues. In some embodiments, each respective bin in the plurality of bins has, on average, between 25 and 100 residues. In some embodiments, each respective bin in the plurality of bins has, on average, between 5000 and 10,000 residues.

In addition, with regards to Block 314, in some embodiments, each bin in the plurality of bins comprises or consists of 2, 3, 4, 5, 6, 7, 8, 9, 10, 11, 12, 13, 14, 15, 16, 17, 18, 19, 20 or more CpG sites. In some embodiments, each bin in the plurality of bins consists of between 2 and 100 contiguous CpG sites in a human reference genome. In some embodiments, each bin in the plurality of bins consist of between 2 and 50 contiguous CpG sites. In some embodiments, each bin in the plurality of bins consists of between 50 and 100 contiguous CpG sites. In some embodiments, each bin in the plurality of bins consists of at least 2 contiguous CpG sites.

Block 316. Referring to Block 316, the method continues by assigning a cell-free fragment cancer condition to each respective cell-free fragment in each training set of cell-free fragments in the plurality of training sets of cell-free fragments, where the cell-free fragment cancer condition is one of the first cancer condition and the second cancer condition, as a function of an output of a classifier upon inputting a methylation pattern of the respective cell-free fragment into the classifier. Referring to Block 318, in some embodiments, the first cancer condition is cancer and the second cancer condition is absence of cancer. In some embodiments, the first cancer condition is cancer and the second cancer condition is absence of cancer. In some embodiments, the cell-free fragment cancer condition is one of a plurality of cancer conditions (e.g., as described above with reference to Block 206).

In some embodiments, the classifier used for assigning a cell-free fragment condition comprises a first model for the first cancer condition and a second model for the second cancer condition, where the first model is a first mixture model comprising a first plurality of sub-models, the second model is a second mixture model comprising a second plurality of sub-models, and each sub-model in the first and second plurality of sub-models represents an independent corresponding methylation model for a source of cell-free fragments in the corresponding biological sample. In some embodiments, the classifier has the form of equations (1) or equation (3).

Block 320. Referring to Block 320 of FIG. 3B, the method further comprises computing a first measure of central tendency of the number of cell-free fragments from the subject that have been assigned the first cancer condition in each set of cell-free fragments across the plurality of bins. In some embodiments, referring to Block 322, the first measure of central tendency is an arithmetic mean, a weighted mean, a midrange, a midhinge, a trimean, a Winsorized mean, a mean, or a mode of the number of cell-free fragments from the subject that have been assigned the first cancer condition in each set of cell-free fragments across the plurality of bins.

Block 324. Referring to Block 324, the method further comprises computing a second measure of central tendency of the number of cell-free fragments from the subject that have been assigned the second cancer condition in each set of cell-free fragments across the plurality of bins. In some embodiments, referring to Block 326, the second measure of central tendency is an arithmetic mean, a weighted mean, a midrange, a midhinge, a trimean, a Winsorized mean, a mean, or a mode of the number of cell-free fragments from the subject that have been assigned the first cancer condition in each set of cell-free fragments across the plurality of bins.

Block 328. Referring to Block 328, the method proceeds by estimating the cell source fraction for the subject using the first measure of central tendency and the second measure of central tendency. In some embodiments, the cell source fraction comprises a tumor fraction. Regarding Block 330, in some embodiments, estimating the tumor fraction comprises dividing the first measure of central tendency by the second measure of central tendency.

In some embodiments, the cell source fraction is used as a basis or a partial basis for determining a treatment option for treating a disease (e.g., a cancer) associated with the cell source in the test subject. In some embodiments, the cell source fraction is used as a basis for treatment monitoring. In some embodiments, given the estimated cell source fraction of the subject, it is possible to determine that certain treatment options are not being effective or will not be effective for the subject. For example, checkpoint immunotherapy will not be effective if cytotoxic T-cells are dysfunctional and undergo apoptosis. Such a situation is indicated, for example, when a plurality of fragments from the biological sample of the subject is determined to originate from cytotoxic T-cells in the blood. In some embodiments, the estimated cell source fraction aids in monitoring minimum residual disease amount.

One skilled in the art will recognize that any of the embodiments disclosed in the preceding sections (see, for example, “Identifying features for estimating cell source fraction”) are applicable in any combination to the methods and embodiments for determining an estimated cell source fraction for a test subject, as described herein.

### EXAMPLES

### Example 1—Increase in Median ctDNA Fraction by Cancer by Stage

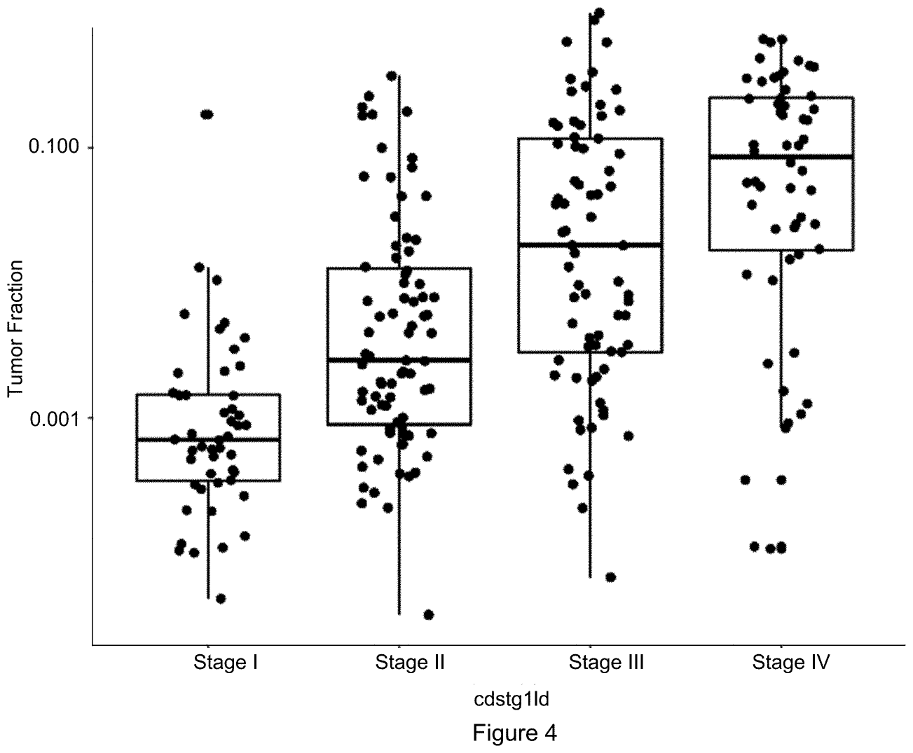

Referring to FIG. 4, subjects are grouped by cancer stages I, II, III, and IV, regardless of the type of cancer that they have. In FIG. 4, the x-axis indicates which cancer stage each subject has and while the y-axis indicates the observed ctDNA fraction for each subject. The method used to compute the cfDNA fraction for each subject comprises obtaining a first plurality of nucleic acid fragment sequence in electronic form from a biological sample of each subject in a cohort, where the biological sample comprises cell-free nucleic acid molecules.

FIG. 4 provides an analysis of how ctDNA fraction varies by cancer stage regardless of cancer type, among subjects that have cell-free sequence reads that indicate their underlying cancer. FIG. 4 thus shows that, as the disease is more severe as determined by clinically staging (stages 1 through 4), more evidence of cell source fraction (larger ctDNA fraction) is found in the cfDNA. While FIG. 4 shows that while this is the general case across the CCGA cohort (see Example 3 for details of the CCGA cohort), there are violations (outliers) to this trend. Such outliers in FIG. 4 are suggestive and best explained by clinical misclassification. FIG. 4 thus shows a fundamental component of the underlying disease, which is general expected cell source fraction rates in the cfDNA. FIG. 4 also shows that stage 4 has some individuals that have very low shedding rates indicating that there are different sub-states within stage 4.

FIG. 4 illustrates that shedding rates (ctDNA fraction) can be used as a basis for establishing meaningful and informative thresholds.

### Example 2—Obtaining a Plurality of Sequence Reads

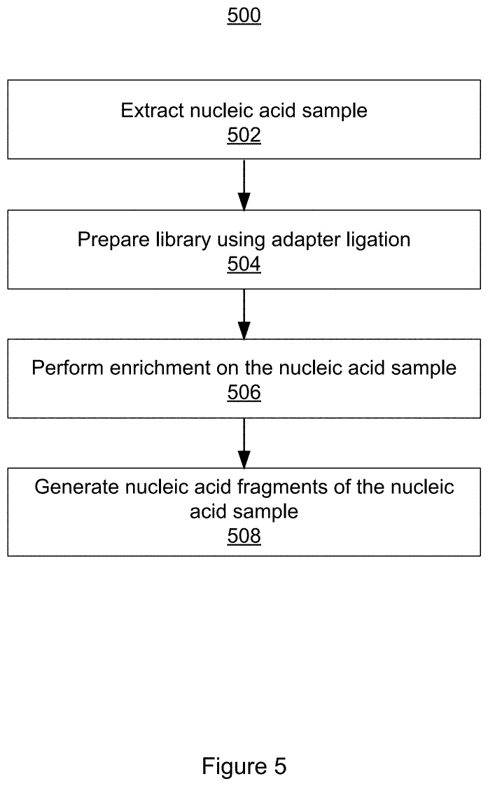

FIG. 5 is a flowchart of method 500 for preparing a nucleic acid sample for sequencing according to one embodiment. The method 500 includes, but is not limited to, the following steps. For example, any step of method 500 may comprise a quantitation sub-step for quality control or other laboratory assay procedures known to one skilled in the art.

In Block 502, a nucleic acid sample (DNA or RNA) is extracted from a subject. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In Block 504, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment. This provides a way to identify sequence reads that came from the same original fragment in downstream analysis.

In Block 506, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer class or tissue of origin). For a given workflow, the probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a methylation site panel. In one embodiment, the probes are designed based on a panel of targeted genes to analyze particular mutations or target regions of the genome (e.g., of the human or another organism) that are suspected to correspond to certain cancers or other types of diseases. Moreover, the probes may cover overlapping portions of a target region. In Block 408, these probes are used to general sequence reads of the nucleic acid sample.

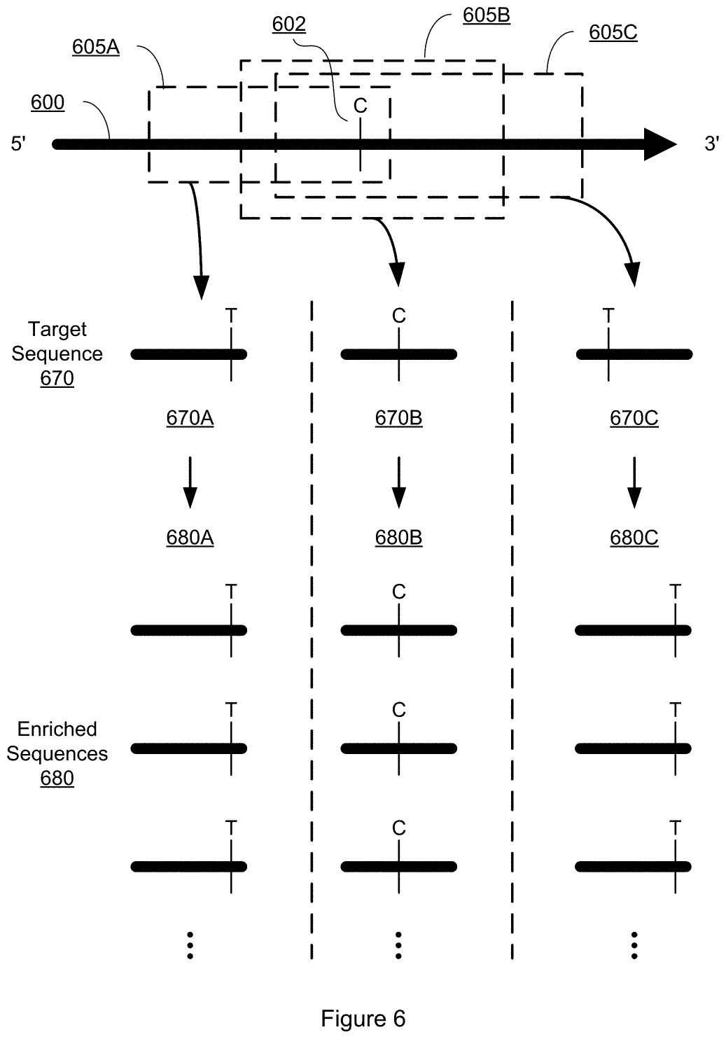

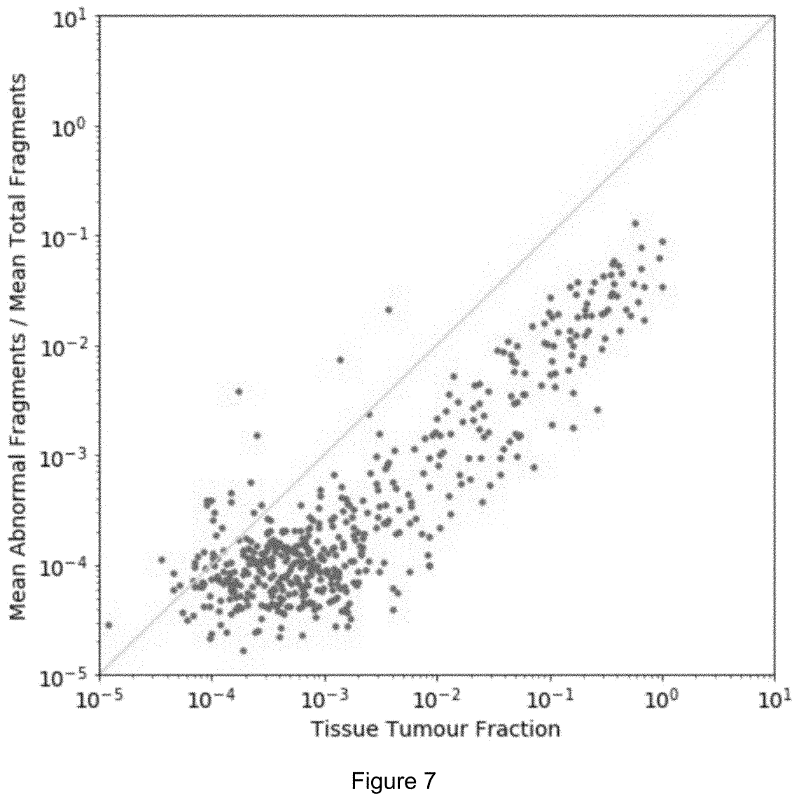

FIG. 6 is a graphical representation of the process for obtaining sequence reads according to one embodiment. FIG. 6 depicts one example of a nucleic acid segment 800 from the sample. Here, the nucleic acid segment 600 can be a single-stranded nucleic acid segment, such as a single stranded. In some embodiments, the nucleic acid segment 600 is a double-stranded cfDNA segment. The illustrated example depicts three regions 605A, 605B, and 605C of the nucleic acid segment that can be targeted by different probes. Specifically, each of the three regions 605A, 605B, and 605C includes an overlapping position on the nucleic acid segment 600. An example overlapping position is depicted in FIG. 5 as the cytosine (“C”) nucleotide base 602. The cytosine nucleotide base 602 is located near a first edge of region 605A, at the center of region 605B, and near a second edge of region 605C.

In some embodiments, one or more (or all) of the probes are designed based on a gene panel or methylation site panel to analyze particular mutations or target regions of the genome (e.g., of the human or another organism) that are suspected to correspond to certain cancers or other types of diseases. By using a targeted gene panel or methylation site panel rather than sequencing all expressed genes of a genome, also known as “whole exome sequencing,” the method 600 may be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 600 using one or more probes results in an understanding of a target sequence 670. As shown in FIG. 6, the target sequence 670 is the nucleotide base sequence of the region 605 that is targeted by a hybridization probe. The target sequence 670 can also be referred to as a hybridized nucleic acid fragment. For example, target sequence 670A corresponds to region 605A targeted by a first hybridization probe, target sequence 670B corresponds to region 605B targeted by a second hybridization probe, and target sequence 670C corresponds to region 605C targeted by a third hybridization probe. Given that the cytosine nucleotide base 602 is located at different locations within each region 605A-C targeted by a hybridization probe, each target sequence 670 includes a nucleotide base that corresponds to the cytosine nucleotide base 602 at a particular location on the target sequence 670.

After a hybridization step, the hybridized nucleic acid fragments are captured and may also be amplified using PCR. For example, the target sequences 670 can be enriched to obtain enriched sequences 680 that can be subsequently sequenced. In some embodiments, each enriched sequence 680 is replicated from a target sequence 670. Enriched sequences 680A and 680C that are amplified from target sequences 670A and 670C, respectively, also include the thymine nucleotide base located near the edge of each sequence read 680A or 680C. As used hereafter, the mutated nucleotide base (e.g., thymine nucleotide base) in the enriched sequence 680 that is mutated in relation to the reference allele (e.g., cytosine nucleotide base 602) is considered as the alternative allele. Additionally, each enriched sequence 680B amplified from target sequence 670B includes the cytosine nucleotide base located near or at the center of each enriched sequence 680B.

In Block 508 of FIG. 5, sequence reads are generated from the enriched DNA sequences, e.g., enriched sequences 680 shown in FIG. 6. Sequencing data may be acquired from the enriched DNA sequences by known means in the art. For example, the method 600 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as methylation state determination.

### Example 3—Cell-Free Genome Atlas Study (CCGA) Cohort

Subjects from the CCGA [NCT02889978] were used in the Examples of the present disclosure. CCGA is a prospective, multi-center, observational cfDNA-based early cancer detection study that has enrolled over 15,000 demographically-balanced participants at over 140 sites.

This example looks at one of the sub-studies of CCGA. Blood was collected from subjects with newly diagnosed therapy-naive cancer (C, case) and participants without a diagnosis of cancer (noncancer [NC], control) as defined at enrollment. This preplanned substudy included 878 cases, 580 controls, and 169 assay controls (n=1627) across twenty tumor types and all clinical stages.

All samples were analyzed by: 1) paired cfDNA and white blood cell (WBC)-targeted sequencing (60,000X, 507 gene panel); a joint caller removed WBC-derived somatic variants and residual technical noise; 2) paired cfDNA and WBC whole-genome sequencing (WGS; 35X); a novel machine learning algorithm generated cancer-related signal scores; joint analysis identified shared events; and 3) cfDNA whole-genome bisulfite sequencing (WGBS; 34X); normalized scores were generated using abnormally methylated fragments. In the targeted assay, non-tumor WBC-matched cfDNA somatic variants (SNVs/indels) accounted for 76% of all variants in NC and 65% in C. Consistent with somatic mosaicism (e.g., clonal hematopoiesis), WBC-matched variants increased with age; several were non-canonical loss-of-function mutations not previously reported. After WBC variant removal, canonical driver somatic variants were highly specific to C (e.g., in EGFR and PIK3CA, 0 NC had variants vs 11 and 30, respectively, of C). Similarly, of 8 NC with somatic copy number alterations (SCNAs) detected with WGS, four were derived from WBCs. WGBS data of the CCGA reveals informative hyper- and hypo-fragment level CpGs (1:2 ratio); a subset of which was used to calculate methylation scores. A consistent “cancer-like” signal was observed in <1% of NC participants across all assays (representing potential undiagnosed cancers). An increasing trend was observed in NC vs stages I-III vs stage IV (nonsyn. SNVs/indels per Mb [Mean±SD] NC: 1.01±0.86, stages I-III: 2.43±3.98; stage IV: 6.45±6.79; WGS score NC: 0.00±0.08, I-III: 0.27±0.98; IV: 1.95±2.33; methylation score NC: 0±0.50; I-III: 1.02±1.77; IV: 3.94±1.70). These data demonstrate the feasibility of achieving >99% specificity for invasive cancer, and support the promise of cfDNA assay for early cancer detection.

### Example 4—Example Cell Sources

In some embodiments, a cell source of any embodiment of the present disclosure is a first cancer condition of a common primary site of origin. In some embodiments, the first cancer condition is breast cancer, lung cancer, prostate cancer, colorectal cancer, renal cancer, uterine cancer, pancreatic cancer, cancer of the esophagus, a lymphoma, head/neck cancer, ovarian cancer, a hepatobiliary cancer, a melanoma, cervical cancer, multiple myeloma, leukemia, thyroid cancer, bladder cancer, gastric cancer, or a combination thereof.

In some embodiments, a cell source of any embodiment of the present disclosure is a tumor of a certain cancer type, or a fraction thereof. In some embodiments, the tumor is an adrenocortical carcinoma, a childhood adrenocortical carcinoma, a tumor of an AIDS-related cancer, kaposi sarcoma, a tumor associated with anal cancer, a tumor associated with an appendix cancer, an astrocytoma, a childhood (brain cancer) tumor, an atypical teratoid/rhabdoid tumor, a central nervous system (brain cancer) tumor, a basal cell carcinoma of the skin, a tumor associated with bile duct cancer, a bladder cancer tumor, a childhood bladder cancer tumor, a bone cancer (e.g., ewing sarcoma and osteosarcoma and malignant fibrous histiocytoma) tissue, a brain tumor, breast cancer tissue, childhood breast cancer tissue, a childhood bronchial tumor, burkitt lymphoma tissue, a carcinoid tumor (gastrointestinal), a childhood carcinoid tumor, a carcinoma of unknown primary, a childhood carcinoma of unknown primary, a childhood cardiac (heart) tumor, a central nervous system (e.g., brain cancer such as childhood atypical teratoid/rhabdoid) tumor, a childhood embryonal tumor, a childhood germ cell tumor, cervical cancer tissue, childhood cervical cancer tissue, cholangiocarcinoma tissue, childhood chordoma tissue, a chronic myeloproliferative neoplasm, a colorectal cancer tumor, a childhood colorectal cancer tumor, childhood craniopharyngioma tissue, a ductal carcinoma in situ (DCIS), a childhood embryonal tumor, endometrial cancer (uterine cancer) tissue, childhood ependymoma tissue, esophageal cancer tissue, childhood esophageal cancer tissue, esthesioneuroblastoma (head and neck cancer) tissue, a childhood extracranial germ cell tumor, an extragonadal germ cell tumor, eye cancer tissue, an intraocular melanoma, a retinoblastoma, fallopian tube cancer tissue, gallbladder cancer tissue, gastric (stomach) cancer tissue, childhood gastric (stomach) cancer tissue, a gastrointestinal carcinoid tumor, a gastrointestinal stromal tumor (GIST), a childhood gastrointestinal stromal tumor, a germ cell tumor (e.g., a childhood central nervous system germ cell tumor, a childhood extracranial germ cell tumor, an extragonadal germ cell tumor, an ovarian germ cell tumor, or testicular cancer tissue), head and neck cancer tissue, a childhood heart tumor, hepatocellular cancer (HCC) tissue, an islet cell tumor (pancreatic neuroendocrine tumors), kidney or renal cell cancer (RCC) tissue, laryngeal cancer tissue, leukemia, liver cancer tissue, lung cancer (non-small cell and small cell) tissue, childhood lung cancer tissue, male breast cancer tissue, a malignant fibrous histiocytoma of bone and osteosarcoma, a melanoma, a childhood melanoma, an intraocular melanoma, a childhood intraocular melanoma, a merkel cell carcinoma, a malignant mesothelioma, a childhood mesothelioma, metastatic cancer tissue, metastatic squamous neck cancer with occult primary tissue, a midline tract carcinoma with NUT gene changes, mouth cancer (head and neck cancer) tissue, multiple endocrine neoplasia syndrome tissue, a multiple myeloma/plasma cell neoplasm, myelodysplastic syndrome tissue, a myelodysplastic/myeloproliferative neoplasm, a chronic myeloproliferative neoplasm, nasal cavity and paranasal sinus cancer tissue, nasopharyngeal cancer (NPC) tissue, neuroblastoma tissue, non-small cell lung cancer tissue, oral cancer tissue, lip and oral cavity cancer and oropharyngeal cancer tissue, osteosarcoma and malignant fibrous histiocytoma of bone tissue, ovarian cancer tissue, childhood ovarian cancer tissue, pancreatic cancer tissue, childhood pancreatic cancer tissue, papillomatosis (childhood laryngeal) tissue, paraganglioma tissue, childhood paraganglioma tissue, paranasal sinus and nasal cavity cancer tissue, parathyroid cancer tissue, penile cancer tissue, pharyngeal cancer tissue, pheochromocytoma tissue, childhood pheochromocytoma tissue, a pituitary tumor, a plasma cell neoplasm/multiple myeloma, a pleuropulmonary blastoma, a primary central nervous system (CNS) lymphoma, primary peritoneal cancer tissue, prostate cancer tissue, rectal cancer tissue, a retinoblastoma, a childhood rhabdomyosarcoma, salivary gland cancer tissue, a sarcoma (e.g., a childhood vascular tumor, osteosarcoma, uterine sarcoma, etc.), Sézary syndrome (lymphoma) tissue, skin cancer tissue, childhood skin cancer tissue, small cell lung cancer tissue, small intestine cancer tissue, a squamous cell carcinoma of the skin, a squamous neck cancer with occult primary, a cutaneous t-cell lymphoma, testicular cancer tissue, childhood testicular cancer tissue, throat cancer (e.g., nasopharyngeal cancer, oropharyngeal cancer, hypopharyngeal cancer) tissue, a thymoma or thymic carcinoma, thyroid cancer tissue, transitional cell cancer of the renal pelvis and ureter tissue, unknown primary carcinoma tissue, ureter or renal pelvis tissue, transitional cell cancer (kidney (renal cell) cancer tissue, urethral cancer tissue, endometrial uterine cancer tissue, uterine sarcoma tissue, vaginal cancer tissue, childhood vaginal cancer tissue, a vascular tumor, vulvar cancer tissue, a Wilms tumor or other childhood kidney tumor.

In some embodiments, a cell source of any embodiment of the present disclosure is a first cancer condition. In some such embodiments, the first cancer condition is a stage of a breast cancer, a stage of a lung cancer, a stage of a prostate cancer, a stage of a colorectal cancer, a stage of a renal cancer, a stage of a uterine cancer, a stage of a pancreatic cancer, a stage of a cancer of the esophagus, a stage of a lymphoma, a stage of a head/neck cancer, a stage of a ovarian cancer, a stage of a hepatobiliary cancer, a stage of a melanoma, a stage of a cervical cancer, a stage of a multiple myeloma, a stage of a leukemia, a stage of a thyroid cancer, a stage of a bladder cancer, or a stage of a gastric cancer.

In some embodiments, a cell source of any embodiment of the present disclosure is a predetermined stage of a breast cancer, a predetermined stage of a lung cancer, a predetermined stage of a prostate cancer, a predetermined stage of a colorectal cancer, a predetermined stage of a renal cancer, a predetermined stage of a uterine cancer, a predetermined stage of a pancreatic cancer, a predetermined stage of a cancer of the esophagus, a predetermined stage of a lymphoma, a predetermined stage of a head/neck cancer, a predetermined stage of a ovarian cancer, a predetermined stage of a hepatobiliary cancer, a predetermined stage of a melanoma, a predetermined stage of a cervical cancer, a predetermined stage of a multiple myeloma, a predetermined stage of a leukemia, a predetermined stage of a thyroid cancer, a predetermined stage of a bladder cancer, or a predetermined stage of a gastric cancer.

In some embodiments, a cell source of any embodiment of the present disclosure is from a non-cancerous tissue. In some embodiments, a cell source of any embodiment of the present disclosure is from cells that derive from healthy tissue. In some embodiments, a cell source of any embodiment of the present disclosure is from a healthy tissue such as breast, lung, prostate, colorectal, renal, uterine, pancreatic, esophageal, lymph, ovarian, cervical, epidermal, thyroid, bladder, gastric, or a combination thereof.

In some embodiments, a cell source of any embodiment of the present disclosure is derived from one tissue type. In some embodiments, a cell source of any embodiment of the present disclosure is derived from two or more tissue types. In some embodiments, a tissue type includes one or more cell types (e.g., a combination of healthy, non-cancerous cells and cancerous cells). In some embodiments, a tissue type includes one cell type (e.g., one of either cancerous or healthy, non-cancerous cells).

In some embodiments, a cell source of any embodiment of the present disclosure constitutes one cell type, two cell types, three cell types, four cell types, five cell types, six cell types, seven cell types, eight cell types, nine cell types, ten cell types, or more than ten cell types.

In some embodiments, a cell source of any embodiment of the present disclosure is liver cells. In some such embodiments, the cell source is hepatocytes, hepatic stellate fat storing cells (ITO cells), Kupffer cells, sinusoidal endothelial cells, or any combination thereof.

In some embodiments, a cell source of any embodiment of the present disclosure is stomach cells. In some such embodiments, the cell source is parietal cells.

In some embodiments, a cell source of any embodiment of the present disclosure is one or more types of human cells. In some such embodiments, the cell source is adaptive NK cells, adipocytes, alveolar cells, Alzheimer type II astrocytes, amacrine cells, ameloblasts, astrocytes, B cells, basophils, basophil activation cells, basophilia cells, Betz cells, bistratified cells, Boettcher cells, cardiac muscle cells, CD4+ T cells, cementoblasts, cerebellar granule cells, cholangiocytes, cholecystocytes, chromaffin cells, cigar cells, club cells, orticotropic cells, cytotoxic T cells, dendritic cells, enterochromaffin cells, enterochromaffin-like cells, eosinophils, extraglomerular mesangial cells, faggot cells, fat pad cells, gastric chief cells, goblet cells, gonadotropic cells, hepatic stellate cells, hepatocytes, hypersegmented neutrophils, intraglomerular mesangial cells, juxtaglomerular cells, keratinocytes, kidney proximal tubule brush border cells, Kupffer cells, lactotropic cells, Leydig cells, macrophages, macula densa cells, mast cells, megakaryocytes, melanocytes, microfold cells, monocytes, natural killer cells, natural killer T cells, glitter cells, neutrophils, osteoblasts, osteoclasts, osteocytes, oxyphil cells (parathyroid), paneth cells, parafollicular cells, parasol cells, parathyroid chief cells, parietal cells, parvocellular neurosecretory cells, peg cells, pericytes, peritubular myoid cells, platelets, podocytes, regulatory T cell, reticulocytes, retina bipolar cells retina horizontal cells, retinal ganglion cells, retinal precursor cells, sentinel cells, sertoli cells, somatomammotrophic cells, somatotropic cells, stellate cells, sustentacular cells, T cells, T helper cells, telocytes, tendon cells, thyrotropic cells, transitional B cells, trichocytes (human), tuft cells, unipolar brush cells, white blood cells, zellballens, or any combination thereof. In some such embodiments, such cells of the cell source are healthy. In alternative embodiments such cells of the cell source are afflicted with cancer.

In some embodiments, a cell source of any embodiment of the present disclosure is any combination of cell types provided that such cell types originated from a single organ. In some such embodiments this single organ is breast, lung, prostate, colon/rectum, kidney, uterus, pancreas, esophagus, blood, head/neck, ovary, liver, cervix, thyroid, bladder, or stomach. In some embodiments this single organ is healthy. In alternative embodiments this single organ is afflicted with cancer that originated in the single organ. In still further alternative embodiments, this single organ is afflicted with cancer that originated in an organ other than the single organ and metastasized to the single organ.

In some embodiments, a cell source of any embodiment of the present disclosure is any combination of cell types provided that such cell types originated from a predetermined set of organs. In some such embodiments this predetermined set of organs is any two organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some embodiments, a cell source of any embodiment of the present disclosure is any combination of cell types provided that such cell types originated from a predetermined set of organs. In some such embodiments this predetermined set of organs is any three organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some embodiments, a cell source of any embodiment of the present disclosure is any combination of cell types provided that such cell types originated from a predetermined set of organs. In some such embodiments this predetermined set of organs is any four organs, five organs, six organs, or seven organs in the set breast, lung, prostate, colon/rectum, kidney, uterus, pancreas, esophagus, blood, head/neck, ovary, liver, cervix, thyroid, bladder, and stomach. In some embodiments this predetermined set of organs is healthy. In alternative embodiments this predetermined set of organs is afflicted with cancer that originated in one of the organs in the predetermined set of organs. In still further alternative embodiments, the predetermined set of organs is afflicted with cancer that originated in an organ other than the predetermined set of organs and metastasized to the predetermined set of organs.

In some specific embodiments, a cell source of any embodiment of the present disclosure is white blood cells. In some such embodiments, the cell source is neutrophils, eosinophils, basophils, lymphocytes, B lymphocytes, T lymphocytes, cytotoxic T cells, monocytes, or any combination thereof.

### CONCLUSION

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (“or “in response to detecting (the stated condition or event),” depending on the context.

The foregoing description included example systems, methods, techniques, instruction sequences, and computing machine program products that embody illustrative implementations. For purposes of” explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1-90. (canceled)

91. A method of estimating cell source fraction for a subject, the method comprising:
at a computer system having one or more processors, and memory storing one or more programs for execution by the one or more processors:
obtaining, in electronic form, a corresponding methylation pattern of each respective cell-free fragment in a plurality of cell-free fragments, wherein the plurality of cell-free fragments comprises at least 4000 cell-free fragments, and wherein the corresponding methylation pattern of each respective cell-free fragment (i) is determined by a methylation sequencing of one or more nucleic acid samples comprising the respective fragment in a biological sample obtained from the subject and (ii) comprises a methylation state of each CpG site in a corresponding plurality of CpG sites in the respective fragment;
mapping each cell-free fragment in the plurality of cell-free fragments to a bin in a plurality of bins, wherein the plurality of bins comprises 1000 bins, thereby obtaining a plurality of sets of cell-free fragments, each set of cell-free fragments mapped to a different bin in the plurality of bins;
assigning a cell-free fragment cancer condition to each respective cell-free fragment in each set of cell-free fragments in the plurality of sets of cell-free fragments, wherein the cell-free fragment cancer condition is one of the first cancer condition and the second cancer condition, as a function of an output of a classifier upon inputting a methylation pattern of the respective cell-free fragment into the classifier;
computing a first measure of central tendency of the number of cell-free fragments from the subject that have been assigned the first cancer condition in each set of cell-free fragments across the plurality of bins;
computing a second measure of central tendency of the number of cell-free fragments from the subject in each set of cell-free fragments across the plurality of bins; and
estimating the cell source fraction for the subject using the first measure of central tendency and the second measure of central tendency.

92-94. (canceled)

95. The method of claim 91, wherein each respective bin in the plurality of bins has, on average, between 10 and 10000 residues.

96. The method of claim 91, wherein
the first measure of central tendency is an arithmetic mean, a weighted mean, a midrange, a midhinge, a trimean, a Winsorized mean, a mean, or a mode of the number of cell-free fragments from the subject that have been assigned the first cancer condition in each set of cell-free fragments across the plurality of bins, and
the second measure of central tendency is an arithmetic mean, a weighted mean, a midrange, a midhinge, a trimean, a Winsorized mean, a mean, or a mode of the number of cell-free fragments from the subject in each set of cell-free fragments across the plurality of bins.

97. (canceled)

98. The method of claim 91, wherein the estimating the cell source fraction comprises dividing the first measure of central tendency by the second measure of central tendency.

99. The method of claim 91, wherein the methylation sequencing is paired-end sequencing or single-read sequencing.

100. (canceled)

101. The method of claim 91, wherein cell free fragment in the plurality of cell-free fragments has an average length of less than 500 nucleotides.

102. The method of claim 91, wherein the first cancer condition is cancer and the second cancer condition is absence of cancer.

103-104. (canceled)

105. The method of claim 91, wherein the methylation sequencing is whole genome methylation sequencing.

106. The method of claim 91, wherein the methylation sequencing is targeted sequencing using a plurality of nucleic acid probes and each respective bin in the plurality of bins is associated with at least one corresponding nucleic acid probe in the plurality of nucleic acid probes.

107. The method of claim 106, wherein the plurality of nucleic acid probes comprises 1,000 or more nucleic acid probes, 2,000 or more nucleic acid probes, 3,000 or more nucleic acid probes, 5,000 or more nucleic acid probes, 10,000 or more nucleic acid probes or between 1,000 nucleic acid probes and 30,000 nucleic acid probes.

108. The method of claim 91, wherein each bin in the plurality of bins comprises 2, 3, 4, 5, 6, 7, 8, 9, 10, 11, 12, 13, 14, 15, 16, 17, 18, 19, 20 or more CpG sites.

109-112. (canceled)

113. The method of claim 91, wherein the biological sample comprises blood, whole blood, plasma, serum, urine, cerebrospinal fluid, fecal, saliva, sweat, tears, pleural fluid, pericardial fluid, or peritoneal fluid of the subject.

114-115. (canceled)

116. The method of claim 91, wherein the methylation sequencing detects one or more 5-methylcytosine (5mC) and/or 5-hydroxymethylcytosine (5hmC) in the respective fragment.

117-119. (canceled)

120. The method of claim 91, wherein:
the classifier used for assigning a cell-free fragment condition comprises a first model for the first cancer condition and a second model for the second cancer condition, wherein:
the first model is a first mixture model comprising a first plurality of sub-models,
the second model is a second mixture model comprising a second plurality of sub-models, and
each sub-model in the first and second plurality of sub-models represents an independent corresponding methylation model for a source of cell-free fragments in the corresponding biological sample.

121. The method of claim 120, wherein each independent corresponding methylation model is one of a binomial model, beta-binomial model, independent sites model or Markov model.

122. The method of claim 120, wherein:
two or more sub-models in the first plurality of sub-models are independent sites models, and
two or more sub-models in the second plurality of sub-models are independent sites models.

123. The method of claim 91, further comprising, prior to the mapping B), applying one or more filter conditions to the plurality of cell-free fragments, wherein
a filter condition in the one or more filter conditions is application of a p-value threshold to the corresponding methylation pattern for each respective cell-free fragment in the plurality of cell-free fragments, wherein the p-value threshold is representative of how frequently a methylation pattern is observed in a cohort of non-cancer subjects,
a filter condition in the one or more filter conditions is application of a requirement that each respective cell-free fragment in the plurality of cell-free fragments is represented by a threshold number of sequence reads in a corresponding plurality of sequence reads measured from the one or more nucleic acid samples comprising the respective fragment in the corresponding biological sample,
a filter condition in the one or more filter conditions is application of a requirement that each respective cell-free fragment in the plurality of cell-free fragments is represented by a threshold number of cell-free nucleic acids in the one or more nucleic acid samples comprising the respective fragment in the corresponding biological sample,
a filter condition in the one or more filter conditions is application of a requirement that each respective cell-free fragment in the plurality of cell-free fragments have a threshold number of CpG sites, or
a filter condition in the one or more filter conditions is a requirement that each respective cell-free fragment in the plurality of cell-free fragments have a length of less than a threshold number of base pairs.

124-135. (canceled)

136. The method of claim 91, the method further comprising:
applying a treatment regimen to the subject based at least in part, on a value of the cell source fraction for the subject.

137. The method of claim 136, wherein the treatment regimen comprises applying an agent for cancer to the subject, wherein the agent for cancer is a hormone, an immune therapy, radiography, or a cancer drug.

138-139. (canceled)

140. The method claim 91, wherein the subject has been treated with an agent for cancer, wherein the agent for cancer is a hormone, an immune therapy, radiography, or a cancer drug, and the method further comprises:
using the cell source fraction for the subject to evaluate a response of the subject to the agent for cancer.

141-142. (canceled)

143. The method of claim 91, wherein the subject has been treated with an agent for cancer and the method further comprises:
using the cell source fraction for the subject to determine whether to intensify or discontinue the agent for cancer in the subject.

144. The method of claim 91, wherein the subject has been subjected to a surgical intervention to address the cancer and the method further comprises:
using the cell source fraction for the subject to evaluate a condition of the subject in response to the surgical intervention.

145. The method of claim 91, the method further comprising:
repeating the obtaining, mapping, assigning, computing the first and second measure of central tendency, and estimating the cell source fraction for the subject at each respective time point in a plurality of time points across an epoch, thereby obtaining a corresponding cell source fraction, in a plurality of cell source fractions, for the subject at each respective time point; and
using the plurality of cell source fractions to determine a state or progression of a disease condition in the subject during the epoch in the form of an increase or decrease of a first cell source fraction over the epoch.

146. The method of claim 145, wherein the epoch is a period of hours, months, or years and each time point in the plurality of time points is a different time point in the period of hours, months, or years.

147-151. (canceled)

152. The method of claim 145, the method further comprising changing a diagnosis, prognosis, or treatment of the subject when the first cell source fraction of the subject is observed to change by a threshold amount across the epoch.

153-154. (canceled)

155. The method of claim 152, wherein the threshold is greater than ten percent, greater than twenty percent, greater than thirty percent, greater than forty percent, greater than fifty percent, greater than two-fold, greater than three-fold, or greater than five-fold.

156. The method of claim 91, wherein the cell source fraction is a tumor fraction.

157. (canceled)

158. The method of claim 91, wherein a bin in the plurality of bins corresponds to a genomic region listed in one or more of Tables 1-24 of International Publication No. WO2019/195268A2, lists 1-16 of International Publication No. WO2020/154682A2, and/or lists 1-8 of International Publication No. WO2020/069350A1.

159-162. (canceled)

163. The method of claim 91, wherein the plurality of cell-free fragments, for the subject, comprises at least 100,000 cell-free fragments.

164-166. (canceled)

167. A computer system for estimating cell source fraction for a subject, the computer system comprising:
one or more processors; and
a memory, the memory storing one or more programs for execution by the one or more processors, the one or more programs comprising instructions for:
obtaining, in electronic form, a corresponding methylation pattern of each respective cell-free fragment in a plurality of cell-free fragments, wherein the plurality of cell-free fragments comprises at least 4000 cell-free fragments, and wherein the corresponding methylation pattern of each respective cell-free fragment (i) is determined by a methylation sequencing of one or more nucleic acid samples comprising the respective fragment in a biological sample obtained from the subject and (ii) comprises a methylation state of each CpG site in a corresponding plurality of CpG sites in the respective fragment;
mapping each cell-free fragment in the plurality of cell-free fragments to a bin in a plurality of bins, wherein the plurality of bins comprises 1000 bins, thereby obtaining a plurality of sets of cell-free fragments, each set of cell-free fragments mapped to a different bin in the plurality of bins;
assigning a cell-free fragment cancer condition to each respective cell-free fragment in each set of cell-free fragments in the plurality of sets of cell-free fragments, wherein the cell-free fragment cancer condition is one of the first cancer condition and the second cancer condition, as a function of an output of a classifier upon inputting a methylation pattern of the respective cell-free fragment into the classifier;
computing a first measure of central tendency of the number of cell-free fragments from the subject that have been assigned the first cancer condition in each set of cell-free fragments across the plurality of bins;
computing a second measure of central tendency of the number of cell-free fragments from the subject in each set of cell-free fragments across the plurality of bins; and
estimating the cell source fraction for the subject using the first measure of central tendency and the second measure of central tendency.

168. A non-transitory computer-readable storage medium having stored thereon program code instructions that, when executed by a processor, cause the processor to perform a method of estimating cell source fraction for a subject, the method comprising:
obtaining, in electronic form, a corresponding methylation pattern of each respective cell-free fragment in a plurality of cell-free fragments, wherein the plurality of cell-free fragments comprises at least 4000 cell-free fragments, and wherein the corresponding methylation pattern of each respective cell-free fragment (i) is determined by a methylation sequencing of one or more nucleic acid samples comprising the respective fragment in a biological sample obtained from the subject and (ii) comprises a methylation state of each CpG site in a corresponding plurality of CpG sites in the respective fragment;
mapping each cell-free fragment in the plurality of cell-free fragments to a bin in a plurality of bins, wherein the plurality of bins comprises 1000 bins, thereby obtaining a plurality of sets of cell-free fragments, each set of cell-free fragments mapped to a different bin in the plurality of bins;
assigning a cell-free fragment cancer condition to each respective cell-free fragment in each set of cell-free fragments in the plurality of sets of cell-free fragments, wherein the cell-free fragment cancer condition is one of the first cancer condition and the second cancer condition, as a function of an output of a classifier upon inputting a methylation pattern of the respective cell-free fragment into the classifier;
computing a first measure of central tendency of the number of cell-free fragments from the subject that have been assigned the first cancer condition in each set of cell-free fragments across the plurality of bins;
computing a second measure of central tendency of the number of cell-free fragments from the subject in each set of cell-free fragments across the plurality of bins; and
estimating the cell source fraction for the subject using the first measure of central tendency and the second measure of central tendency.
